(19) United States
(12) Patent Application Publication
(10) Pub. No.: US 2024/0409895 A1
(43) Pub. Date: Dec. 12, 2024

(54) IL-4 EXPOSED MACROPHAGE EXOSOMES AND METHODS OF USE THEREOF

(71) Applicants: The United States Government As Represented By The Department Of Veterans Affairs, Washington, DC (US); The Regents Of The University Of California, Oakland, CA (US)

(72) Inventors: Robert L. Raffai, San Francisco, CA (US); Tuan Anh Phu, San Francisco, CA (US); Ngan Khanh Vu, San Francisco, CA (US); Martin Ng, San Francisco, CA (US); Laura Bouchareychas, San Francisco, CA (US)

(21) Appl. No.: 18/424,086

(22) Filed: Jan. 26, 2024

Related U.S. Application Data
(60) Provisional application No. 63/481,729, filed on Jan. 26, 2023.

(51) Int. Cl. C12N 5/0786

(52) U.S. Cl. CPC C12N 5/0645; C12N 2501/2304; C12N 2501/65

(57) ABSTRACT

Disclosed are method of producing IL-4 exposed M2 macrophage exosomes comprising culturing macrophage or macrophage precursor cells with IL-4 in culture media, and isolating exosomes from the culture media, wherein the isolated exosomes are IL-4 exposed M2 macrophage exosomes enriched with miR-21, miR-99a, miR-146b and miR378a. Disclosed are methods of reprogramming macrophages and/or adipocytes comprising exposing the macrophages and/or adipocytes to IL-4 exposed M2 macrophage exosomes, wherein immune and/or metabolic properties are altered in the macrophages and/or adipocytes. Methods of treating with IL-4 exposed M2 macrophage exosomes.

### CROSS-REFERENCE TO RELATED APPLICATIONS

This application claims the benefit of U.S. Provisional Patent Application No. 63/481,729, filed Jan. 26, 2023, which is incorporated by reference herein in its entirety.

### STATEMENT REGARDING FEDERALLY SPONSORED RESEARCH

This invention was made with government support under Grant Numbers R01HL133575 and 1UG3CA241703 awarded by the National Institutes of Health. The government has certain rights in this invention.

### BACKGROUND

Cardiometabolic inflammatory disease and its associated complications are leading causes of morbidity and mortality due to the increasing prevalence of diabetes. Risk factors contributing to its pathogenesis include obesity, insulin-resistance, dyslipidemia, and hypertension. Recent findings point to chronic, unresolved inflammation as a major contributor to the onset and progression of cardiometabolic disease and its complications. A hallmark of this inflammatory response includes an accumulation of pro-inflammatory M1-like macrophages in the liver, as well as in adipose and vascular tissues. A number of studies have identified the release of pro-inflammatory cytokines including tumor necrosis factor-alpha (TNF-α) and interleukin-1-beta (IL-1β) from tissue-associated M1-like macrophages as factors that further exacerbates local inflammation and inhibit insulin sensitivity. In contrast, anti-inflammatory M2-like macrophages have been shown to exert protective properties in the liver, as well as in adipose and vascular tissues. While polarizing adipose tissue macrophages to an M2-like phenotype protects high fat diet-fed mice against obesity-induced insulin resistance and induces beiging in white adipose tissues, key signaling factors that could be exploited to drive this beneficial phenomenon remain elusive.

Extracellular vesicles (EVs) including exosomes have recently been recognized as sources of intercellular communication in numerous disease states including inflammation and metabolic disease. EVs, including exosomes are increasing reported to serve in modulating obesity beyond cytokines and other signaling factors. Specifically, exosomes produced by macrophages have been shown to control numerous disease states through the delivery of cargo including microRNA.

### BRIEF SUMMARY

Disclosed are method of producing IL-4 exposed M2 macrophage exosomes comprising culturing macrophage or macrophage precursor cells in the presence of IL-4 in the culture media to thereby produce IL-4 exposed M2 macrophage exosomes, and isolating the IL-4 exposed M2 macrophage exosomes from the culture media, wherein the IL-4 exposed M2 macrophage exosomes are enriched with miR-21, miR-99a, miR-146b and miR378a. In some aspects, the macrophage or macrophage precursor cells are exposed to PMA prior to culturing the macrophage or macrophage precursor cells in the presence of IL-4.

Disclosed are methods of reprogramming macrophages comprising exposing the macrophages to IL-4 exposed M2 macrophage exosomes, wherein immune and/or metabolic properties are altered in the macrophages.

Disclosed are methods of reprogramming adipocytes comprising exposing the adipocytes to IL-4 exposed M2 macrophage exosomes, wherein immune and/or metabolic properties are altered in the adipocytes.

Disclosed are methods of treating diseases or disorders in a subject in need thereof comprising administering to the subject in need thereof a therapeutically effective amount of the M2 macrophage exosomes disclosed herein. The disease or disorder treated can be diabetes, cardiometabolic disease, chronic inflammatory disorder, atherosclerosis, cardiac inflammation after myocardial infarction, septicemia, lethargy, or pulmonary inflammation. Thus, in some aspects, disclosed are methods of treating type II diabetes, cardiometabolic disease, chronic and acute inflammatory disorder, sepsis, pulmonary infection or lethargy in a subject in need thereof comprising administering to the subject in need thereof a therapeutically effective amount of the M2 macrophage exosomes disclosed herein.

Disclosed are methods of reducing acute pulmonary and cardiac leukocyte infiltration, in a subject in need thereof comprising administering to the subject in need thereof a therapeutically effective amount of the M2 macrophage exosomes disclosed herein.

Disclosed are methods of enhancing oxidative phosphorylation (OXPHOS) in a cell of a subject in need thereof comprising administering to the subject in need thereof a therapeutically effective amount of the M2 macrophage exosomes disclosed herein.

Disclosed are methods of inducing white adipose tissue beiging in a subject in need thereof comprising administering to the subject in need thereof a therapeutically effective amount of the M2 macrophage exosomes disclosed herein.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1E show biophysical parameters of THP-1 macrophage exosomes. (A) Representative concentration and size distributions of THP1-WT-exo & THP1-IL-4-exo purified from THP-1 cell culture supernatants after a 24 h period of culture as determined using nanoparticle tracking analysis. (B and C) Average concentration of purified exosomes in particles/mL (B) and mode of particle diameter in nm (C) (n=4 samples per group). (D) Electron micrograph of purified exosomes from THP-1 cells. Scale bar, 100 nm. (E) Western blot analysis of Calnexin, GM130, CD9, CD63, and CD81 in exosome-free media (EFM), cell lysate, and 1.5×10⁹ particles of THP-1-derived exosomes (representative of three independent experiments). Data are represented as mean±SEM.

FIGS. 2A-2G show THP1-IL4-exo modulate inflammation and energy expenditure in recipient macrophages by inducing mitochondrial respiration. (A) Merged images showing and quantification of the internalization of PKH26-labeled THP-1-derived exosomes by naive primary BMDM counterstained with Hoechst (blue). BMDM were co-incubated with 2×10⁹ PKH26-labeled exosomes for 2 h at 37° C. and washed repeatedly to remove unbound exosomes. All images were acquired using a Zeiss Axio microscope system with a 20× objective (n=8 samples per group, pooled from two independent experiments). (B) qRT-PCR analysis of Il1b, Tnf, Mcp1, Arg1, Chil3, and Retnla mRNA expression in BMDMs treated with PBS (control), THP1-WT-exo or THP1-IL-4-exo for 24 h. Results were normalized to B2m and Gapdh mRNA and are presented relative to control (n=12 per group, pooled from three independent experiments). (C) Graph showing representative Seahorse mitochondrial stress tests. O, oligomycin (1 μM); F, FCCP (2 μM); R/AA, rotenone/antimycin A (0.5 μM). One representative experiment out of two experiments is shown; n=5 per group. (D) Graphs showing quantified cell-normalized mitochondrial OCR from stress tests; n=10 per group, pooled from two independent experiments. (E-G) Graphs showing MFI of MitoSOX (E), TMRM (F), and Calccin AM (G) signals in BMDM treated with 4×10⁹ particles/mL of THP1-WT-exo, THP1-IL4-exo, or PBS (control) as measured by flow cytometric analysis; n=8 in each group, pooled from two independent experiments. *p<0.05, **p<0.01, and ***p<0.001 as determined using one-way ANOVA and Holm-Sidak post-test. Data are represented as mean±SEM.

FIGS. 3A-3F show THP1-IL4-exo regulate lipid homeostasis, induce lipophagy, and control the expression of distinct microRNAs in recipient macrophages. (A) Merged images showing LipidTOX staining of neutral lipids in naive cultured BMDM counterstained with Hoechst (blue). BMDM were co-incubated with 4×10⁹ particles/mL of THP1-WT-exo, THP1-IL4-exo, or PBS for 24 h at 37° C. and subsequently stained with LipidTOX for 30 min. All images were acquired using a Zeiss microscope system with a 20× objective. Scale bars: 50 μm. (B) Representative flow cytometric plot and graph showing average mean fluorescent intensity (MFI) of LipidTOX signals in BMDM treated with 4×10⁹ particles/mL of THP1-WT-exo, THP1-IL4-exo, or PBS for 24 h at 37° C.; n=12 per group, pooled from three independent experiments. (C) qRT-PCR analysis of Ulk1, Atg5, Atg7, Pnpla2, Lipe, Map1lc3a, and Map1lc3b mRNA expression in BMDMs treated with PBS (control), THP1-WT-exo, or THP1-IL-4-exo for 24 h. (D) qRT-PCR analysis of Pparg, Abca1, Abcg1, Apoe, Srebf1, and Srebf2 expression in BMDMs treated with PBS (control), THP1-WT-exo, or THP1-IL-4-exo for 24 h. Results were normalized to B2m and Gapdh mRNA and are presented relative to control (n=12 per group, pooled from three independent experiments). (E) qRT-PCR analysis of miR-99a-5p, miR-146b-5p, and miR-378a-3p microRNA expression in THP1-WT-exo and THP1-IL-4-exo. Results were normalized to U6 snRNA and miR-16-5p expression, with UniSp6 used as a spike-in control. Data are presented relative to THP1-WT-exo (n=4 per group). (F) qRT-PCR analysis of miR-99a-5p, miR-146b-5p, miR-378a-3p, miR-21-5p, and miR-33-5p microRNA expression in BMDMs treated with PBS (control), THP1-WT-exo, or THP1-IL-4-exo for 24 h. Results were normalized to U6 snRNA and miR-16-5p expression, with UniSp6 used as a spike-in control. Data are presented relative to control (n=12 per group, pooled from three independent experiments). *p<0.05, **p<0.01, and ***p<0.001 as determined using either unpaired Student's t-test (for two-group comparison) or one-way ANOVA followed by Holm-Sidak post-test (for multiple-group comparison). Data are represented as mean±SEM.

FIGS. 4A-4G show THP1-IL4-exo induce energy expenditure and control expression of distinct microRNAs in recipient 3T3-L1 adipocytes. (A) Merged images showing and quantification of the internalization of PKH26-labeled THP-1-derived exosomes by 3T3-L1 adipocytes counterstained with Hoechst (blue). 3T3-L1 adipocytes were co-incubated with 2×10⁹ PKH26-labeled exosomes for 2 h at 37° C. and washed repeatedly to remove unbound exosomes. All images were acquired using a Zeiss Axio microscope system with a 20× objective (n=8 samples per group, pooled from two independent experiments). (B) qRT-PCR analysis of Pparg, Slc2a4, Srebf1, Srebf2, and Ucp1 mRNA expression in 3T3-L1 adipocytes treated with PBS (control), THP1-WT-exo, or THP1-IL-4-exo for 24 h. Results were normalized to B2m and Gapdh mRNA and are presented relative to control (n=12 per group, pooled from three independent experiments). (C-D) Western blot analysis (C) and quantification (D) of GLUT4, PPARγ, and UCP1 protein levels in cell lysates of 3T3-L1 adipocytes treated with PBS (control), THP1-WT-exo, or THP1-IL-4-exo for 24 h. Quantification was performed using ImageJ and data normalized to loading controls β-Actin or GAPDH (n=6 per group, pooled from two independent experiments). (E) Graph showing representative Seahorse mitochondrial stress tests. O, oligomycin (1 μM); F, FCCP (0.25 μM); R/AA, rotenone/antimycin A (0.5 μM). One representative experiment out of two experiments is shown; n=6 per group. (F) Graphs showing quantified cell-normalized mitochondrial OCR from stress tests; n=12 in each group, pooled from two independent experiments. (F) qRT-PCR analysis of miR-99a-5p, miR-146b-5p, miR-378a-3p, miR-21-5p, and miR-33-5p expression levels in 3T3-L1 adipocytes treated with PBS (control), THP1-WT-exo, or THP1-IL-4-exo for 24 h. Results were normalized to U6 snRNA and miR-16-5p expression, with UniSp6 used as a spike-in control. Data are presented relative to control (n=12 per group, pooled from three independent experiments). *p<0.05, **p<0.01, and ***p<0.001 as determined using one-way ANOVA and Holm-Sidak post-test. Data are represented as mean±SEM.

FIGS. 5A-5H show THP1-IL4-exo enhance mitochondrial activity, induce lipophagy, and promote beiging during 3T3-L1 adipocyte differentiation. (A) Merged images showing LipidTOX staining of 3T3-L1 adipocytes counterstained with Hoechst (blue). 3T3-L1 adipocytes were treated with 4×10⁹ particles/mL of THP1-WT-exo, THP1-IL4-exo, or PBS every two days following a 2-day induction period by IBMX, DEX, and bovine insulin. By day 15, cells were stained with LipidTOX for 30 min. All images were acquired using a Zeiss microscope system with a 20× objective. Scale bars: 50 μm. (B) Representative flow cytometric plot and graph showing average mean fluorescent intensity (MFI) of LipidTOX signals in 3T3-L1 adipocytes treated with 4×10⁹ particles/mL of THP1-WT-exo, THP1-IL4-exo, or PBS every two days following the induction period; n=12 per group, pooled from three independent experiments. (C-E) Graphs showing MFI of MitoSOX (C), TMRM (D), and Calcein AM (E) signals in 3T3-L1 adipocytes treated with 4×10⁹ particles/mL of THP1-WT-exo, THP1-IL4-exo, or PBS every two days following the induction period; n=8 per group, pooled from two independent experiments. (F) qRT-PCR analysis of Ucp1, Ppargc1a, Tbx1, Dio2, Zfp516, Prdm16, and Slc25a25 mRNA expression in 3T3-L1 adipocytes treated with 4×10⁹ particles/mL of THP1-WT-exo, THP1-IL4-exo, or PBS every two days following the induction period. (G) qRT-PCR analysis of Ulk1, Atg5, Atg7, Pnpla2, Lipe, Map1lc3a, and Map1lc3b mRNA expression in 3T3-L1 adipocytes treated with 4×10⁹ particles/mL of THP1-WT-exo, THP1-IL4-exo, or PBS every two days following the induction period. (H) qRT-PCR analysis of Adipoq and Lep in 3T3-L1 adipocytes treated with 4×10⁹ particles/mL of THP1-WT-exo, THP1-IL4-exo, or PBS every two days following the induction period. Results were normalized to B2m and Gapdh mRNA and are presented relative to control (n=12 per group, pooled from three independent experiments). *p<0.05, **p<0.01, and ***p<0.001 as determined using one-way ANOVA and Holm-Sidak post-test. Data are represented as mean±SEM.

FIGS. 6A-6K show THP1-IL4-exo resolve systemic inflammation and hematopoiesis in mice with diet-induced obesity and hyperlipidemia. (A-B) Images of DiR fluorescence in blood (A) and organs (B) 6 h post-injection from 20-week-old Western diet-fed Apoe^{h/h} Ldlr^{-/-} mice injected i.p. with PBS as control or 1×10¹⁰ particles of THP1-WT-exo or THP1-IL4-exo. (C) Schematic diagram detailing the duration of Western diet feeding and injection strategy in 26-week-old Apoe^{h/h} Ldlr^{-/-} mice. (D) Quantification of circulating CD11b⁺ cells, neutrophils, Ly6C^{hi} monocytes, and Ly6C^{lo} monocytes in mice injected with PBS or THP1-IL4-exo by flow cytometry; n=10 per group, pooled from three independent experiments. (E) Quantification of splenic CD11b⁺ cells, neutrophils, Ly6C^{hi} monocytes, and Ly6C^{lo} monocytes in mice injected with PBS or THP1-IL4-exo by flow cytometry; n=10 per group, pooled from three independent experiments. (F) Quantification of bone marrow LSK, MPP1, MPP2, MPP3, MPP4, CMP, GMP, and MEP cell populations by flow cytometry; n=8 per group, pooled from two independent experiments. (G) Quantification of splenic LSK, MPP1, MPP2, MPP3, MPP4, CMP, GMP, and MEP cell populations by flow cytometry; n=8 per group, pooled from two independent experiments. (H) qRT-PCR analysis of Tnf, Il1b, and Mcp1 mRNA expression in circulating Ly6C^{hi} monocytes. (I) qRT-PCR analysis of Pparg, Abca1, Abcg1, Srebf1, and Srebf2 mRNA expression in circulating Ly6C^{hi} monocytes. (J) qRT-PCR analysis of miR-99a-5p, miR-146b-5p, miR-378a-3p, miR-21-5p, and miR-33-5p microRNA expression levels in circulating Ly6C^{hi} monocytes isolated by FACS. Results were normalized to B2m and Gapdh for mRNA analysis and U6 snRNA, miR-16-5p, and UniSp6 (spike-in control) for miRNA analysis. Data are presented relative to control (n=8 per group, pooled from two independent experiments). (K) Multiplex immunoassay analysis of plasma TNF-α, IFN-γ, IL-6, and IL-1B; n=10 per group, pooled from three independent experiments. Data are taken in 26-week-old Apoe^{h/h} Ldlr^{-/-} mice fed with a Western diet and injected with PBS or THP1-IL-4-exo (1×10¹⁰ particles/mouse every 2 days for 6 weeks while on Western diet). *p<0.05, **p<0.01, and ***p<0.001 as determined using either unpaired Student's t-test (for two-group comparison) or one-way ANOVA followed by Holm-Sidak post-test (for multiple-group comparison). Data are represented as mean±SEM.

FIGS. 7A-7H show infusions of THP1-IL4-exo suppress aortic, hepatic, and adipose tissue inflammation in obese hyperlipidemic mice. (A and B) Representative flow cytometry plots of leukocyte subsets from aorta (A) and quantification of aortic CD45⁺ cells, macrophages, CD11b⁺ cells, neutrophils, Ly6C^{hi} monocytes, and Ly6C^{lo} monocytes (B). (C and D) Representative flow cytometry plots of leukocyte subsets from livers (C) and quantification of hepatic CD45⁺ cells, infiltrating macrophages/monocytes, Kupffer cells, neutrophils, and Ly6C^{hi} monocytes (D). (E) qRT-PCR analysis of Adgre1, Tnf, Il1b, Mcp1, Nos2, Arg1, Chil3, and Retnla mRNA expression in livers. Results were normalized to B2m and Gapdh mRNA and are presented relative to control. Data are pooled from three independent experiments, n=10 per group. (F) Representative images and quantification of F4/80⁺ cells that formed crown-like structures (CLSs) in WAT; n=8 per group, pooled from two independent experiments. (G) Quantification of eWAT CD45⁺ cells, macrophages, CD11b⁺ cells, neutrophils, and Ly6C^{hi} monocytes measured by flow cytometry. (H) qRT-PCR analysis of Adgre1, Tnf, Il1b, Mcp1, Nos2, Arg1, Chil3, and Retnla mRNA expression in eWAT. Results were normalized to B2m and Gapdh mRNA and are presented relative to control. Data are pooled from three independent experiments, n=10 per group. All data are taken from 26-week-old Apoe^{h/h} Ldlr^{-/-} mice fed with a Western diet and injected with PBS or THP1-IL-4-exo (1×10¹⁰ particles/mouse every 2 days for 6 weeks while on Western diet). *p<0.05, **p<0.01, and ***p<0.001 as determined using either unpaired Student's t-test (for two-group comparison) or one-way ANOVA followed by Holm-Sidak post-test (for multiple-group comparison). Data are represented as mean±SEM.

FIGS. 8A-8N show infusions of THP1-IL4-exo enhance energy expenditure and induce eWAT beiging that improve glucose disposal and normalize fasting blood glucose and insulin levels in hyperlipidemic & obese mice. (A) Fasting glucose and insulin levels measured from mouse plasma; insulin was measured by ELISA. (B and C) Plot showing blood glucose levels in mice during GTT (B) and values of area under the curve in GTT (C). Data are pooled from two independent experiments, n=8 per group. (D) Detection of 2-DG uptake in differentiated 3T3-L1 adipocytes treated with 4×10⁹ particles/mL of THP1-IL4-exo, THP1-WT-exo, or PBS for 24 h; n=10 per group, pooled from two independent experiments. (E) Graph showing representative Seahorse mitochondrial stress tests. O, oligomycin (10 μM); F, FCCP (10 μM); R/AA, rotenone/antimycin A (5 μM). One representative experiment out of two experiments is shown; n=4 per group. (F) Graphs showing quantified protein-normalized mitochondrial OCR from stress tests; n=8 in each group, pooled from two independent experiments. (G) Representative images of hematoxylin & eosin staining and quantification of adipocyte sizes in eWAT; n=8 per group, pooled from two independent experiments. (H) qRT-PCR analysis of Pparg and Slc2a4 mRNA expression in eWAT; n=10 per group, pooled from three independent experiments. (I) Western blot analysis and quantification of GLUT4 protein levels in eWAT tissue lysates. Quantification was performed using ImageJ and data normalized to loading controls Vinculin (n=8 per group, pooled from two independent experiments). (J) qRT-PCR analysis of Ucp1, Ppargc1a, Tbx1, Dio2, Zfp516, Prdm16, and Slc25a25 mRNA expression in eWAT; n=10 per group, pooled from three independent experiments. (K) Images of UCP1 staining in eWAT; two representative images are shown from n=8 per group. (L) qRT-PCR analysis of Ulk1, Atg5, Atg7, Pnpla2, Lipe, Map1lc3a, and Map1lc3b mRNA expression in eWAT; n=10 per group, pooled from three independent experiments. (M) qRT-PCR analysis of Adipoq and Lep mRNA expression in eWAT; n=10 per group, pooled from three independent experiments. (N) Adiponectin: leptin ratio as measured by ELISA from plasma of fasted mice; n=8 per group, pooled from three independent experiments. Data are taken from 26-week-old Apoe^{h/h} Ldlr^{-/-} mice fed with a Western diet and injected with PBS or THP1-IL-4-exo (1×10¹⁰ particles/mouse every 2 days for 6 weeks while on Western diet), or 26-week-old chow-fed wildtype C57BL/6 mice. Results were normalized to B2m and Gapdh mRNA and are presented relative to control. *p<0.05, **p<0.01, and ***p<0.001 as determined using either unpaired Student's t-test (for two-group comparison) or one-way ANOVA followed by Holm-Sidak post-test (for multiple-group comparison). Data are represented as mean±SEM.

FIG. 9 shows THP1-IL4-exo infusions suppressed cardiac inflammation as seen by reduced numbers of leukocyte subsets in HypoE/SR-BI−/− mice following high-fat-diet-induced myocardial infarction. *p<0.05.

FIG. 10 shows THP1-IL4-exo infusions suppressed lung inflammation as seen by reduced numbers of leukocyte subsets in bronchioalveolar lavage of C57BL/6 mice injected with lipopolysaccharide (LPS) and treated with THP1-IL4-exo or PBS. *p<0.05.

FIG. 11 shows THP1-IL4-exo infusions suppressed inflammatory cytokines TNF-alpha, IL-1beta, and IL-6, while increasing levels of the anti-inflammatory cytokine IL-10 in the blood of C57BL/6 mice injected with lipopolysaccharide (LPS) and treated with THP1-IL4-exo or PBS. *p<0.05.

FIG. 12 shows THP1-IL4-exo infusions reduced levels of pro-inflammatory cytokines in plasma of high-fat diet-fed mice with obesity. *p<0.05.

FIGS. 13A-13D show an example of the production and characterization of THP-1 macrophage exosomes. (A) Flow diagram depicting the culture and differentiation of THP-1 macrophages as well as the isolation of their exosomes from conditioned media. (B) Exosomes secretion rate (×10⁹ particles) per million cells over a 24-hour incubation period as detected by NTA. (C) Protein measurements in exosome-containing fractions isolated from conditioned media of THP-1 macrophages treated with or without IL-4. n=4 per group. (D) Western blot analysis of CD63 and CD81 in exosome-free media (EFM), cell lysate, and 1.5 ug proteins of THP-1-derived exosomes (representative of three independent experiments). Data are represented as mean±SEM.

FIGS. 14A-14E show example THP1-IL4-exo modulate inflammatory responses in recipient naïve THP-1 macrophages. (A) qRT-PCR analysis of Tnf, Il1b, Cd86, Cd206, and Cd163 mRNA expression in THP-1 macrophages treated with 2×10⁹ or 4×10⁹ particles/mL THP1-WT-exo, THP1-IL-4-exo, or PBS (control) for 24 h. (B) qRT-PCR analysis of Tnf, Il1b, Cd86, Cd80, Cd206, and Cd163 mRNA expression in THP-1 macrophages treated with 4×10⁹ particles/mL THP1-WT-exo, THP1-IL-4-exo, 20 ng/ml IL-4, or PBS (control) for 24 h. (C) qRT-PCR analysis of Tnf, Il1b, Cd86, Cd80, Cd206, and Cd163 mRNA expression in THP-1 macrophages treated with 4×10⁹ particles/mL THP1-WT-exo, THP1-LPS+IFNg-exo, THP1-IL-4-exo, or PBS (control) for 24 h. Results were normalized to B2m and Actb mRNA and are presented relative to control (representative of three independent measurements, n=4 per group). (D) qRT-PCR analysis of miR-99a, miR-146b-5p, miR-378a-3p, miR-21-5p, and miR-33-5p microRNA expression in THP-1 macrophages treated with 4×10⁹ particles/mL THP1-WT-exo, THP1-IL-4-exo, or PBS (control) for 24 h. Results were normalized to U6 snRNA and miR-16-5p expression, with UniSp6 used as a spike-in control. Data are presented relative to control (representative of three independent measurements, n=4 per group). (E) qRT-PCR analysis of miR-21-5p and miR-33-5p microRNA expression in THP1-WT-exo or THP1-IL-4-exo. Results were normalized to U6 snRNA and miR-16-5p expression, with UniSp6 used as a spike-in control. Data are presented relative to THP1-WT-exo (n=4 per group). *p<0.05, **p<0.01, and ***p<0.001 as determined using either unpaired Student's t-test (for two-group comparison) or one-way ANOVA followed by Holm-Sidak post-test (for multiple-group comparison). Data are represented as mean±SEM.

FIGS. 15A-15C show an example of THP1-IL4-exo induce neutral lipid loss in recipient 3T3-L1 adipocytes & control the expression of miR-378a targets involved in adipocyte beiging. (A) Images showing Oil Red O staining in 3T3-L1 adipocytes treated with 4×10⁹ particles/mL THP1-WT-exo, THP1-IL-4-exo, or PBS (control) every two days following the induction period. All images were acquired using a Zeiss microscope system with a 20× objectives. Scale bars: 50 μm. (B) Quantification of Oil Red O extracted from 3T3-L1 adipocytes by OD 492 nm reading; n=8 per group, pooled from two independent experiments. (C) qRT-PCR analysis of Scd1 and Pde1b mRNA expression in 3T3-L1 adipocytes treated with 4×10⁹ particles/mL THP1-WT-exo, THP1-IL-4-exo, or PBS (control) every two days following the induction period. Results were normalized to B2m and Gapdh mRNA and are presented relative to control (n=12 per group, pooled from three independent experiments). *p<0.05, **p<0.01, and ***p<0.001 as determined using one-way ANOVA followed by Holm-Sidak post-test. Data are represented as mean±SEM.

FIGS. 16A-16I show an example of how THP1-IL4-exo resolve inflammation and control hematopoiesis in hyperlipidemic and obese mice. (A and B) Fasting plasma cholesterol (A) and triglycerides (B). (C and D) Total body weights (C) and percentage of eWAT to total body weight (D). Data are pooled from three independent experiments, n=10 per group. (E) Representative flow cytometry plots of circulating leukocyte subsets. (F) Representative flow cytometry plots of splenic leukocyte subsets. (G and H) Representative flow cytometry plots of bone marrow (G) and splenic (H) hematopoietic stem/progenitor cell subsets. (I) qRT-PCR analysis of Tnf, Il1b, Mcp1, Nos2, Arg1, Chil3, and Retnla mRNA expression in peritoneal macrophages; n=10 per group, pooled from three independent experiments. Data are taken from 26-week-old Apoe^{h/h} Ldlr^{-/-} mice fed with a Western diet and injected with PBS or THP1-IL-4-exo (1×10¹⁰ particles/mouse every 2 days for 6 weeks while on Western diet). Results were normalized to B2m and Gapdh mRNA and are presented relative to control. *p<0.05, **p<0.01, and ***p<0.001 as determined using unpaired Student's t-test. Data are represented as mean±SEM.

FIGS. 17A-17B show THP1-IL4-exo reduce hepatic steatosis in obese Western diet-fed mice. (A) Representative images of hematoxylin & eosin staining and quantification of steatotic areas in livers; n=8 per group, pooled from two independent experiments. (B) Representative images of Oil Red O (ORO) staining and quantification of ORO positive areas in livers; n=8 per group, pooled from two independent experiments. Data are taken from 26-week-old Apoe^{h/h} Ldlr^{-/-} mice fed with a Western diet and injected with PBS or THP1-IL-4-exo (1×10¹⁰ particles/mouse every 2 days for 6 weeks while on Western diet), or 26-week-old chow-fed wildtype C57BL/6 mice. *p<0.05, **p<0.01, and ***p<0.001 as determined using either unpaired Student's t-test (for two-group comparison) or one-way ANOVA followed by Holm-Sidak post-test (for multiple-group comparison). Data are represented as mean±SEM.

FIG. 18 shows an example flow cytometry analysis of leukocyte subsets from eWAT of obese and hyperlipidemic Apoe^{h/h} Ldlr-mice. Representative flow cytometry plots of leukocyte subsets from eWAT of 26-week-old Apoe^{h/h} Ldlr-mice fed with a Western diet and injected with PBS or THP1-IL-4-exo (1×10¹⁰ particles/mouse every 2 days for 6 weeks while on Western diet).

FIGS. 19A-19H show an example that THP1-IL4-exo resolve inflammation in the liver and adipose tissue of obese Western diet-fed wildtype mice. (A and B) Fasting plasma cholesterol (A) and triglycerides (B). (C and D) Total body weights (C) and percentage of eWAT to total body weight (D). Data are pooled from two independent experiments, n=10 per group. (E and F) Representative flow cytometry plots of leukocyte subsets from livers (E) and quantification of hepatic CD45⁺ cells, infiltrating macrophages/monocytes, Kupffer cells, neutrophils, and Ly6C^{hi} monocytes (F). (G and H) Representative flow cytometry plots of leukocyte subsets from eWAT (G) and quantification of adipose CD45⁺ cells, macrophages, CD11b⁺ cells, neutrophils, and Ly6C^{hi} monocytes (H). Data are pooled from two independent experiments, n=10 per group. All data are taken from 26-week-old wildtype C57BL/6 mice fed with a Western diet and injected with PBS or THP1-IL-4-exo (1×10¹⁰ particles/mouse every 2 days for 6 weeks while on Western diet). *p<0.05, **p<0.01, and ***p<0.001 as determined using unpaired Student's t-test. Data are represented as mean±SEM.

FIGS. 20A-20C show an example that THP1-IL4-exo enhance glucose disposal and insulin sensitivity in obese Western diet-fed wildtype mice. (A) Fasting glucose and insulin levels measured in mouse plasma; insulin levels were measured using ELISA. (B and C) Plot showing blood glucose levels in mice during GTT (B) and values of area under the curve in GTT (C). Data are taken from 26-week-old wildtype C57BL/6 mice fed with a Western diet and injected with PBS or THP1-IL-4-exo (1×10¹⁰ particles/mouse every 2 days for 6 weeks while on Western diet); n=10 per group, pooled from two independent experiments. *p<0.05, **p<0.01, and ***p<0.001 as determined using unpaired Student's t-test. Data are represented as mean±SEM.

FIGS. 21A-21B show an example that THP1-IL4-exo reduce expression of genes regulating white adipose tissue beiging in obese Western diet-fed mice. (A and B) qRT-PCR analysis of Scd1 and Pde1b (A) and Nadk (B) mRNA expression in eWAT. Data are taken from 26-week-old Apoc^{h/h} Ldlr^{-/-} mice fed with a Western diet and injected with PBS or THP1-IL-4-exo (1×10¹⁰ particles/mouse every 2 days for 6 weeks while on Western diet). Results were normalized to B2m and Gapdh mRNA and are presented relative to control (n=10 per group, pooled from three independent experiments). *p<0.05, **p<0.01, and ***p<0.001 as determined using unpaired Student's t-test. Data are represented as mean±SEM.

FIG. 22 shows a table of antibodies used for immunohistology, immunoblotting, and flow cytometry applications.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a exosome” includes a plurality of such exosomes, reference to “the macrophage” is a reference to one or more macrophages and equivalents thereof known to those skilled in the art, and so forth.

By “treat” is meant to administer an IL-4 exposed M2 macrophage exosome or composition of the invention to a subject, such as a human or other mammal (for example, an animal model), that has an increased susceptibility for developing a disease, disorder or infection (e.g. diabetes, cardiometabolic disease, chronic inflammatory disorder, cardiac inflammation, septicemia, lethargy, or pulmonary infection) in order to prevent or delay onset of the disease disorder or infection, prevent or delay a worsening of the effects of the disease, disorder or infection, or to partially or fully reverse the effects of the disease, disorder or infection. In some aspects, treat can mean to ameliorate a symptom of a disease, disorder or infection.

By “prevent” is meant to minimize the chance that a subject who has an increased susceptibility for developing a disease, disorder or infection will actually develop the disease, disorder or infection.

As used herein, the term “subject” or “patient” can be used interchangeably and refer to any organism to which an IL-4 exposed M2 macrophage exosome or composition of the invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

As used herein, the terms “administering” and “administration” refer to any method of providing a disclosed IL-4 exposed M2 macrophage exosome or composition of the invention to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed exosome so as to treat a subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods of Making

Disclosed are method of producing IL-4 exposed M2 macrophage exosomes comprising culturing macrophage or macrophage precursor cells in the presence of IL-4 in culture media to thereby produce IL-4 exposed M2 macrophage exosomes, and isolating the IL-4 exposed M2 macrophage exosomes from the culture media, wherein the IL-4 exposed M2 macrophage exosomes are enriched with miR-21, miR-99a, miR-146b and miR378a. In some aspects, miR-21, miR-99a, miR-146b and miR378a are contributors to anti-inflammatory signaling.

In some aspects, the IL-4 is recombinant IL-4 cytokine. In some aspects, the IL-4 is recombinant human IL-4 cytokine.

In some aspects, the macrophage or macrophage precursor cells can be bone marrow derived macrophages (BMDMs) or THP (e.g. a human leukemia monocytic cell line) derived cells.

In some aspects, the cells are cultured with IL-4 for at least 12, 18, 24, 30, or 36 hours.

In some aspects, the method optionally further comprises, exposing the macrophage or macrophage precursor cells with PMA prior to culturing in the presence of IL4. In some aspects, a compound, such as PMA, can be used to differentiate a monocytic cell into a mature macrophage. For example, the THP1 cell line can be differentiated into mature macrophages with PMA.

In some aspects, about 6×10⁹ exosomes are secreted per million macrophage or macrophage precursor cells after 24 hours of culturing. In some aspects, about 1×10⁷ to 5×10¹⁰ exosomes are secreted per million macrophage or macrophage precursor cells after 24 hours of culturing.

In some aspects, the macrophage precursor cells are a monocyte cell line. In particular, in some aspects the monocyte cell line is a human monocyte cell line. In some aspects, the macrophage or macrophage precursor cells are M2-like macrophage or macrophage precursor cells. In some aspects, the M2-like macrophage cells are from a THP-1 cell line. In some aspects, the macrophage precursor cells are peripheral blood monocytes.

In some aspects, isolating exosomes from the culture media can be performed using any known technique. For example, isolating can comprise cushioned-density gradient ultracentrifugation.

In some aspects, the isolated IL-4 exposed M2 macrophage exosomes are between 80-110 nm. In some aspects, the isolated M2 macrophage exosomes are between 50-200 nm. In some aspects, the isolated IL-4 exposed M2 macrophage exosomes are about 90 nm.

### C. Methods of Reprogramming Macrophages

Disclosed are methods of reprogramming macrophages comprising exposing macrophages to IL-4 exposed M2 macrophage exosomes, wherein immune and/or metabolic properties are altered in the macrophages. In some aspects, IL-4 exposed M2 macrophages can polarize macrophages to an anti-inflammatory phenotype. In some aspects, IL-4 exposed M2 macrophages can reprogram the energy metabolism of macrophages.

In some aspects, the IL-4 exposed M2 macrophage exosomes are produced from the methods described herein.

In some aspects, the macrophages are exposed to IL-4 exposed M2 macrophage exosomes for at least 24 hours. In some aspects, the macrophages are exposed to IL-4 exposed M2 macrophage exosomes for at least 2, 4, 6, 8, 10, 12, 14, 16, 18, 20, 22, 24, 26, 28, 30, 32, 34, 36, 38, 40, 42, 44, 46, or 48 hours.

In some aspects, expression of one or more M2-associated genes is upregulated in the macrophages. For example, M2-associated genes that can be upregulated can be, but are not limited to, Arginase 1, Chitinase like-3, and Retnla.

In some aspects, expression of one or more M1-associated genes is downregulated in the macrophages. In some aspects, one or more pro-inflammatory markers are decreased in the macrophages. In some aspects, M1-associated genes are pro-inflammatory markers. Thus, in some aspects, the one or more M1-associated genes and/or pro-inflammatory markers or regulatory micro-RNA can be, but are not limited to, TNFα, IL-6 IL-1β, IFNγ, MCP1, or miRNA-33 and miR142a.

In some aspects, one or more anti-inflammatory markers including mRNA, protein and micro-RNA are increased in the macrophages. In some aspects, the one or more anti-inflammatory markers can be, but are not limited to, IL-10, CD206, CD163, or PPAR-γ, microRNA-199a/146b/378a. Thus, IL-4 exposed M2 macrophage produce exosomes that can reprogram recipient macrophages into macrophages that help fight inflammatory diseases or disorders in various tissues and organs.

In some aspects, oxidative phosphorylation (OXPHOS) in increased in the macrophages that take up IL4-exosomes. This increase in OXPHOS can help maintain metabolic homeostasis. In some aspects, mitochondrial activity is increased in the macrophages.

Further signs of cellular metabolism of the macrophages being altered by IL-4 exposed M2 macrophage exosomes is that in some instances, the size of lipid droplets in the macrophages is reduced. In some aspects, the size and density of the lipid droplets can be reduced.

In some aspects, IL-4 exposed M2 macrophages promote lipophagy that contributes to the utilization of cellular lipids as a source of energy to drive oxidative phosphorylation. In some aspects, expression of one or more genes associated with lipophagy is increased in the macrophages. In some aspects, the one or more genes associated with lipophagy are Ulk1, Pnpla2, Lipe, Map11c3a, Map11c3b and Cpt1a.

In some aspects, expression of one or more genes associated with cellular lipid synthesis is decreased in the macrophages. In some aspects, the one or more genes associated with cellular lipid synthesis are Srebf1 and Srebf2, along with the mater regulatory microRNA responsible for cellular lipid and energy storage, miR-33.

In some aspects, levels of one or more of microRNA-99a, 146a/b, and 378a are increased in the macrophages that take up IL4-THP1-exosomes. In some aspects, levels of microRNA-33-5p and microRNA-142a are also decreased in the macrophages. Collectively, such cellular signaling properties caused by IL-4 exposed M2 macrophage exosome can result in robust cellular reprograming in the recipient macrophages.

In some aspects, as disclosed herein, an increase in a marker or gene can mean a 5, 10, 15, 20, 25, 30, 35, 40, 45, 50% or higher increase in expression compared to the result of a macrophage not exposed to an IL-4 exposed M2 macrophage exosome. In some aspects, an increase in activity can mean a 5, 10, 15, 20, 25, 30, 35, 40, 45, 50% or higher increase in activity compared to the result of a macrophage not exposed to an IL-4 exposed M2 macrophage exosome. In some aspects, a decrease in size (e.g. of lipid droplets) can mean a 5, 10, 15, 20, 25, 30, 35, 40, 45, 50% or higher decrease in size compared to the result of a macrophage not exposed to an IL-4 exposed M2 macrophage exosome. In some aspects, an increase in activity can be a 2 fold, 3 fold, 4 fold or higher increase in activity compared to the result of a macrophage not exposed to an IL-4 exposed M2 macrophage exosome. In some aspects, a decrease in size can be a 2 fold, 3 fold, 4 fold or higher decrease in size compared to the result of a macrophage not exposed to an IL-4 exposed M2 macrophage exosome.

### D. Methods of Reprogramming Adipocytes

Disclosed are methods of reprogramming primary adipocytes comprising exposing adipocytes to IL-4 exposed M2 macrophage exosomes, wherein immune and/or metabolic properties are altered in the adipocytes.

In some aspects, the IL-4 exposed M2 macrophage exosomes are produced from the methods described herein.

In some aspects, the adipocytes are exposed to IL-4 exposed M2 macrophage exosomes for at least 24 hours. In some aspects, the adipocytes are exposed to IL-4 exposed M2 macrophage exosomes for at least 2, 4, 6, 8, 10, 12, 14, 16, 18, 20, 22, 24, 26, 28, 30, 32, 34, 36, 38, 40, 42, 44, 46, or 48 hours.

In some aspects, expression of the master regulatory gene PPARγ and the glucose transporter GLUT4 are increased in the adipocytes. In some aspects, GLUT4 is the target gene of PPARγ and therefore an increase in PPARγ results in an increase in GLUT4.

In some aspects, expression of one or more genes associated with cellular lipid synthesis is decreased in the adipocytes. In some aspects, the one or more genes associated with cellular lipid synthesis are Srebf1 and Srebf2, along with microRNA-33.

In some aspects, oxidative phosphorylation (OXPHOS) in increased in the adipocytes. This increase in OXPHOS can help maintain metabolic homeostasis. In some aspects, mitochondrial activity is increased in the adipocytes.

In some aspects, IL-4 exposed M2 macrophage exosomes can improve metabolic activity of adipocytes by modulating cellular microRNA. In some aspects, levels of one or more of microRNA-21, microRNA99a-5p, 146b-5p, and 378a-3p are increased in the adipocytes. In some aspects, levels of microRNA-33-5p are decreased in the adipocytes.

Further signs of cellular metabolism of the adipocytes being altered by IL-4 exposed M2 macrophage exosomes is that in some instances, the size of lipid droplets in the adipocytes is reduced. In some aspects, the size and density of the lipid droplets can be reduced.

In some aspects, wherein the adipocytes are white adipocytes. In some aspects, the white adipocytes undergo beiging. In some aspects, expression of genes associated with adipose tissue beiging are upregulated. In some aspects, genes associated with adipose tissue beiging can be, but are not limited to, Ucp1, Ppargc1a, Tbx1, Dio2, Zfp516, Prdm16, Prdm16, and Slc25a25. In some aspects, miRNA-33, which is recognized as a central driver of adipose tissue dysfunction and excessive lipid and energy storage, can also be reduced in adipocytes exposed to IL-4 exposed M2 macrophage exosomes.

### E. Methods of Increasing Anti-Inflammatory Markers

Disclosed are methods of increasing anti-inflammatory markers in a subject in need thereof comprising administering to the subject in need thereof a therapeutically effective amount of IL-4 exposed M2 macrophage exosomes. In some aspects, a subject in need thereof is a subject having an infection, or an inflammatory condition or disease. In some aspects, a subject in need thereof is a subject with inflammation in the lungs. In some aspects, a subject in need thereof is a subject having any conditions associated with either chronic or acute inflammation, such as but not limited to, acute myocardial infarction or acute kidney injury, or acute brain inflammatory reactions such as stroke.

In some aspects, IL4-exposed M2 macrophage exosomes can increase the levels of IL-10 and adiponectin in the blood of a subject administered an IL4-exposed M2 macrophage exosome.

In some aspects, pro-inflammatory markers are decreased simultaneously with anti-inflammatory markers being increased. For example, in some aspects, leptin, a pro-inflammatory marker, can be reduced in the blood of subjects administered IL-4 exposed M2 macrophage exosomes.

In some aspects, the IL-4 exposed M2 macrophage exosomes decrease inflammatory cells. In some aspects, the decrease in inflammatory cells is due to a decrease in expression of pro-inflammatory cytokines. Thus, in some aspects, the IL-4 exposed M2 macrophage exosomes decrease expression of pro-inflammatory cytokines. In some aspects, the pro-inflammatory cytokine can be, but is not limited to TNFα, IL-6 IL-1β, IFNγ, MCP1 or leptin. In some aspects, all of these cytokines can be reduced in the circulation of obese diabetic mice treated with the IL-4 exposed M2 macrophage exosomes

In some aspects, the IL-4 exposed M2 macrophage exosomes increase expression of anti-inflammatory cytokines. In some aspects, the one or more anti-inflammatory markers can be, but are not limited to, IL-10, CD206, CD163, PPAR-γ, or adiponectin. In some aspects, the combination of a decrease in pro-inflammatory cytokines and an increase in anti-inflammatory cytokines plays a role in the treatment of the disclosed diseases, disorders, or infections.

In some aspects, the IL-4 exposed M2 macrophage exosomes increase M2-associated markers. For example, M2-associated markers that can be upregulated can be, but are not limited to, Arginase 1, Chitinase like-3, and Retnla. In some aspects, the enrichment of M2 macrophages in tissues and organs of mice can result in the resolution of inflammatory reactions caused by metabolic excess such as in obesity and diabetes or in acute and chronic inflammation. In some aspects, this can promote the stabilization and regression of coronary atherosclerosis, suppress cardiac, lung, and brain inflammation, and control inflammation and organ disfunctions in compartments that include but are not limited to the liver, adipose tissue, kidkey, brain, and pancreas.

### F. Methods of Treating Diseases or Disorders

Disclosed are methods of treating diseases or disorders in a subject in need thereof comprising administering to the subject in need thereof a therapeutically effective amount of M2 macrophage exosomes to promote the resolution of inflammation and improve metabolic tissue function. For example, the disease or disorder can be diabetes, cardiometabolic disease, myocardial infarction, stroke, pancreatitis, chronic inflammatory disorder, septicemia, lethargy, acute coronary syndrome, or pulmonary infection.

Disclosed are methods of treating type II diabetes in a subject in need thereof comprising administering to the subject in need thereof a therapeutically effective amount of M2 macrophage exosomes.

Disclosed are methods of treating a cardiometabolic disease in a subject in need thereof comprising administering to the subject in need thereof a therapeutically effective amount of M2 macrophage exosomes. In some aspects, cardiometabolic disease can be clinical complications of type II diabetes, such as hyperglycemia and atherosclerosis cardiovascular disease resulting in myocardial infarction and stroke.

Disclosed are methods of treating a chronic inflammatory disorder in a subject in need thereof comprising administering to the subject in need thereof a therapeutically effective amount of M2 macrophage exosomes. In some aspects, IL4-exposed M2 macrophage exosomes can reduce the levels of pro-inflammatory cytokines. In some aspects, the pro-inflammatory cytokines can be, but are not limited to, IL-1β, TNF-α, Interferon-γ, IL-6. In some aspects, IL4-exposed M2 macrophage exosomes can increase anti-inflammatory markers. For example, IL-10 and adiponectin can be increased by IL4-exposed M2 macrophage exosomes.

Disclosed are methods of treating septicemia in a subject in need thereof comprising administering to the subject in need thereof a therapeutically effective amount of IL-4 exposed M2 macrophage exosomes. In some aspects, IL-4 exposed M2 macrophage exosomes can reduce inflammation by reducing leukocyte numbers. In some aspects, IL-4 exposed M2 macrophage exosomes can reduce IL-6 which can prevent fever and acute organ failure often found in acute sepsis.

Disclosed are methods of treating lethargy in a subject in need thereof comprising administering to the subject in need thereof a therapeutically effective amount of IL-4 exposed M2 macrophage exosomes. In some aspects, the lethargy can be from sepsis. As mentioned above, it is not clear but very likely. In some aspects, IL-4 exposed M2 macrophage exosomes can reduce IL-6 which can prevent fever, and thus lethargy, in response to a bacterial infection such as sepsis. Data has shown that IL-6 and ILI-beta can be controlled by IL-4 exposed M2 macrophage exosomes in mice that are treated with LPS, a chemical that serves as a surrogate model of acute bacterial sepsis.

Disclosed are methods of treating or ameliorating symptoms of acute coronary syndrome by administering to the subject in need thereof a therapeutically effective amount of IL-4 exposed M2 macrophage exosomes. In some aspects, the acute coronary syndrome can include, but is not limited to, coronary atherosclerosis rupture, myocardial infarction and stroke. Thus, disclosed are methods of reducing the risk of coronary atherosclerosis rupture, myocardial infarction and stroke

Disclosed are methods of treating a pulmonary infection and inflammation in a subject in need thereof comprising administering to the subject in need thereof a therapeutically effective amount of IL-4 exposed M2 macrophage exosomes. In some aspects, the pulmonary infection is due to infection of a viral pathogen. In some aspects, the pulmonary infection is due to infection of a viral pathogen or bacterial pathogen. In some aspects, the viral pathogen can be, but is not limited to, SARS-COV-2, influenza, respiratory syncytial virus (RSV), rhinovirus or adenovirus. In some aspects, the bacterial pathogen can be, but is not limited to, Streptococcus pneumoniae, Haemophilus influenzae, Staphylococcus aureus, Group A streptococci, Moraxella catarrhalis, anacrobes, and aerobic gram-negative bacteria.

In some aspects, the IL-4 exposed M2 macrophage exosomes administered to the subject are produced from the one of the methods of producing disclosed herein.

In some aspects, the IL-4 exposed M2 macrophage exosomes increase OXPHOS in recipient cells of the subject.

In some aspects, the IL-4 exposed M2 macrophage exosomes enhance insulin-stimulated glucose uptake in the subject. In some aspects, enhanced insulin-stimulated glucose uptake in the subject is important to tissues and cells responsive to insulin. Thus, in some aspects, the IL-4 exposed M2 macrophage exosomes can increase insulin sensitivity in cells and tissues, such as white adipose tissue, resulting in beiging caused by increased biogenesis of mitochondria that improve oxidative phosphorylation. In some aspects, this can also occur in other cells sensitive to insulin such as skeletal muscle cells and liver hepatocytes that can improve metabolic activity in the compartments.

In some aspects, the IL-4 exposed M2 macrophage exosomes increase OXPHOS in cells of the subject. In some aspects, the IL-4 exposed M2 macrophage exosomes upregulate lipophagy in cells of the subject. In some aspects, increased lipophagy and OXPHOS are associated with improvement of the health of white adipose tissue. In some aspects, an increase in OXPHOS can improve the cells capacity to utilize biofuels such as lipids, preventing an accumulation of lipids in cells. In some aspects, increased OXPHOS can also reduce the process of glycolysis and the excessive use of glucose for energy production thereby reducing the levels of reactive oxygen radicals produced in the cell that are recognized to cause cellular stress, senescence and premature cellular death.

In some aspects, the IL-4 exposed M2 macrophage exosomes increase production of adiponectin by adipocytes in the subject. In some aspects, adiponectin is a homeostatic factor for regulating glucose levels, lipid metabolism, and insulin sensitivity through its anti-inflammatory, anti-fibrotic, and antioxidant effects.

In some aspects, the IL-4 exposed M2 macrophage exosomes increase phagocytic uptake of apoptotic cells by macrophages in the subject. In some aspects, the IL-4 exposed M2 macrophage exosomes increase efferocytosis in the subject. In some aspects, efferocytosis is a process that results from phagocytosis of apoptotic cells and the production of highly potent anti-inflammatory cytokines that contribute to the resolution of tissue inflammation. In some aspects, efferocytosis is a specialized form of phagocytosis that results in the resolution of inflammation due to the secretion of anti-inflammatory cytokines, including IL-10, by the macrophage. In some aspects, efferocytosis is active in stabilizing human coronary atherosclerosis and thereby reduces the risk of a plaque rupture and acute myocardial infarction. In some aspects, IL-4 exposed M2 macrophage exosomes differentially control and augment efferocytosis and thereby the resolution of vascular and cardiometabolic inflammation through their capacity to regulate cellular signaling and bioenergetic metabolism in recipient macrophages and adipocytes and through the production of IL-10 in the recipient cells.

In some aspects, adipocytes and/or macrophages are altered by the IL-4 exposed M2 macrophages. In some aspects, immune and/or metabolic properties are altered. In some aspects, the altering of adipocytes and/or macrophages results in improved OXPHOS due to increased mitochondria biogenesis and function in metabolizing biofuels such as neutral lipid stores via upreagulated Cpt1a. In some aspects, IL-4 exposed M2 macrophage exosomes decrease microRNA142a which leads to an increase in CPT1a. CPT1a can augment fatty acid transport into the mitochondria where it increases OXPHOS (see Phu et al. ApoE expression in macrophages communicates immunometabolic signaling that controls hyperlipidemia-driven hematopoiesis & inflammation via extracellular vesicles. Journal of Extracellular Vesicles 2023; 12:12345; incorporated by reference in its entirety herein). Thus, in some aspects, the increase in OXPHOS can be due to the decrease in microRNA142a.

In some aspects, the IL-4 exposed M2 macrophage exosomes decrease insulin resistance. In some aspects, decreasing insulin resistance can prevent the development or progression of type 2 diabetes.

In some aspects, the IL-4 exposed M2 macrophage exosomes decrease inflammatory cells. In some aspects, the decrease in inflammatory cells is due to a decrease in expression of pro-inflammatory cytokines. Thus, in some aspects, the IL-4 exposed M2 macrophage exosomes decrease expression of pro-inflammatory cytokines. In some aspects, the pro-inflammatory cytokine can be, but is not limited to TNFα, IL-6 IL-1β, IFNγ, MCP1 or leptin.

In some aspects, the IL-4 exposed M2 macrophage exosomes increase expression of anti-inflammatory cytokines. In some aspects, the one or more anti-inflammatory markers can be, but are not limited to, IL-10, CD206, CD163, PPAR-γ, or adiponectin. In some aspects, the combination of a decrease in pro-inflammatory cytokines and an increase in anti-inflammatory cytokines plays a role in the treatment of the disclosed diseases, disorders, or infections.

In some aspects, the IL-4 exposed M2 macrophage exosomes increase M2-associated markers. For example, M2-associated markers that can be upregulated can be, but are not limited to, Arginase 1, Chitinase like-3, and Retnla. In some aspects, the polarized form of a macrophage (i.e. M2 macrophage) is critical to allow it to effect tissue-repair activities and to prevent excessive inflammation that can cause tissue injury and organ dysfunction such as in cases of acute infection, myocardial infarction and stroke. In some aspects, M2 macrophages are involved in the active resolution of inflammation and the beneficial remodeling of tissues following an acute of chronic bout of inflammation, such as Myocardial Infarction and atherosclerosis, in response to aggressive plasma lipid management.

In some aspects, miRNA-33 can be reduced upon macrophage exposure to IL-4 exposed M2 macrophage exosomes. Thus, in some aspects, a reduction of miRNA-33 can be a target to improve certain diseases or disorders, such as cardiometabolic health.

In some aspects, exaggerated myelopoiesis or hematopoiesis are reduced or decreased in the subject. In some aspects, the number of monocytes and/or neutrophils in the circulation or the spleen of the subject are reduced. This decrease in myelopoiesis and/or hematopoiesis can help control inflammation by reducing pro-inflammatory cell types and pro-inflammatory cytokines. Furthermore, in some aspects, leukocyte recruitment in the subject's aorta, liver or adipose tissue is suppressed or decreased.

In some aspects, the IL-4 exposed M2 macrophage exosomes are administered intraperitoneally. In some aspects, the IL-4 exposed M2 macrophage exosomes can be administered using any known administration route. For example, the IL-4 exposed M2 macrophage exosomes can be administered intraperitoneally, intravenously, subcutaneously, intranasally, orally, dermally, or via inhalation.

In some aspects, there is a decrease in pulmonary leukocyte infiltration. This decrease can reduce inflammation in the lungs. Thus, disclosed are methods of reducing inflammation in the lungs of a subject in need thereof comprising administering to the subject in need thereof a therapeutically effective amount of IL-4 exposed M2 macrophage exosomes.

In some aspects, the treatment from the IL-4 exposed M2 macrophage exosomes comes from a shift in cellular energy metabolism in recipient cells that leads to increased mitochondrial respiration. In some aspects, OXPHOS is recognized as a more effective mode of energy production and makes use of mitochondria and the use of neutral lipid biofuels in the cells. In some aspects, this process produces metabolites that are used by the cell to thrive. In some aspects, this can also reduce the cell's need to use glycolysis and thereby prevents the oxidative free radicals that can damage the cell or the cells in proximity.

### G. Methods of Reducing Acute Pulmonary Leukocyte Infiltration

Disclosed are methods of reducing acute pulmonary leukocyte infiltration, in a subject in need thereof comprising administering to the subject in need thereof a therapeutically effective amount of M2 macrophage exosomes. In some aspects, a subject in need thereof is a subject having a respiratory infection, condition or disease. In some aspects, a subject in need thereof is a subject with inflammation in the lungs. In some aspects, the inflammation in the lungs can be from a bacterial, viral or allergic infection/reactions.

In some aspects, the IL-4 exposed M2 macrophage exosomes administered to the subject are produced from one of the methods of producing disclosed herein.

In some aspects, the IL-4 exposed M2 macrophage exosomes increase OXPHOS in cells of the subject.

In some aspects, the IL-4 exposed M2 macrophage exosomes enhance insulin-stimulated glucose uptake in the subject. In some aspects, enhanced insulin-stimulated glucose uptake in the subject can help to control blood glucose levels and also eliminate fat storage as the lipids are consumed in the mitochondria. In some aspects, this effect can improve the severity of type II diabetes in obese mice that consume a diet rich in fat and carbohydrate.

In some aspects, the IL-4 exposed M2 macrophage exosomes increase OXPHOS in cells of the subject. In some aspects, the IL-4 exposed M2 macrophage exosomes upregulate lipophagy in cells of the subject. In some aspects, increased lipophagy and OXPHOS are associated with improvement of the health of white adipose tissue. In some aspects, an increase in OXPHOS can improve the cells capacity to utilize biofuels such as lipids, preventing an accumulation of lipids in cells. In some aspects, increased OXPHOS can also reduce the process of glycolysis and the excessive use of glucose for energy production thereby reducing the levels of reactive oxygen radicals produced in the cell that are recognized to cause cellular stress, senescence and premature cellular death.

In some aspects, the IL-4 exposed M2 macrophage exosomes increase production of adiponectin by adipocytes in the subject. In some aspects, adiponectin is a homeostatic factor for regulating glucose levels, lipid metabolism, and insulin sensitivity through its anti-inflammatory, anti-fibrotic, and antioxidant effects.

In some aspects, the IL-4 exposed M2 macrophage exosomes increase phagocytic uptake of apoptotic cells by macrophages in the subject. In some aspects, the IL-4 exposed M2 macrophage exosomes increase efferocytosis in the subject. In some aspects, efferocytosis is a process that results from phagocytosis of apoptotic cells and the production of highly potent anti-inflammatory cytokines that contribute to the resolution of tissue inflammation. In some aspects, efferocytosis is a specialized form of phagocytosis that results in the resolution of inflammation due to the secretion of anti-inflammatory cytokines, including IL-10, by the macrophage. In some aspects, efferocytosis is active in stabilizing human coronary atherosclerosis and thereby reduces the risk of a plaque rupture and acute myocardial infarction. In some aspects, IL-4 exposed M2 macrophage exosomes differentially control efferocytosis and thereby the resolution of vascular and cardiometabolic inflammation through their capacity to regulate cellular signaling and bioenergetic metabolism in recipient macrophages and adipocytes.

In some aspects, adipocytes and/or macrophages are altered by the IL-4 exposed M2 macrophages. In some aspects, immune and/or metabolic properties are altered. In some aspects, the altering of adipocytes and/or macrophages

In some aspects, the IL-4 exposed M2 macrophage exosomes decrease insulin resistance. In some aspects, decreasing insulin resistance can prevent the development or progression of type 2 diabetes.

In some aspects, the IL-4 exposed M2 macrophage exosomes decrease inflammatory cells. In some aspects, the decrease in inflammatory cells is due to a decrease in expression of pro-inflammatory cytokines. Thus, in some aspects, the IL-4 exposed M2 macrophage exosomes decrease expression of pro-inflammatory cytokines. In some aspects, the pro-inflammatory cytokine can be, but is not limited to TNFα, IL-6 IL-1β, IFNγ, MCP1 or leptin.

In some aspects, the IL-4 exposed M2 macrophage exosomes increase expression of anti-inflammatory cytokines. In some aspects, the one or more anti-inflammatory markers can be, but are not limited to, IL-10, CD206, CD163, PPAR-γ, or adiponectin. In some aspects, the combination of a decrease in pro-inflammatory cytokines and an increase in anti-inflammatory cytokines plays a role in the treatment of the disclosed diseases, disorders, or infections.

In some aspects, the IL-4 exposed M2 macrophage exosomes increase M2-associated markers. For example, M2-associated markers that can be upregulated can be, but are not limited to, Arginase 1, Chitinase like-3, and Retnla. In some aspects, the polarized form of a macrophage (i.e. M2 macrophage) is critical to allow it to effect tissue-repair activities and to prevent excessive inflammation that can cause tissue injury and organ dysfunction such as in cases of acute infection, myocardial infarction and stroke. In some aspects, M2 macrophages are involved in the active resolution of inflammation and the beneficial remodeling of tissues following an acute of chronic bout of inflammation, such as Myocardial Infarction and atherosclerosis, in response to aggressive plasma lipid management.

In some aspects, miRNA-33 can be reduced upon macrophage exposure to IL-4 exposed M2 macrophage exosomes. Thus, in some aspects, a reduction of miRNA-33 can be a target to improve certain diseases or disorders, such as cardiometabolic health.

In some aspects, exaggerated myelopoiesis or hematopoiesis are reduced or decreased in the subject. In some aspects, the number of monocytes and/or neutrophils in the circulation or the spleen of the subject are reduced. This decrease in myelopoiesis and/or hematopoiesis can help control inflammation by reducing pro-inflammatory cell types and pro-inflammatory cytokines. Furthermore, in some aspects, leukocyte recruitment in the subject's aorta, liver or adipose tissue is suppressed or decreased.

In some aspects, the IL-4 exposed M2 macrophage exosomes are administered intraperitoneally. In some aspects, the IL-4 exposed M2 macrophage exosomes can be administered using any known administration route. For example, the IL-4 exposed M2 macrophage exosomes can be administered intraperitoneally, intravenously, subcutaneously, intranasally, orally, dermally or via inhalation.

### H. Methods of Enhancing Oxidative Phosphorylation

Disclosed are methods of enhancing oxidative phosphorylation (OXPHOS) in a cell of a subject in need thereof comprising administering to the subject in need thereof a therapeutically effective amount of IL-4 exposed M2 macrophage exosomes. In some aspects, the subject in need thereof is a subject having an infection, disease, or disorder. In some aspects, the subject in need thereof is a subject having diabetes.

In some aspects, the cell is an adipocyte or macrophage.

In some aspects, enhancing OXPHOS results in the production of ATP. In some aspects, the production of ATP helps the cells function better as it is a source of cellular energy.

In some aspects, increased OXPHOS can improve white adipose tissue health which can be used for the control of insulin resistance and treatment of type 2 diabetes.

In some aspects, IL-4 exposed M2 macrophage exosomes decrease microRNA142a which leads to an increase in CPT1a. In some aspects, CPT1a can augment fatty acid transport into the mitochondria where it increases OXPHOS. Thus, in some aspects, the increase in OXPHOS can be due to the decrease in microRNA142a.

### I. Methods of Inducing White Adipose Tissue Beiging

Disclosed are methods of inducing white adipose tissue beiging in a subject in need thereof comprising administering to the subject in need thereof a therapeutically effective amount of IL-4 exposed M2 macrophage exosomes. In some aspects, a subject in need thereof is a subject having diabetes or obesity.

In some aspects, the IL-4 exposed M2 macrophage exosomes administered to the subject are produced from the one of the methods of producing disclosed herein.

In some aspects, the IL-4 exposed M2 macrophage exosomes are administered intraperitoneally. In some aspects, the IL-4 exposed M2 macrophage exosomes can be administered using any known administration route. For example, the IL-4 exposed M2 macrophage exosomes can be administered intraperitoneally, intravenously, subcutaneously, intranasally, orally, dermally, or via inhalation.

In some aspects, inducing beiging of white adipose tissue enhances energy expenditure by reducing lipids stored within adipose tissue. Thus, inducing beiging in white adipose tissue can be used to combat the growing epidemic of obesity.

In some aspects, the induction of beiging occurs due to the generation of more mitochondria that foster OXPOHOS. In some aspects, this effect promotes the use of lipid stores in adipocytes and also stimulates the uptake of glucose from the circulation. These sources of energy are then metabolized by the mitochondria and can result in “thermogenesis” resulting from the activity of a protein called “UCP-1” that uncouples the electron chain in the mitochondria and produces heat to warm the body.

### J. Methods of Inducing Lipophagy

Disclosed are methods of inducing lipophagy in a cell of a subject comprising administering to the subject in need thereof a therapeutically effective amount of IL-4 exposed M2 macrophage exosomes. In some aspects, the size and density of lipid droplets is reduced in the subject.

In some aspects, the IL-4 exposed M2 macrophage exosomes administered to the subject are produced from the one of the methods of producing disclosed herein.

In some aspects, genes associated with lipophagy included, but not limited to, Ulk1, Pnpla2, Lipe, Map1lc3a, Map1lc3b, and Cpt1a can be increased in response to administration of IL-4 exposed M2 macrophage exosomes.

In some aspects, the IL-4 exposed M2 macrophage exosomes are administered intraperitoneally. In some aspects, the IL-4 exposed M2 macrophage exosomes can be administered using any known administration route. For example, the IL-4 exposed M2 macrophage exosomes can be administered intraperitoneally, intravenously, subcutaneously, intranasally, orally, dermally, or via inhalation.

Thus, in some aspects, treating with IL-4 exposed M2 macrophage exosomes can regulate cellular lipid metabolism, including by inducing lipophagy.

In some aspects, a subject in need thereof is a subject that is overweight.

In some aspects, the induction of lipophagy benefits numerous cell types beyond the adipocyte. In some aspects, induction of lipophagy in immune cells can program anti-inflammatory and tissue reparative properties that can promote the resolution of inflammation in numerous compartments and organ systems including but not limited to the artery vessel wall, as well as the heart and kidney following acute or chronic ischemic injury.

### K. Methods of Suppressing Cardiac or Pulmonary Inflammation

Disclosed are methods of suppressing cardiac or pulmonary inflammation in a subject in need thereof comprising administering to the subject in need thereof a therapeutically effective amount of IL-4 exposed M2 macrophage exosomes. In some aspects, a subject in need thereof is a subject having a cardiac or respiratory infection, condition or disease. In some aspects, a subject in need thereof is a subject with inflammation in the heart or lungs. In some aspects, a subject in need thereof is a subject experiencing inflammation in organ systems such as the liver, gut, pancreas, kidney and brain.

In some aspects, the pulmonary inflammation is due to pulmonary infection. In some aspects, the pulmonary infection is due to infection of a viral pathogen or bacterial pathogen. In some aspects, the viral pathogen can be, but is not limited to, SARS-COV-2, influenza, respiratory syncytial virus (RSV), rhinovirus or adenovirus. In some aspects, the bacterial pathogen can be, but is not limited to, Streptococcus pneumoniae, Haemophilus influenzac, Staphylococcus aureus, Group A streptococci, Moraxella catarrhalis, anaerobes, and aerobic gram-negative bacteria.

In some aspects, IL-4 exposed M2 macrophage exosomes increase PPAR-γ in the subject. PPAR-γ is an important transcription factor that, in some instances, reduces macrophage activation by increasing expression of genes such as ApoE and ABCA1 that protect against cardiovascular inflammation.

In some aspects, the IL-4 exposed M2 macrophage exosomes decrease inflammatory cells. In some aspects, the decrease in inflammatory cells is due to a decrease in expression of pro-inflammatory cytokines. Thus, in some aspects, the IL-4 exposed M2 macrophage exosomes decrease expression of pro-inflammatory cytokines. In some aspects, the pro-inflammatory cytokine can be, but is not limited to TNFα, IL-6 IL-1β, IFNγ, MCP1 or leptin.

In some aspects, the IL-4 exposed M2 macrophage exosomes increase expression of anti-inflammatory cytokines. In some aspects, the one or more anti-inflammatory markers can be, but are not limited to, IL-10, CD206, CD163, PPAR-γ, or adiponectin. In some aspects, the combination of a decrease in pro-inflammatory cytokines and an increase in anti-inflammatory cytokines plays a role in the treatment of the disclosed diseases, disorders, or infections.

In some aspects, the IL-4 exposed M2 macrophage exosomes increase M2-associated markers. For example, M2-associated markers that can be upregulated can be, but are not limited to, Arginase 1, Chitinase like-3, and Retnla. As stated throughout, in some aspects, the value of fostering M2 macrophage polarization via IL-4 exposed M2 macrophage exosomes rests with the intrinsic capacity for this cell type to promote the resolution of inflammation and effect tissue repair in numerous organ and tissue compartments. In some aspects, benefits of fostering M2 macrophage activity in the adipose tissue is recognized to improve insulin sensitivity and prevent diabetes, while M2 macrophage activity in the cardiovascular system is recognized to foster atherosclerosis lesion stabilization and regression to prevent plaque rupture and acute myocardial infarction and stroke. Similarly, the presence of M2 macrophage activity in the heart and kidney has been documented to benefit against the onset of heart and kidney failure.

### L. Compositions

Disclosed herein are IL-4 exposed M2 macrophage exosomes. In some aspects, the disclosed IL-4 exposed M2 macrophage exosomes are produced by the methods disclosed herein. Disclosed are recombinant exosomes enriched with microRNA including miR-21, miR-99a, miR-146b and miR378a by a minimum of 3 to 4 fold over control exosomes, such as macrophage derived exosomes that were not exposed to PMA or IL-4.

In some aspects, the disclosed IL-4 exposed M2 macrophage exosomes or recombinant exosomes are membrane bound particles and typically range in diameter from about 15 nm to about 95 nm in diameter, including about 15 nm to about 20 nm, 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 95 nm, and overlapping ranges thereof. In some aspects, the disclosed IL-4 exposed M2 macrophage exosomes or recombinant exosomes are larger (e.g., those ranging from about 140 to about 210 run, including about 140 nm to about 150 nm, 150 nm to about 160 run, 160 nm to about 170 run, 170 nm to about 180 nm, 180 nm to about 190 run, 190 nm to about 200 run, 200 nm to about 210 nm, and overlapping ranges thereof).

Alternative nomenclature can also often be used to refer to the disclosed IL-4 exposed M2 macrophage exosomes or recombinant exosomes. Thus, as used herein the term “exosome” shall be given its ordinary meaning and may also include terms including microvesicles, epididimosomes, argosomes, exosome-like vesicles, microparticles, promininosomes, prostasomes, dexosomes, texosomes, dex, tex, archeosomes and oncosomes. Unless otherwise indicated herein, each of the foregoing terms shall also be understood to include engineered high-potency varieties of each type of exosome. Exosomes are secreted by a wide range of mammalian cells and are secreted under both normal and pathological conditions. Exosomes, in some embodiments, function as intracellular messengers by virtue of carrying mRNA, miRNA or other contents from a first cell to another cell (or plurality of cells). In several embodiments, exosomes are involved in blood coagulation, immune modulation, metabolic regulation, cell division, and other cellular processes. Because of the wide variety of cells that secret exosomes, in several embodiments, exosome preparations can be used as a diagnostic tool (e.g., exosomes can be isolated from a particular tissue, evaluated for their nucleic acid or protein content, which can then be correlated to disease state or risk of developing a disease).

In some aspects, the disclosed IL-4 exposed M2 macrophage exosomes or recombinant exosomes are isolated from cellular preparations by methods comprising one or more of filtration, centrifugation, antigen-based capture and the like. For example, in several embodiments, a population of cells (e.g. macrophage or macrophage precursor cells) are grown in culture are collected and pooled. In some aspects, monolayers of cells are used, in which case the cells are optionally treated in advance of pooling to improve cellular yield (e.g., dishes are scraped and/or enzymatically treated with an enzyme such as trypsin to liberate cells). In some aspects, cells grown in culture under standard cell culture conditions are exposed to serum-free medium under hypoxic condition overnight, and conditioned media containing exosomes are collected. In some embodiments, the hypoxic condition includes about 15%, about 12%, about 10%, about 9%, about 8%, about 7%, about 6%, about 5%, about 4%, about 3%, about 2%, about 1%, O₂ or less, or a percentage of O₂ in a range defined by any two of the preceding values. In some embodiments, the hypoxic condition includes 2% O₂/5% CO₂ at 37° C. In some embodiments, the cells exposed to hypoxic condition recover in complete serum under standard culture conditions for about 24, about 36, about 48, about 60, about 72 hours or more, or a time interval in a range defined by any two of the preceding values, and are then re-exposed to hypoxic condition to generate condition media. In some embodiments, cells are cycled between hypoxic and standard cell culture conditions for 1, 2, 3, 4, 5, 6 or more times. In several embodiments, cells grown in suspension are used. The pooled population is then subject to one or more rounds of centrifugation (in several embodiments ultracentrifugation and/or density centrifugation is employed) in order to separate the exosome fraction from the remainder of the cellular contents and debris from the population of cells. In some embodiments, centrifugation need not be performed to harvest exosomes. In several embodiments, pre-treatment of the cells is used to improve the efficiency of exosome capture. For example, in several embodiments, agents that increase the rate of exosome secretion from cells are used to improve the overall yield of exosomes. In some embodiments, augmentation of exosome secretion is not performed. In some embodiments, size exclusion filtration is used in conjunction with, or in place of centrifugation, in order to collect a particular size (e.g., diameter) of exosome. In several embodiments, filtration need not be used. In still additional embodiments, exosomes (or subpopulations of exosomes are captured by selective identification of unique markers on or in the exosomes (e.g., transmembrane proteins)). In such embodiments, the unique markers can be used to selectively enrich a particular exosome population. In some embodiments, enrichment, selection, or filtration based on a particular marker or characteristic of exosomes is not performed.

Upon administration of the disclosed IL-4 exposed M2 macrophage exosomes or recombinant exosomes can fuse with the cells of a target tissue. As used herein, the term “fuse” shall be given its ordinary meaning and shall also refer to complete or partial joining, merging, integration, or assimilation of the exosome and a target cell. In several embodiments, the exosomes fuse with healthy cells of a target tissue. In some embodiments, the fusion with healthy cells results in alterations in the healthy cells that leads to beneficial effects on the damaged or diseased cells (e.g., alterations in the cellular or intercellular environment around the damaged or diseased cells). In some embodiments, the exosomes fuse with damaged or diseased cells. In some such embodiments, there is a direct effect on the activity, metabolism, viability, or function of the damaged or diseased cells that results in an overall beneficial effect on the tissue. In several embodiments, fusion of the exosomes with either healthy or damaged cells is not necessary for beneficial effects to the tissue as a whole (e.g., in some embodiments, the exosomes affect the intercellular environment around the cells of the target tissue). Thus, in several embodiments, fusion of the disclosed IL-4 exposed M2 macrophage exosomes or recombinant exosomes to another cell does not occur. In several embodiments, there is no cell-exosome contact, yet the exosomes still influence the recipient cells.

Also disclosed herein are compositions comprising the disclosed IL-4 exposed M2 macrophage exosomes or recombinant exosomes. In some aspects, the disclosed IL-4 exposed M2 macrophage exosomes or recombinant exosomes are produced by the methods disclosed herein.

Also disclosed herein are pharmaceutical compositions comprising the disclosed IL-4 exposed M2 macrophage exosomes or recombinant exosomes. In some aspects, the disclosed IL-4 exposed M2 macrophage exosomes or recombinant exosomes are produced by the methods disclosed herein.

The disclosed compositions can further comprise a pharmaceutically acceptable carrier.

### 1. Delivery of Compositions

In the methods described herein, delivery (or administration) of the disclosed IL-4 exposed M2 macrophage exosomes or recombinant exosomes or compositions disclosed herein to cells can be via a variety of mechanisms. As defined above, disclosed herein are compositions comprising any one or more of the disclosed IL-4 exposed M2 macrophage exosomes or recombinant exosomes and can also include a carrier such as a pharmaceutically acceptable carrier. For example, disclosed are pharmaceutical compositions, comprising the IL-4 exposed M2 macrophage exosomes disclosed herein and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl choline (DMPC), phosphate buffered saline or a multivesicular liposome. For example, PG: PC: Cholesterol: peptide or PC: peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically acceptable salt is used in the formulation to render the formulation isotonic. Other examples of the pharmaceutically acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed delivery techniques can be used not only for the disclosed compositions but also the disclosed IL-4 exposed M2 macrophage exosomes or recombinant exosomes.

In some aspects, the disclosed IL-4 exposed M2 macrophage exosomes or recombinant exosomes derived from cells (e.g., macrophage or macrophage precursor cells) are administered in combination with one or more additional agents. For example, in several embodiments, the disclosed IL-4 exposed M2 macrophage exosomes or recombinant exosomes are administered in combination with one or more proteins or nucleic acids derived from the disclosed IL-4 exposed M2 macrophage exosomes or recombinant exosomes (e.g., to supplement the exosomal contents). In some aspects, the cells from which the disclosed IL-4 exposed M2 macrophage exosomes or recombinant exosomes are isolated are administered in conjunction with the disclosed IL-4 exposed M2 macrophage exosomes or recombinant exosomes. In several embodiments, such an approach advantageously provides an acute and more prolonged duration of exosome delivery (e.g., acute based on the actual exosome delivery and prolonged based on the cellular delivery, the cells continuing to secrete exosomes post-delivery).

In some aspects, the disclosed IL-4 exposed M2 macrophage exosomes or recombinant exosomes are delivered in conjunction with a more traditional therapy, e.g., surgical therapy or pharmaceutical therapy. In some aspects, such combinations of approaches result in synergistic improvements in the viability and/or function of the target tissue. In some embodiments, the disclosed IL-4 exposed M2 macrophage exosomes or recombinant exosomes may be delivered in conjunction with a gene therapy vector (or vectors), nucleic acids (e.g., those used as siRNA or to accomplish RNA interference), and/or combinations of the disclosed IL-4 exposed M2 macrophage exosomes or recombinant exosomes derived from other cell types.

The dose of the disclosed IL-4 exposed M2 macrophage exosomes or recombinant exosomes administered, depending on the embodiment, can range from about 1.0×10⁵ to about 1.0×10⁹ exosomes, including about 1.0×10⁵ to about 1.0×10⁶, about 1.0×10⁶ to about 1.0×10⁷, about 1.0×10⁷ to about 5.0×10⁷, about 5.0×10⁷ to about 1.0×10⁸, about 1.0×10⁸ to about 2.0×10⁸, about 2.0×10⁸ to about 3.5×10⁸, about 3.5×10⁸ to about 5.0×10⁸, about 5.0×10⁸ to about 7.5×10⁸, about 7.5×10⁸ to about 1.0×10⁹, and overlapping ranges thereof. In certain embodiments, the exosome dose is administered on a per kilogram basis, for example, about 1.0×10⁵ exosomes/kg to about 1.0×10⁹ exosomes/kg. In additional embodiments, exosomes are delivered in an amount based on the mass of the target tissue, for example about 1.0×10⁵ exosomes/gram of target tissue to about 1.0×10⁹ exosomes/gram of target tissue. In several embodiments, exosomes are administered based on a ratio of the number of exosomes the number of cells in a particular target tissue, for example exosome: target cell ratio ranging from about 10⁹:1 to about 1:1, including about 10⁸:1, about 10⁷:1, about 10⁶:1, about 10⁵:1, about 10⁴:1, about 10³:1, about 10²:1, about 10:1, and ratios in between these ratios. In additional embodiments, exosomes are administered in an amount about 10-fold to an amount of about 1,000,000-fold greater than the number of cells in the target tissue, including about 50-fold, about 100-fold, about 500-fold, about 1000-fold, about 10,000-fold, about 100,000-fold, about 500,000-fold, about 750,000-fold, and amounts in between these amounts. If the exosomes are to be administered in conjunction with the concurrent therapy (e.g., cells that can still shed exosomes, pharmaceutical therapy, nucleic acid therapy, and the like) the dose of exosomes administered can be adjusted accordingly (e.g., increased or decreased as needed to achieve the desired therapeutic effect). Advantageously, the engineered high-potency exosomes disclosed herein allow for reduced doses of exosomes to be used, in several embodiments with enhanced therapeutic effects despite the lower dose.

### M. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising the disclosed IL-4 exposed M2 macrophage exosomes or recombinant exosomes. In some aspects, the kits can comprise the elements required to the disclosed IL-4 exposed M2 macrophage exosomes or recombinant exosomes. For example, the kits can comprise one or more of macrophage or macrophage precursor cells (e.g., THP1 cell line), recombinant IL-4, PMA, and culture media.

### EXAMPLES

### A. Example 1: IL-4-Polarized Human Macrophage Exosomes

THP1 cells are treated a little differently as they are first induced with a chemical called PMA (for 48 hours) allowing them to transform from a monocyte to a macrophage. As such, these cells are somewhat different than BMDM that are first exposed to m-CSF to allow than to differentiate The method for making the exosomes beginning with TPI-1 cells is described in Phu et al. IL-4 polarized human macrophage exosomes control cardiometabolic inflammation and diabetes in obesity, Mol Ther. 2022 Jun. 1; 30 (6): 2274-2297 incorporated herein by reference in its entirety.

The data provided herein, demonstrates that IL-4-polarized human macrophage exosomes are potent in controlling acute and chronic inflammatory diseases in the cardiopulmonary and cardiometabolic systems and exert tissue reparative properties in resolving inflammation in these tissues. The data indicate that they can be equally effective in controlling other forms of tissue and organ dysfunction as a consequence of inflammation, including neurodegenerative disorders caused by both acute and chronic neuroinflammation, ischemia, and trauma.

The exosomes are produced by exposing the human macrophage cell line THP-1, to the recombinant human IL-4 cytokine for a period of 24 hours. The conditioned cell culture medium is collected and exosomes identified as “THP1-IL4-exo” (also referred to as IL-4 exposed M2 macrophage exosomes) are purified.

When tested in-vitro, purified THP-IL4-exo display a dose responsive capacity to control energy metabolism and inflammatory signaling in cultured THP-1 macrophages and primary mouse macrophages. Furthermore, THP-IL4-exo enhance insulin-stimulated glucose uptake in cultured mouse 3T3-L1 adipocytes. They also induce lipophagy, drive oxidative phosphorylation (OXPHOS), cause the browning of cultured 3T3-L1 adipocytes and promote the production of adiponectin by these cells. Such biological properties are classically associated with the improvement of white adipose tissue health required for the control of insulin resistance and treatment of type 2 diabetes.

THP-IL4-exo are enriched with microRNA including miR-21, miR-99a, miR-146b and miR-378a that were previously identified as contributors to the anti-inflammatory signaling properties in a mouse model of hyperlipidemia and atherosclerosis.

When tested in-vivo through triweekly intra-peritoneal infusions into mice fed a high-fat Western diet, THP1-IL4-exo display potent properties in controlling key aspects of cardiometabolic diseases and type II diabetes. Furthermore, when infused into mice that develop occlusive coronary atherosclerosis and myocardial infarction, THP1-IL4-exo potently suppress cardiac inflammation and preserve cardiac function. Finally, when infused into mice injected with bacterial lipopolysaccharide (LPS), THP1-IL4-exo reduced both systemic and pulmonary inflammation as observed through reduced leukocyte number in bronchioalveolar lavage and by preventing lethargy in septic mice.

Cellular and molecular mechanisms to explain the mode of action of THP1-IL4-exo in their control of cardiometabolic inflammation and Type 2 diabetes are discussed below and throughout the Examples. Briefly, THP1-IL4-exo reduce exaggerated myelopoiesis and hematopoiesis caused by chronic hyperlipidemia, diabetic hyperglycemia and obesity in mice. This results in a profound reduction in the number of monocytes and neutrophils in the circulation and the spleen. The source of such inflammation control comes from a deceleration of hematopoiesis in these mice through as documented through reduced numbers of hematopoietic stem and progenitor cells in the bone marrow and spleen. The magnitude of systemic inflammation control exerted by THP1-IL4-exo is illustrated by the profound reduction in inflammatory cytokines in the circulation of these mice that include, IFN-gamma, IL-6, TNF-alpha, and IL-1beta. Furthermore, repeated infusions of THP1-IL4-exo potently suppresses leukocyte recruitment and inflammatory gene expression in the aorta, liver, and adipose tissue. Concomitantly, this results in reduced neutral lipid accumulation in these tissues.

At the molecular level, THP1-IL4-exo infusions into obese diabetic mice confer immunometabolic control by enhancing glucose disposal, mitochondrial respiration and OXPHOS, along with the browning also known as “beiging” of white adipose tissue and the secretion of adiponectin into plasma. Together, the observed effects of THP1-IL4-exo infusions prevent the onset of obesity-driven insulin resistance, hyperglycemia and type II diabetes in mice fed a Western high-fat diet.

Collectively, THP1-IL4-exo can be used in a treatment for chronic inflammatory disorders in the cardiopulmonary system, as well as in cardiovascular and cardiometabolic tissue including ischemic cardiac tissue, pulmonary inflammation, atherosclerosis, fatty liver, obesity, as well as insulin resistance and impaired glucose control in diabetes.

THP1-IL4-exo can also control both acute and chronic neuro-inflammation caused by ischemic and traumatic injury and their consequences in neurodegenerative diseases.

Based on their potent efficacy in controlling acute pulmonary leukocyte infiltration, septicemia, lethargy and premature death in mice injected with LPS, THP1-IL4-exo can be used as biologics to control chronic pulmonary inflammation in respiratory infections including viral pathogens such as SARS-COV-2.

### B. Example 4: IL-4 Polarized Human Macrophage Exosomes Control Cardiometabolic Inflammation & Diabetes in Obesity

### 1. Introduction

Cardiometabolic inflammatory disease and its associated complications are the leading causes of morbidity and mortality due to the increasing prevalence of diabetes. Risk factors contributing to its pathogenesis include obesity, insulin-resistance, dyslipidemia, and hypertension. Recent findings point to chronic, unresolved inflammation as a major contributor to the onset and progression of cardiometabolic disease and its complications. A hallmark of this inflammatory response includes an accumulation of pro-inflammatory M1-like macrophages in the liver, as well as in adipose and vascular tissues. A number of studies have identified the release of pro-inflammatory cytokines including tumor necrosis factor-alpha (TNF-α) and interleukin-1-beta (IL-1β) from tissue-associated M1-like macrophages as factors that further exacerbates local inflammation and inhibit insulin sensitivity. In contrast, anti-inflammatory M2-like macrophages have been shown to exert protective properties in the liver, as well as in adipose and vascular tissues. While polarizing adipose tissue macrophages to an M2-like phenotype protects high fat diet-fed mice against obesity-induced insulin resistance and induces beiging in white adipose tissues, key signaling factors that could be exploited to drive this beneficial phenomenon remain elusive.

Extracellular vesicles (EVs) including exosomes have recently been recognized as sources of intercellular communication in numerous disease states including inflammation and metabolic disease. EVs, including exosomes are increasingly reported to serve in modulating obesity beyond cytokines and other signaling factors. Specifically, exosomes produced by macrophages have been shown to control numerous disease states through the delivery of cargo including microRNA. Macrophages derived from obese adipose tissue and bone marrow (BMDM) exposed to lipopolysaccharide (LPS) have both been reported to display features of inflammatory M1 macrophages. Exosomes released by such M1-like macrophages are enriched in miR-155 that can be communicated to suppress insulin sensitivity in cultured adipocytes and adipose tissue when injected into obese mice fed a high fat diet. Recent findings that examined exosomes derived from BMDM cultured with elevated glucose levels revealed the production of exosomes with altered microRNA cargo including high levels of miR-486-5p that downregulate the expression of Abca1 in macrophages. Furthermore, such “high-glucose” macrophage exosomes were found to drive hematopoiesis and accelerate atherosclerosis when infused into Apoe^{-/-} mice. Collectively, results of these studies support exosomes produced by macrophages exposed to inflammatory cytokines and metabolic stress as contributors to cardiometabolic disease.

In contrast, increasing evidence points to exosomes produced by alternatively activated M2-like macrophages as contributors to inflammation control. Indeed, results of the study that examined exosomes produced by M2-like BMDM exposed to IL-4 revealed their capacity to control hematopoiesis, inflammation, and atherosclerosis in Apoe^{-/-} mice. The findings showed that they did so in part through the delivery of a cluster of microRNA cargo including miR-99a/146b/378a that controlled NF-κB inflammatory activity. Results of a more recent study of M2-like BMDM exosomes further revealed their ability to improve insulin sensitivity and glucose tolerance in adipose tissue of obese mice through the delivery of miR-690. Taken together, these findings support M2-like macrophage exosomes as promising therapeutics for the control of cardiometabolic inflammatory disease. This study aimed at developing translational opportunities for M2-like macrophage exosomes, by investigating properties conferred by those derived from IL-4-exposed human THP-1 macrophages.

### 2. Results

i. Functional Studies of Exosomes Produced by Human THP-1 Macrophages

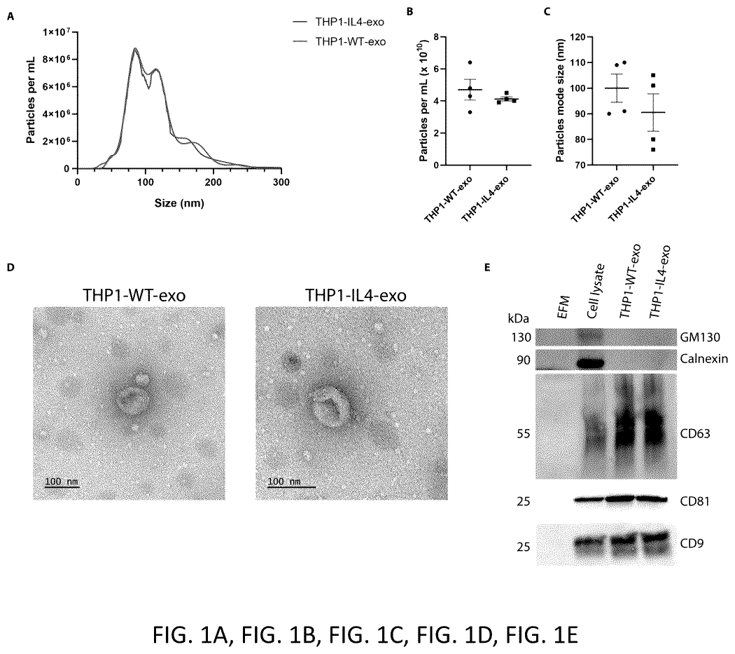

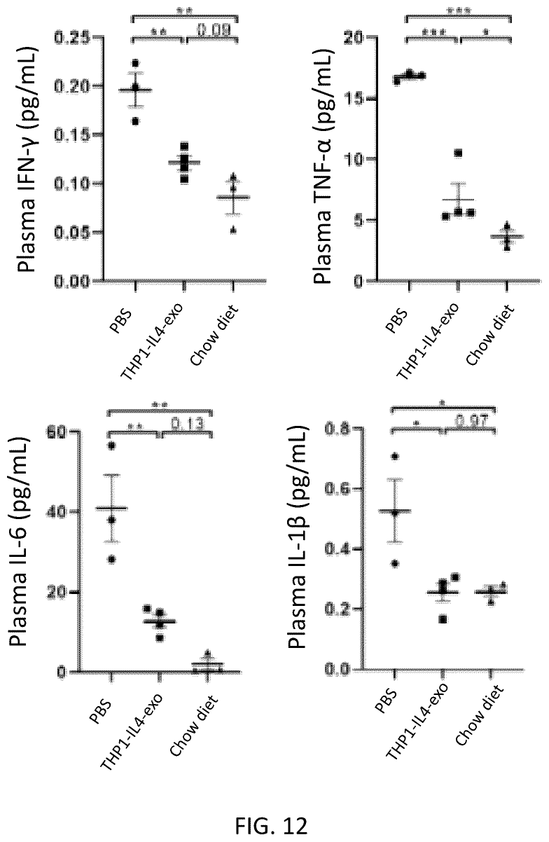

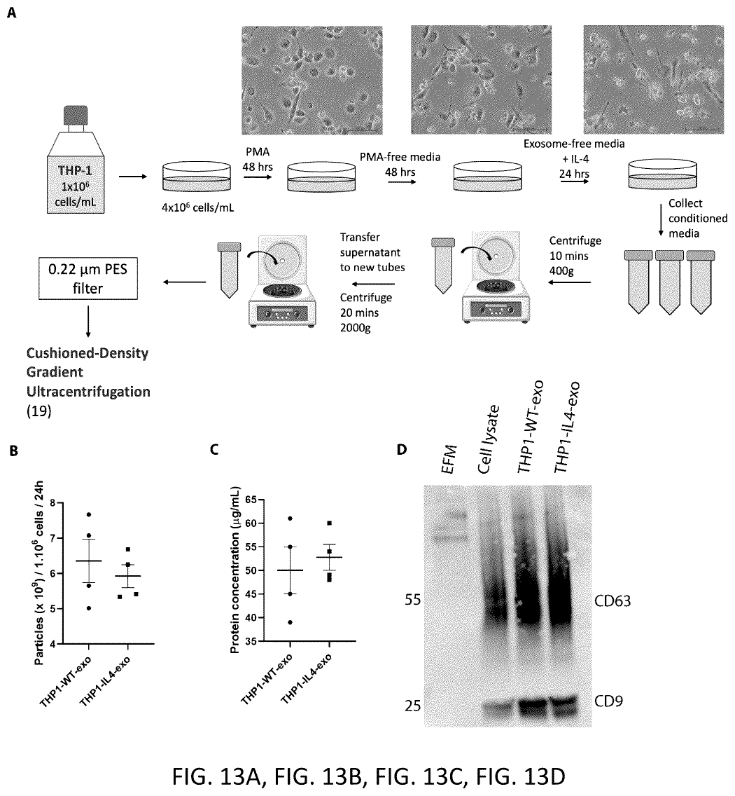

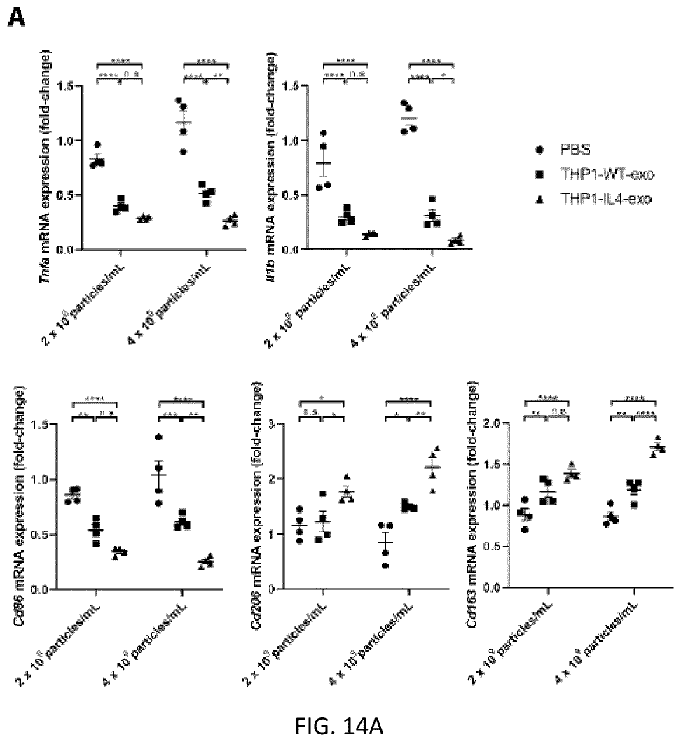

The human monocytic cell line THP-1 was differentiated to macrophages and exposed to recombinant human IL-4 for 24 hours in RPMI medium depleted of exosomes according to the flow diagram (FIG. 13A). Exosomes secreted by IL-4-exposed and naïve THP-1 macrophages were purified using cushioned-density gradient ultracentrifugation (C-DGUC), a method that allows for a gentle concentration and purification of exosomes from conditioned culture medium and biofluids reported in prior studies. Nanoparticle tracking analysis revealed similar particle concentration of 4.7×10¹⁰ and 4.1×10¹⁰ particles/mL and average mode size of 100 and 90.5 nm for naïve THP-1 exosomes (THP1-WT-exo) and IL-4-stimulated THP-1 exosomes (THP1-IL4-exo), respectively (FIGS. 1A-C). The data show that naïve or IL-4-stimulated THP-1 macrophages generally secreted the same quantity of exosomes in a 24 hr period, averaging 6×10⁹ secreted particles per million cells for both conditions (FIG. 13B). Morphological assessment of THP1-WT-exo and THP1-IL4-exo using transmission electron microscopy revealed an expected cup-shaped morphology and size averaging 100 nm (FIG. 1D). Such isolates also showed similar average protein concentrations of 50 and 53 μg/mL for THP1-WT-exo and THP1-IL4-exo, respectively (FIG. 13C). Western blot analysis showed the presence of exosomal proteins, including CD9, CD63, and CD81, and the absence of cell-associated proteins Calnexin and GM130 in a quantity of 1.5×10⁹ particles (FIG. 1E) or 1.5 ug of proteins of both THP1-IL4-exo and THP1-WT-exo (FIG. 13D).

ii. THP1-IL4-Exo Induce Immune and Metabolic Reprogramming in Recipient Macrophages

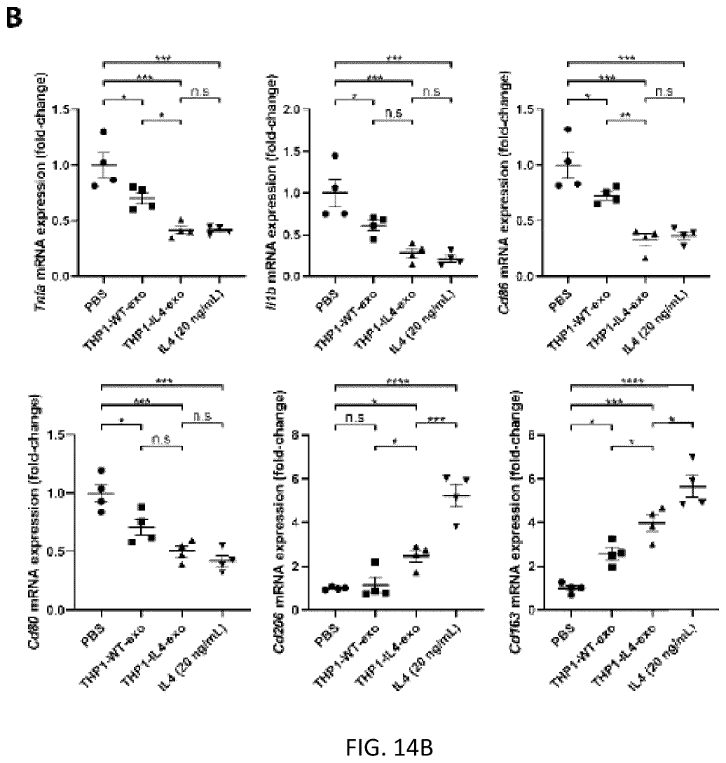

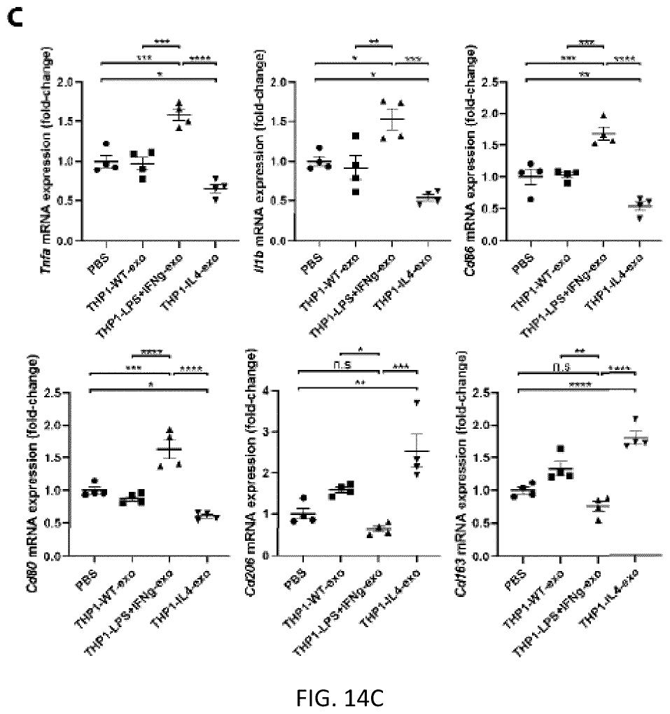

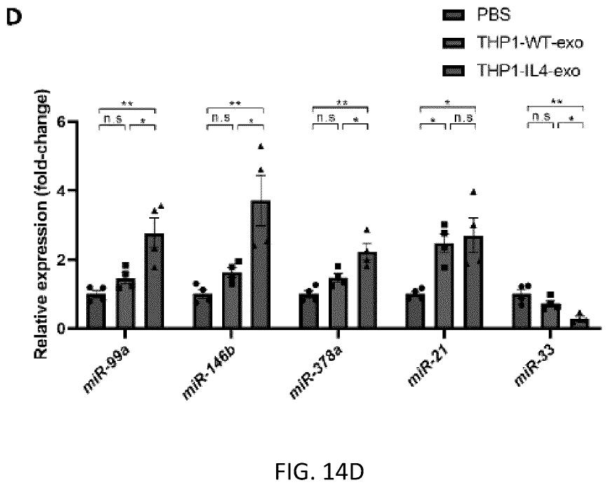

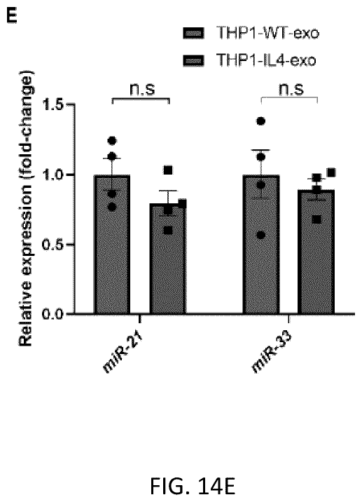

To assess in vitro signaling properties of THP-1 macrophage exosomes, naive THP-1 macrophages were treated with increasing concentrations of THP1-IL4-exo (2×10⁹ or 4×10⁹ particles/mL) or PBS for 24 hours and noted a dose response when measuring their impact on gene expression levels using qRT-PCR analysis. While recipient cells treated with 2×10⁹ particles/mL of THP1-IL4-exo displayed a downregulation in M1-associated genes (Tnf, Il1b, Cd86, and Cd80) and upregulation in M2-associated genes (Cd206 and Cd163), a treatment using 4×10⁹ particles/mL caused more profound effects (FIG. 14A). Furthermore, the control of gene expression exerted by 4×10⁹ particles/mL of THP1-IL4-exo in naïve THP-1 macrophages was similar to what was observed when treating the cells directly with 20 ng/ml of IL-4 cytokine for 24 hours (FIG. 14B). Interestingly, while THP1-WT-exo also displayed an ability to suppress the expression of pro-inflammatory markers in recipient THP-1 macrophages, their capacity to do so was two- to three-fold less potent than what was observed in cells treated with THP1-IL4-exo or directly with IL-4 (FIGS. 14A-B).

In line with prior findings with IL-4-stimulated BMDM exosomes, treatment with THP1-IL4-exo also upregulated the expression of anti-inflammatory M2 markers (Cd206 and Cd163) which was not observed with THP1-WT-exo or PBS treatment, although the effect was two-fold less when compared to a direct treatment with IL-4 at a concentration of 20 ng/ml (FIG. 14A-B). Interestingly, it was also found that exosomes derived from THP-1 macrophages treated with 100 ng/mL LPS and 20 ng/mL IFN-γ for 24 hours upregulated the expression of pro-inflammatory cytokines (Tnf and Il1b) and M1 markers (Cd86 and Cd80) (FIG. 14C). These results demonstrate the capacity for THP-1 macrophage exosomes to communicate pro- and anti-inflammatory signals depending on the polarization state of their parental cells.

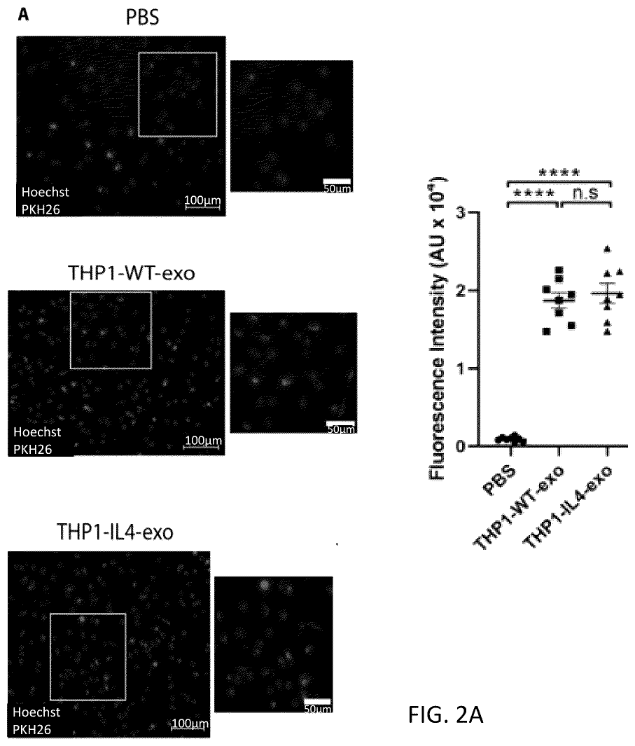

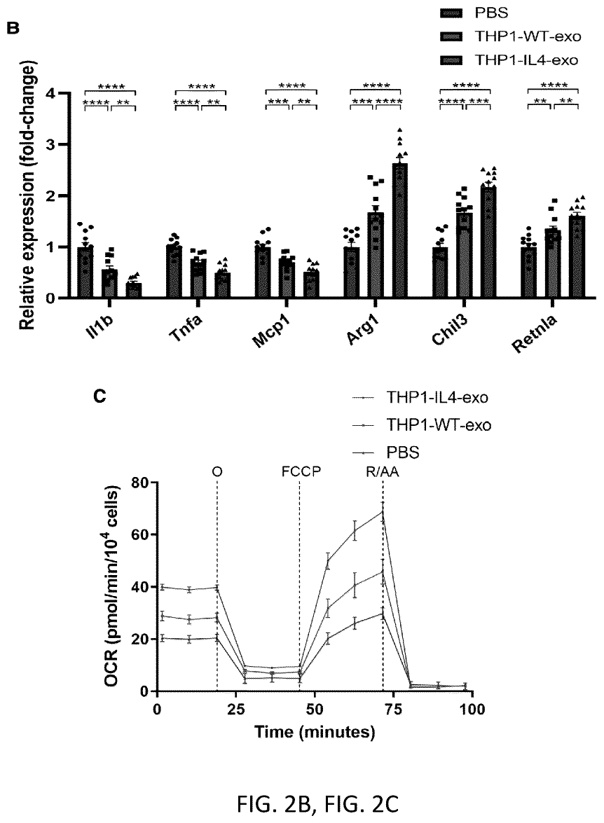

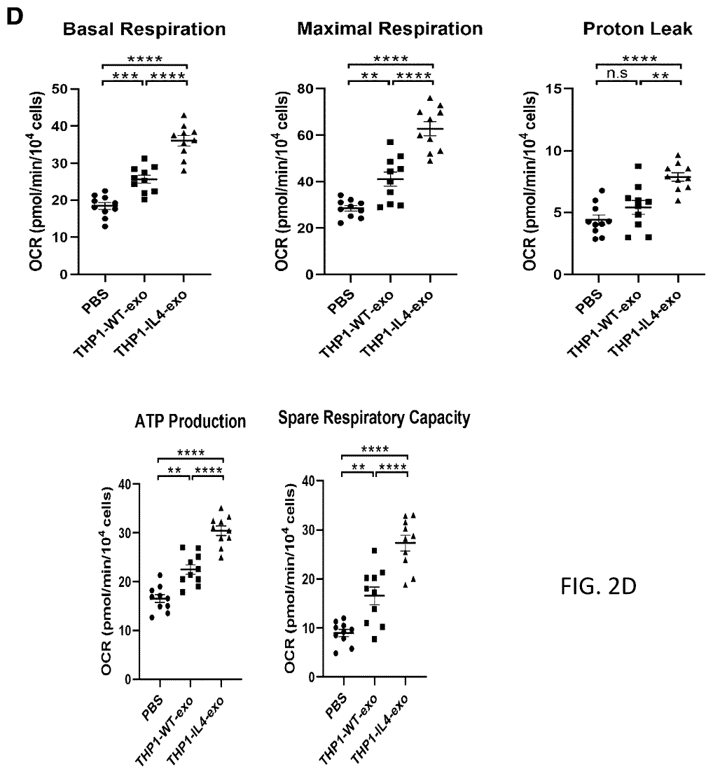

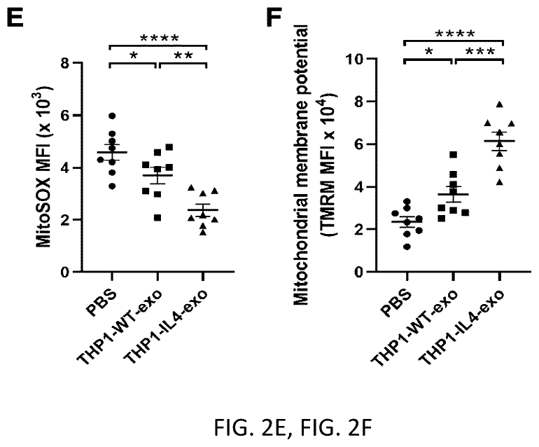

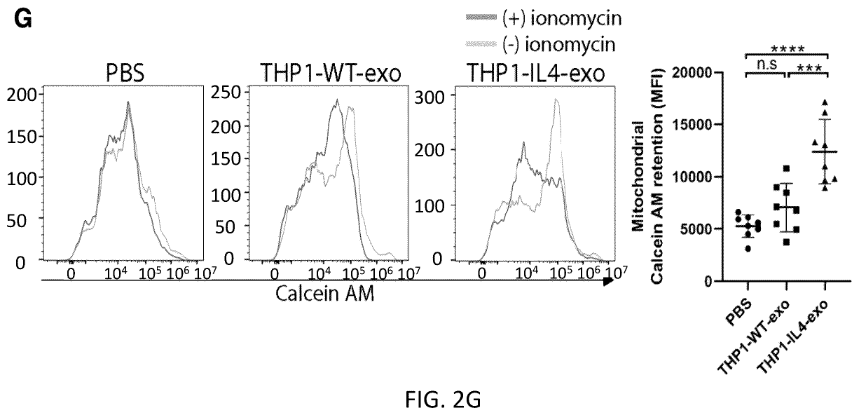

In testing their capacity to control metabolic pathways as a source of their anti-inflammatory signaling, THP1-IL4-exo was exposed to cultured primary BMDM. Fluorescence intensity detection of PKH26-labeled THP-1 exosomes showed similar internalization efficiencies by recipient BMDMs (FIG. 2A) and capacity to drive anti-inflammatory gene expression (FIG. 2B) as observed in recipient THP-1 macrophages (FIGS. 14A-C). Next, the capacity of THP1-IL4-exo to drive metabolic reprogramming required to fuel macrophage polarization and their effector functions was tested. The oxygen consumption rate (OCR) was measured using the Seahorse Mito Stress Assay. The data show that BMDM treated with THP1-IL4-exo displayed enhanced oxidative phosphorylation (OXPHOS) as seen by elevated basal and maximal respiration associated with a higher proton leak and ATP production compared with BMDM treated with PBS or THP1-WT-exo (FIGS. 2C, D). To further document their metabolic reprogramming properties, mitochondrial activity was measured in BMDM treated with THP1-IL4-exo, THP1-WT-exo, or PBS alone for 24 hours. The findings revealed a reduced accumulation of superoxide in mitochondria as detected by flow cytometric analysis of BMDM stained with MitoSOX, with more potent effects observed in THP1-IL4-exo treatments (FIG. 2E). Next, measurements of mitochondrial membrane potentials (AY′m) using flow cytometric analysis of tetramethylrhodamine (TMRM) staining further support improved mitochondrial activity in BMDM that had been treated with THP1-WT-exo and THP1-IL4-exo, with more profound effects observed when exposing cells to THP1-IL4-exo (FIG. 2F). Furthermore, THP1-IL4-exo were found to potently control mitochondrial permeability by modulating the dynamic opening of the transition pores in BMDM, resulting in improved mitochondrial Calcein AM retention as detected by flow cytometry (FIG. 2G). Together, the data demonstrate the ability for THP1-IL4-exo to confer robust immune and metabolic reprogramming properties in recipient macrophages.

iii. THP1-IL4-Exo Induce Lipophagy and Modulate microRNA Levels that Control Metabolism in Recipient Macrophages

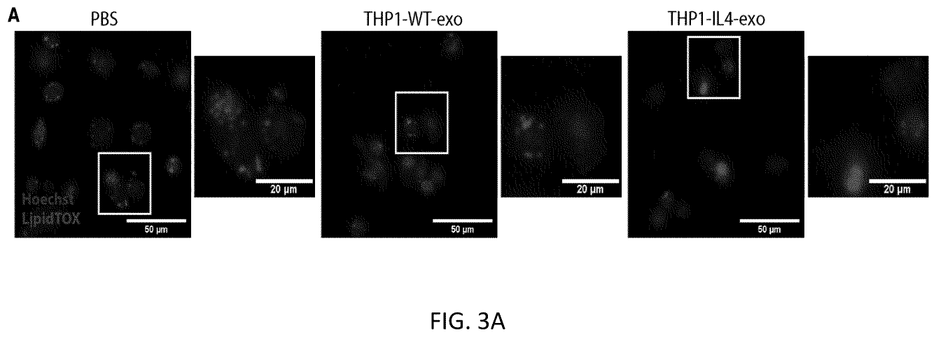

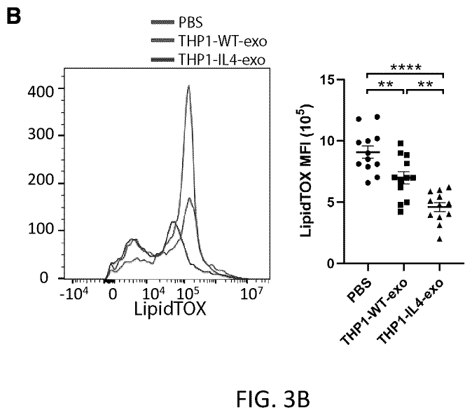

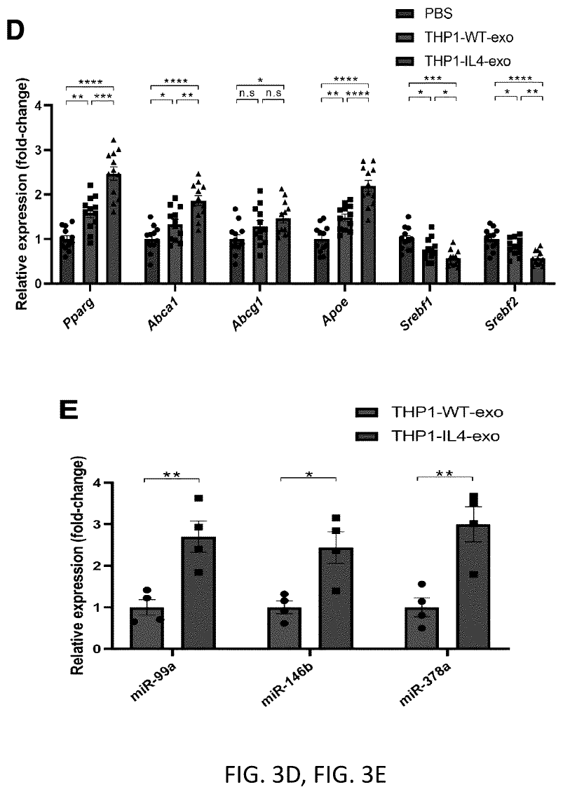

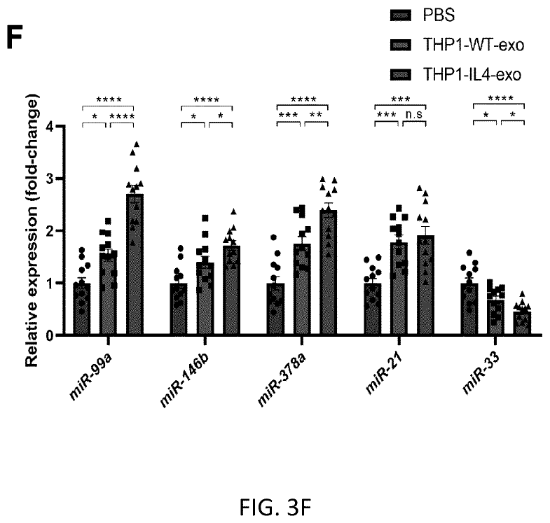

To further examine the extent to which THP1-IL4-exo reprogram cellular metabolism, their impact on lipid droplet sizes and densities were assessed in recipient BMDM stained with LipidTOX using fluorescent microscopy and flow cytometry. The findings show that THP1-IL4-exo treatments reduced neutral lipid accumulation in BMDM (FIGS. 3A, B). Furthermore, gene expression analysis of RNA extracted from these cells revealed substantial increases in the expression of genes associated with lipophagy that included Ulk1, Pnpla2, Lipe, Map1lc3a, and Map1lc3b (FIG. 3C). A robust increase in the expression of peroxisome proliferator activated receptor gamma (PPARγ), Pparg, was also found along with its target genes involved in cholesterol efflux that included Abca1 and Apoe (FIG. 3D). Remarkably, THP1-IL4-exo exerted opposite effects in controlling the expression of genes responsible for cellular lipid synthesis and uptake including the sterol regulatory element-binding protein 1 and 2 (SREBP-1 and SREBP-2), Srebf1 and Srebf2 (FIG. 3D).

M1 and M2-polarized primary bone marrow derived macrophages release select sets of microRNAs into exosomes that can communicate inflammatory and metabolic signaling to recipient macrophages, hematopoietic stem/progenitor cells (HSPC) and adipocytes. In testing whether THP-1 macrophages can release similar sets of microRNAs as those reported to be highly enriched in IL-4-treated BMDM exosomes, levels of microRNA-99a/146b/378a were measured. The findings show an enrichment of all three of these microRNAs in THP1-IL4-exo as compared to THP1-WT-exo (FIG. 3E). Interestingly, naïve THP-1 macrophages and murine BMDM treated with THP1-IL4-exo for 24 hours also displayed increased levels of these microRNAs as compared to cells treated with either THP1-WT-exo or PBS (FIGS. 14D and 3F), supporting their communication from exosomes. Stemming from their potent capacity to polarize macrophages and control their lipid metabolism, the impact of THP1-IL4-exo in modulating cellular levels of miR-21-5p, a central regulator of macrophage polarization, along with miR-33-5p that is recognized as a regulatory hub for energy metabolism were tested in macrophages. While increased expression of miR-21-5p was noted in THP-1 macrophages and BMDM treated with THP1-IL4-exo, opposite effects were uncovered when looking at miR-33-5p. Indeed, a three-fold reduction in levels of miR-33-5p was noted in cells treated with THP1-IL4-exo (FIGS. 14D and 3F). This finding is in line with the data documenting substantial reduction in Srebf2 mRNA (FIG. 3D) that serves as its host. No significant difference in miR-21-5p and miR-33-5p cargo were noted in THP1-IL4-exo as compared to THP1-WT-exo (FIG. 14E). Taken together, these results support a capacity for THP1-IL4-exo to communicate anti-inflammatory and metabolic control in macrophages by modulating levels of cellular microRNA recognized to play critical roles in regulating lipophagy, energy metabolism and inflammatory signaling in these cells.

iv. THP1-IL4-Exo Promote Beiging in Recipient 3T3-L1 Adipocytes by Modulating Cellular microRNA

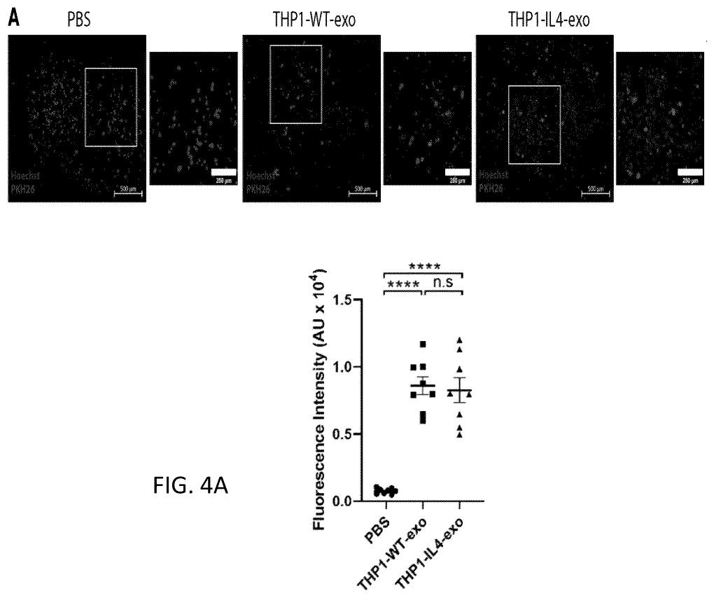

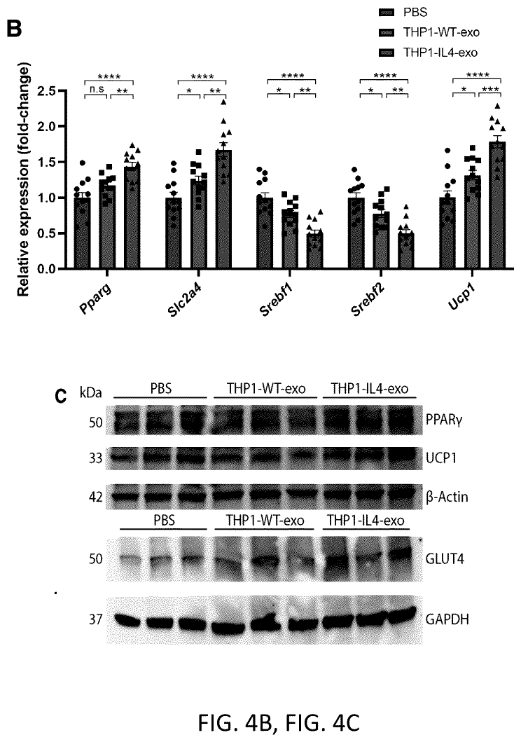

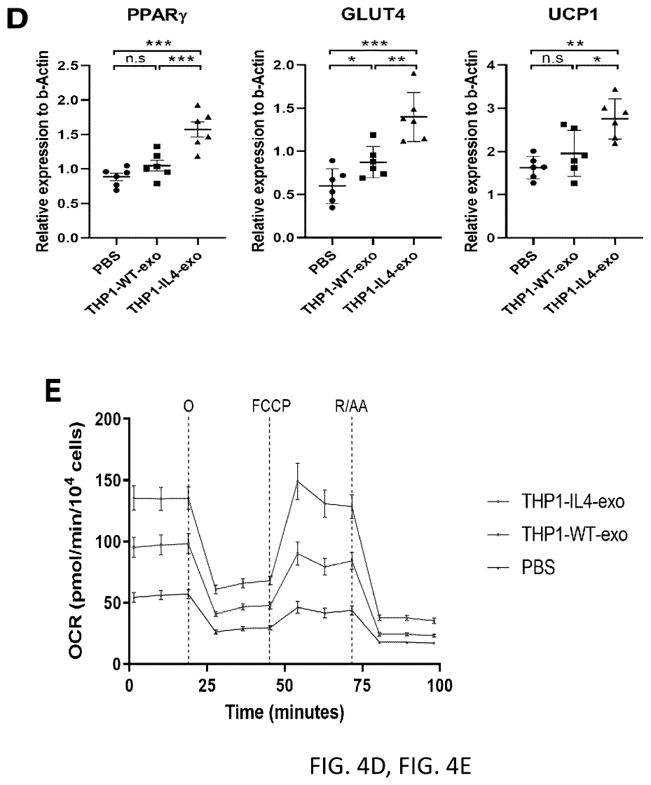

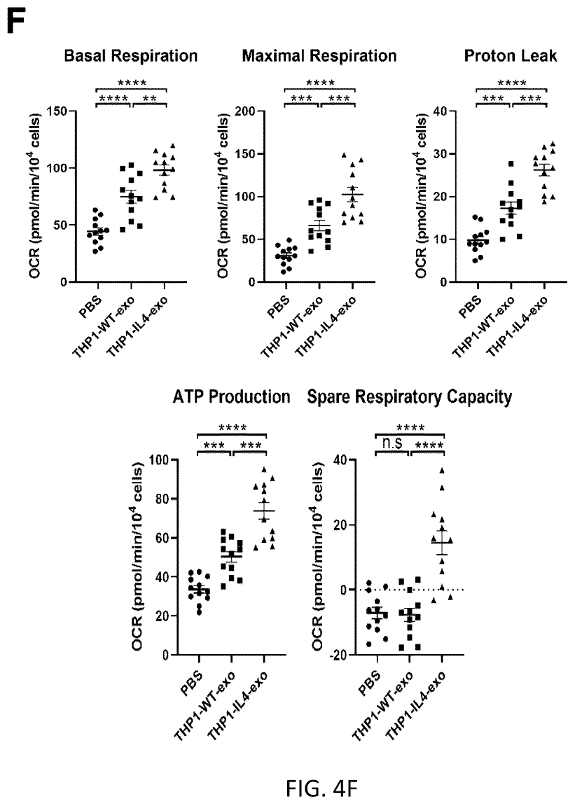

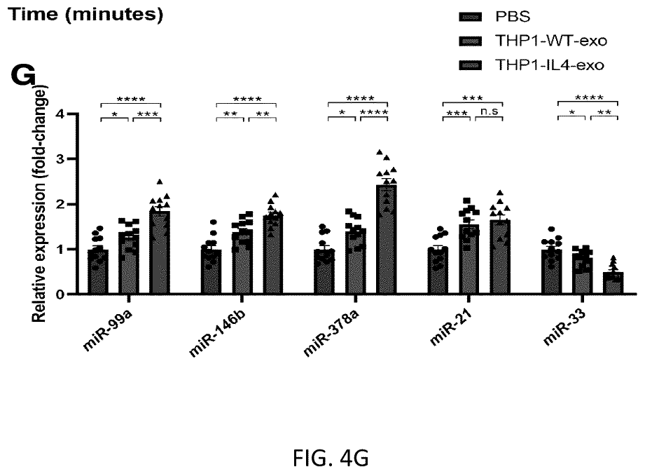

While studies have shown that exosomes produced by primary mouse macrophages can control insulin sensitivity and metabolism in mouse adipocyte, whether those produced by human THP-1 cells can exert similar properties is not known. To test this possibility, fully differentiated 3T3-L1 adipocytes were treated with THP1-IL4-exo or control for 24 hours. Fluorescence intensity detection of PKH26-labeled exosomes produced by THP-1 cells exposed to IL-4 or control showed similar internalization efficiencies in recipient 3T3-L1 adipocytes (FIG. 4A). THP1-IL4-exo treatments increased expression in Pparg and its target gene Slc2a4 (GLUT4). 28, as well as Ucp1 (FIG. 4B). A concomitant reduction was also noted in Srebf1 and Srebf2 (FIG. 4B). Western blot analysis confirmed increased protein levels for the gene products of PPARγ, GLUT4, and UCP1 in cell lysates (FIG. 4C-D). Having observed pro-thermogenic properties conferred by THP-1 macrophage exosomes upon recipient adipocytes, it was next wondered whether they could also enhance mitochondrial respiration in these cells by testing the OCR using the Seahorse Mito-Stress Assay. Data shown in FIG. 4E-F demonstrate the capacity for THP1-IL4-exo to profoundly increase OXPHOS activity in 3T3-L1 adipocytes as compared to treatments with THP1-WT-exo or PBS alone.

To further assess mechanism through which THP1-IL4-exo induce metabolic activity in 3T3-L1 adipocytes, the expression levels of microRNAs were detected and found to be altered in BMDM and THP-1 cells treated with THP-1 exosomes (FIGS. 14D and 3F). In doing so, an enrichment of miR-99a-5p, miR-146b-5p, and miR-378a-3p was detected in 3T3-L1 adipocytes treated with THP1-IL4-exo as compared to those treated with either THP1-WT-exo or PBS (FIG. 4G). Next the expression of microRNAs central to adipocyte beiging and homeostasis was measured including miR-21-5p and miR-33-5p and it was found to show opposing sensitivity to THP1-IL4-exo in cultured macrophages (FIGS. 14D and 3F). While it was noted a nearly two-fold increase in miR-21-5p levels (FIG. 4G), a sharp two-fold reduction in miR-33-5p levels was detected in 3T3-L1 adipocytes treated with THP1-IL4-exo (FIG. 4G). Together, these findings provide compelling evidence supporting THP1-IL4-exo as a source of beiging and OXPHOS in recipient adipocytes by modulating microRNA that parallel those seen in recipient BMDM.

v. THP1-IL4-Exo Induce Lipophagy and Mitochondrial Activity During the Differentiation of 3T3-L1 Adipocytes.

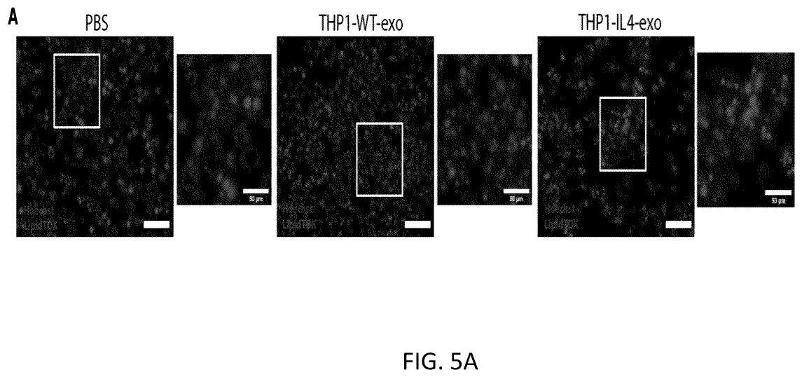

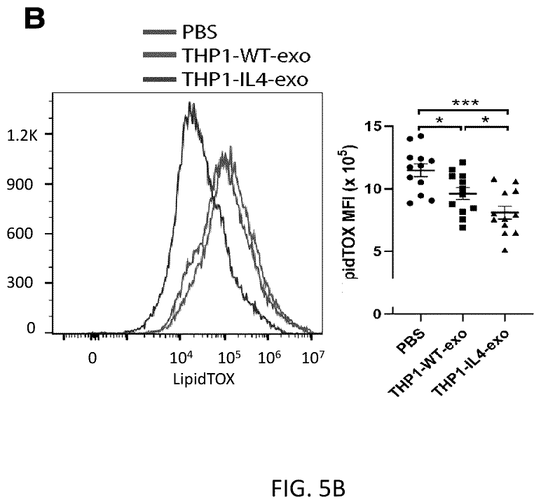

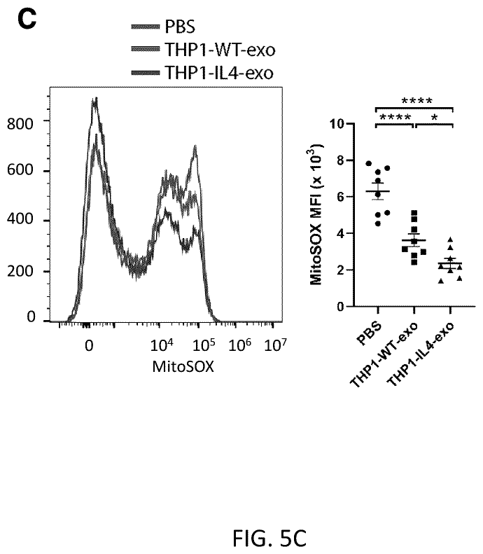

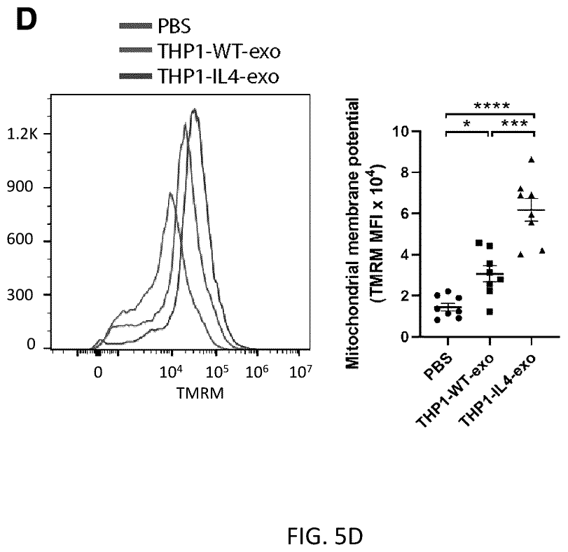

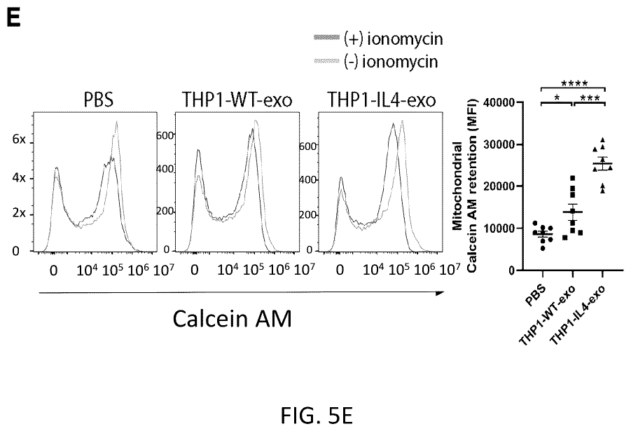

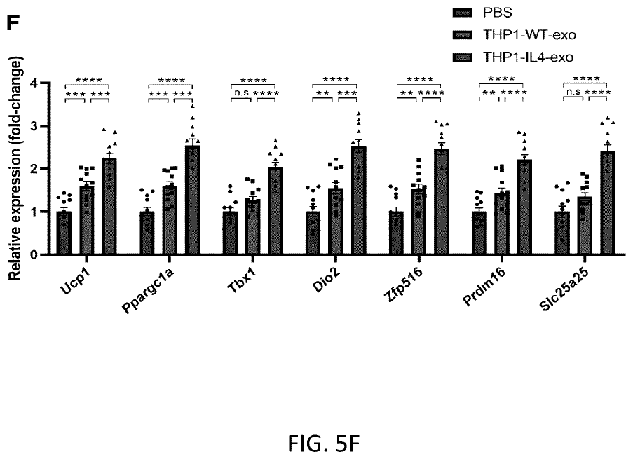

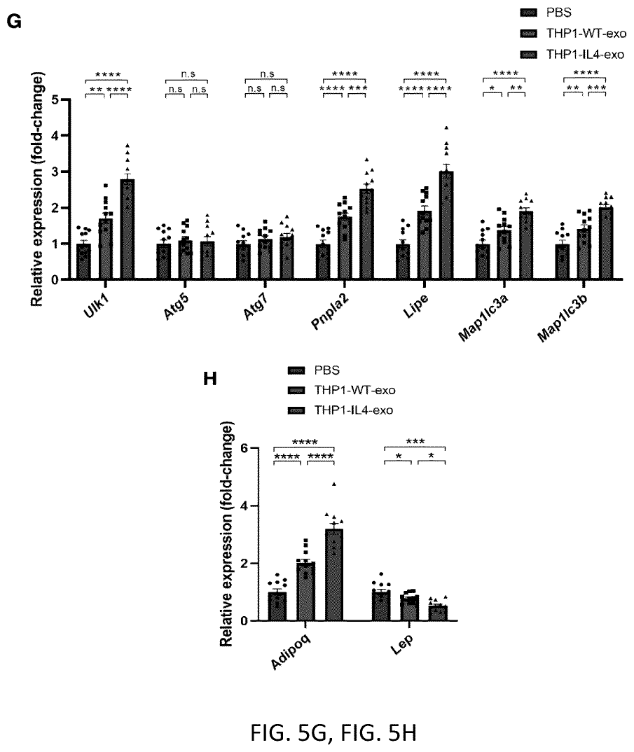

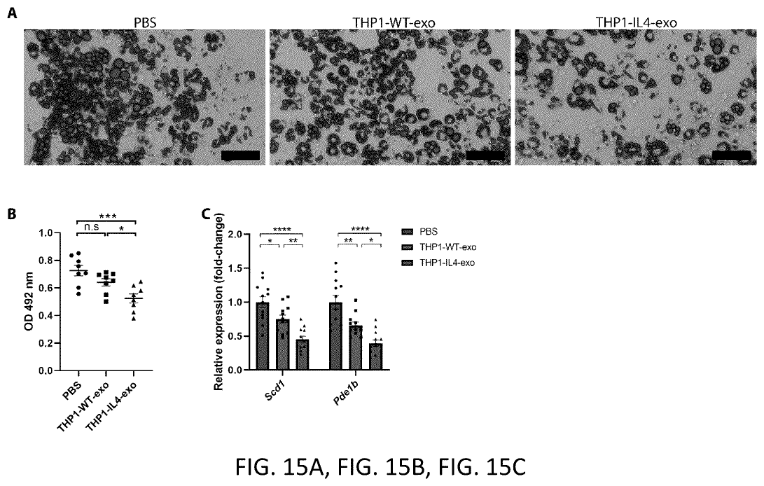

Metabolic activities controlled by THP1-IL4-exo were examined during 3T3-L1 preadipocyte differentiation performed in basic DMEM medium with 1 μg/ml bovine insulin, 0.5 mM methylisobutylxanthine (IBMX), and 1.0 mM dexamethasone (DEX) for two days as widely reported. Cells were treated with THP1-IL4-exo every two days at a concentration of 4×10⁹ particles/ml along with a fresh change of DMEM medium supplemented with 1 μg/ml bovine insulin until fully differentiated on day 15. Monitoring cellular neutral lipids using LipidTOX staining, revealed that THP1-IL4-exo treatments reduced neutral lipid droplet accumulation during 3T3-L1 preadipocytes' differentiation process. In contrast to adipocytes treated with PBS that displayed large lipid vacuoles, those treated repeatedly with THP1-exo displayed far fewer and smaller lipid vacuoles that were even further reduced in adipocytes treated with THP1-IL4-exo (FIG. 5A). Quantification of neutral lipid staining by flow cytometric analysis confirmed the microscopic observations. Cells exposed to THP1-IL4-exo displayed lower mean fluorescent intensity of LipidTOX as compared to cells treated with either THP1-WT-exo or PBS alone (FIG. 5B). Quantifying Oil Red O extracted from sets of cells treated in a similar manner further confirmed benefits that THP1-IL4-exo exerted to reduce neutral lipid accumulation in differentiated 3T3-L1 adipocytes (FIGS. 15A-B).

To further document evidence of beiging in 3T3-L1 cells exposed to THP-1 macrophage exosomes, flow cytometric analyses was used to monitor three established parameters of mitochondrial activity. In detecting levels of MitoSOX dye fluorescence, a marked reduction of mitochondrial superoxide was noted in 3T3-L1 adipocytes treated with THP1-IL4-exo that was less prominent in those treated with THP1-WT-exo (FIG. 5C). Next, measurements of TMRM levels revealed increased mitochondrial membrane potential Δψm in 3T3-L1 adipocytes repeatedly treated with THP-1 exosomes, with more profound effects observed when using THP1-IL4-exo treatments (FIG. 5D). Finally, increased mitochondrial Calcein AM retention was observed in THP1-IL4-exo-treated 3T3-L1 adipocytes (FIG. 5E), demonstrating a similar level of control over mitochondrial permeability as observed in BMDM (FIG. 2G). Together, these data demonstrate the capacity for THP1-IL4-exo signaling to improve mitochondrial health and functions in recipient 3T3-L1 adipocyte during differentiation, a hallmark of beiging in white adipocytes.

In assessing pathways associated with beiging in 3T3-L1 adipocytes treated with THP1-IL4-exo, the expression levels of genes associated with thermogenesis was measured. The findings revealed a concerted upregulation of canonical markers of adipose tissue beiging that included Ucp1, Pparge1a, Tbx1, Dio2, Zfp516, Prdm16, and Slc25a25. Importantly, their level of upregulation was increased when adipocytes were treated with THP1-IL4-exo rather than THP1-WT-exo (FIG. 5F). In agreement with the findings seen in recipient macrophages, such treatments also led to an upregulated expression of genes associated with lipophagy during adipocyte beiging, namely Ulk1, Ppla2, Lipe, Map1lc3a, and Map1lc3b (FIG. 5G). As with other tested parameters, the modulation of these genes was more potent in adipocytes treated with THP1-IL4-exo. Lastly, an increased expression of adiponectin (Adipoq) was documented with a concomitant decrease in the expression of Leptin (Lep) (FIG. 5H). Taken together, these findings reveal that THP1-IL4-exo can serve to drive beiging during the differentiation of 3T3-L1 preadipocyte into adipocytes by inducing lipophagy and improving mitochondrial activity that together increase the expression of adipokines central to the regulation of obesity and diabetes.

vi. THP1-IL4-Exo Resolve Cardiometabolic Inflammation in Mice with Diet-Induced Hyperlipidemia, Obesity, and Insulin Resistance

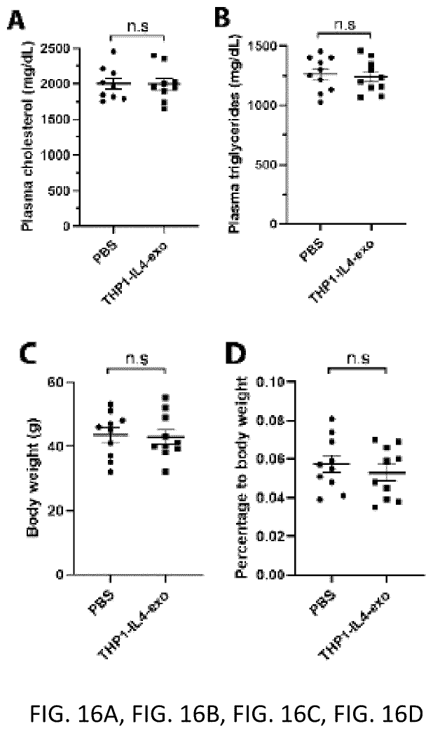

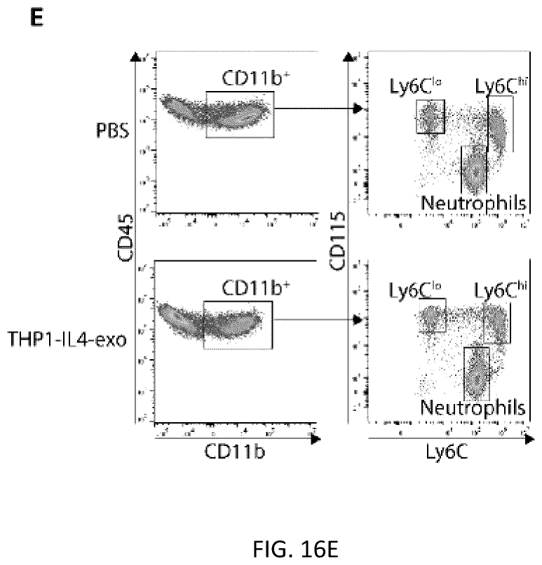

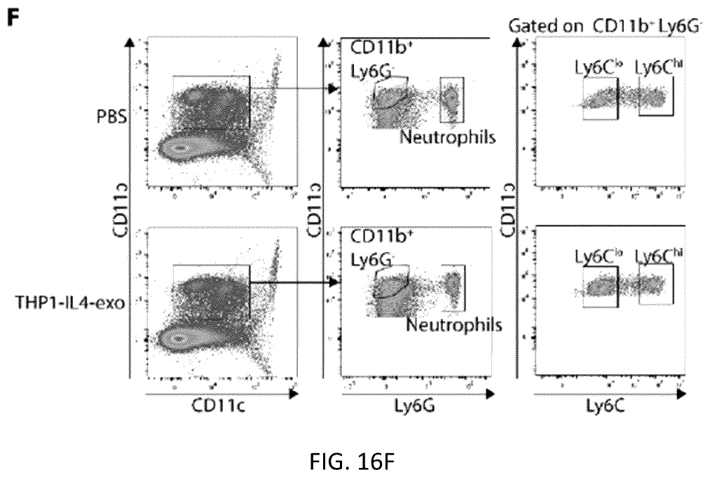

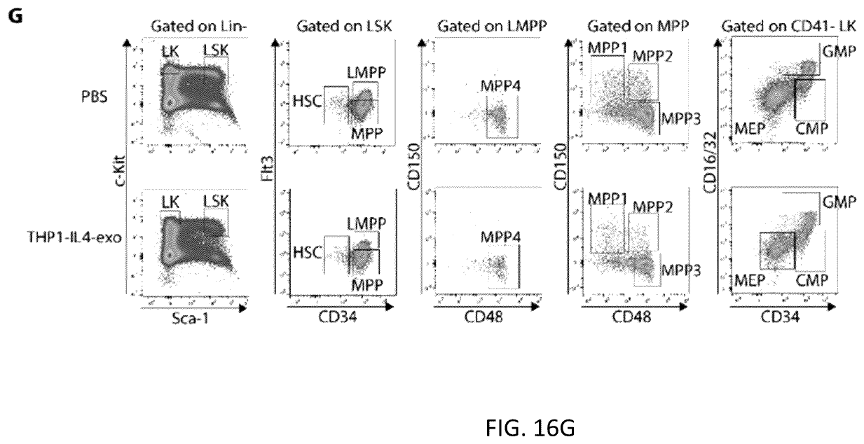

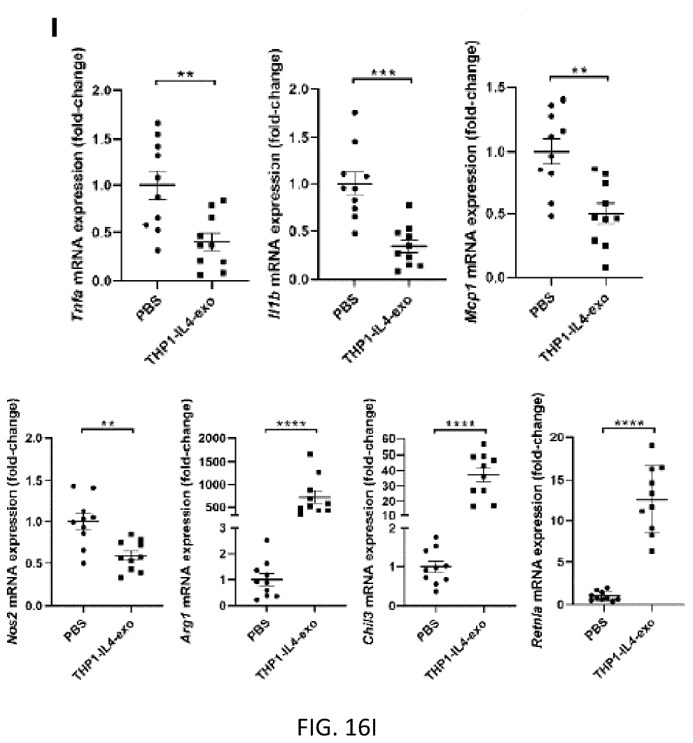

In light of provocative evidence pointing to THP1-IL4-exo as potent mediators of cellular metabolic reprogramming in cultured macrophages and adipocytes, their capacity to control cardiometabolic tissue inflammation was explored in mice. This was done by repeatedly treating obese mice with intraperitoneal (i.p.) infusions of THP-1 macrophage exosomes and monitored parameters of inflammation as recently reported in the studies of Apoe-mice treated with exosomes derived from IL-4-exposed BMDM. To avoid complications associated with a loss of apoE expression on adiposity and inflammation, hypomorphic Apoe^{h/h} Ldlr^{-/-} mice were studied that were previously reported as a model of spontaneous hyperlipidemia and atherosclerosis in the setting of reduced cellular Apoe expression that results in plasma apoE- and apoB-lipoprotein accumulation due to impaired clearance mechanisms in the liver. For the purpose of this study, 20-week-old Apoe^{h/h} Ldlr^{-/-} mice had been fed a diet rich in saturated fat and sucrose (Western diet) for 10 weeks to induce obesity and insulin resistance. Owing to reduced Apoe expression levels in all tissues, Apoe^{h/h} Ldlr^{-/-} mice were more sensitive to Western diet feeding than Ldlr^{-/-} mice, and therefore displayed greater plasma cholesterol and triglyceride levels averaging 2000 mg/dL and 1300 mg/dL, respectively (FIGS. 16A-B).

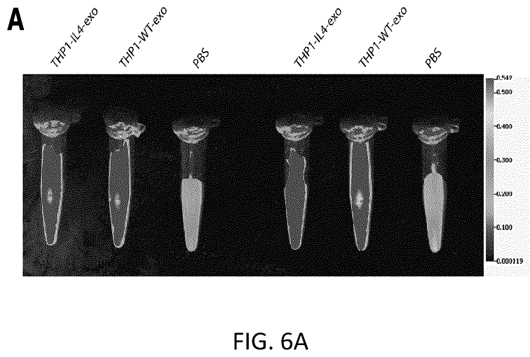

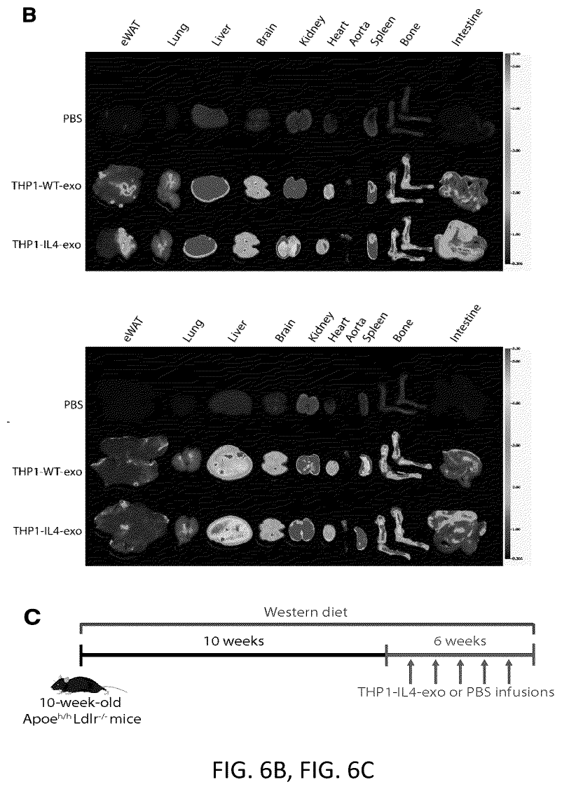

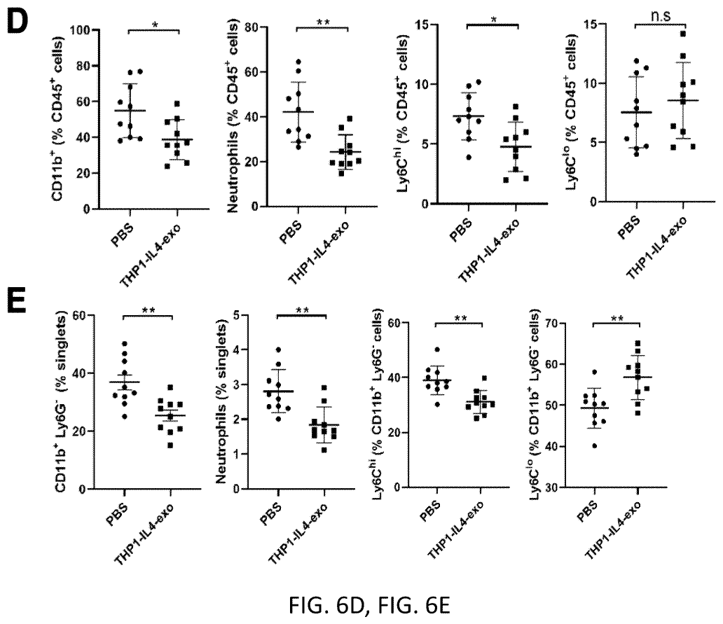

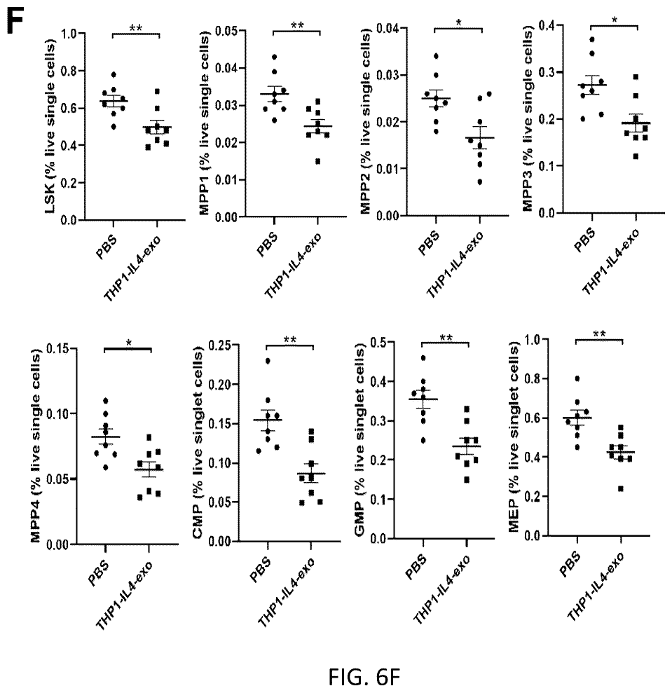

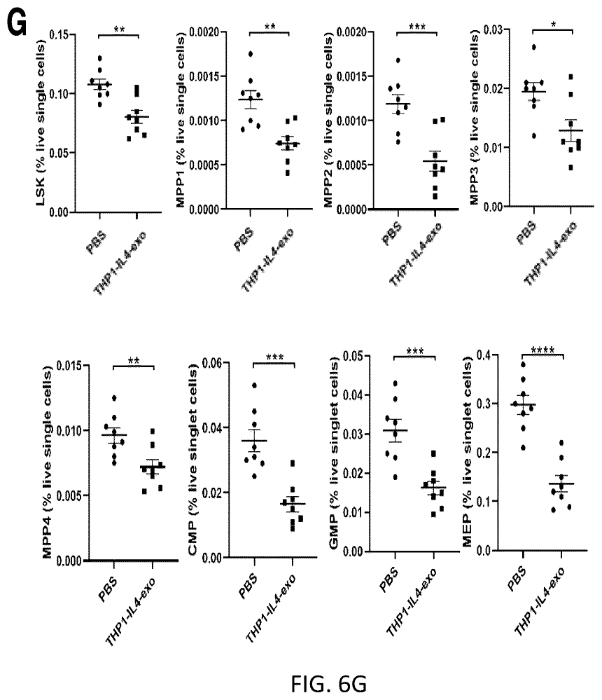

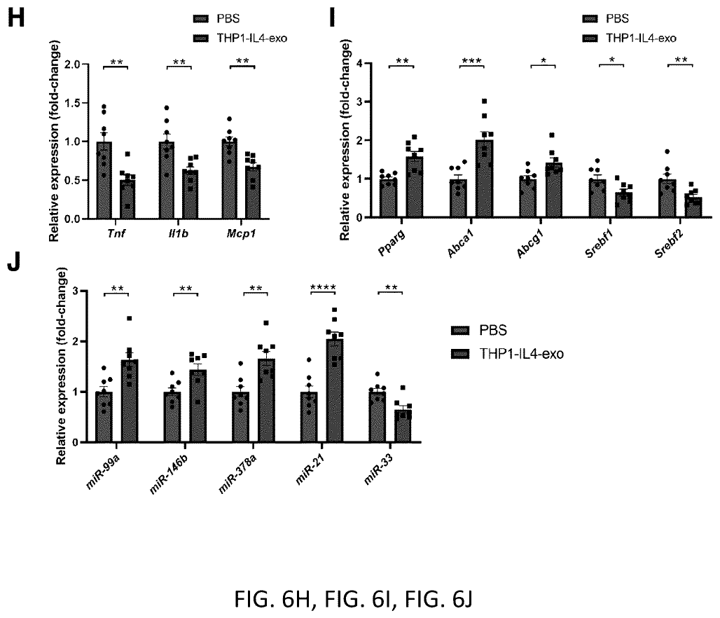

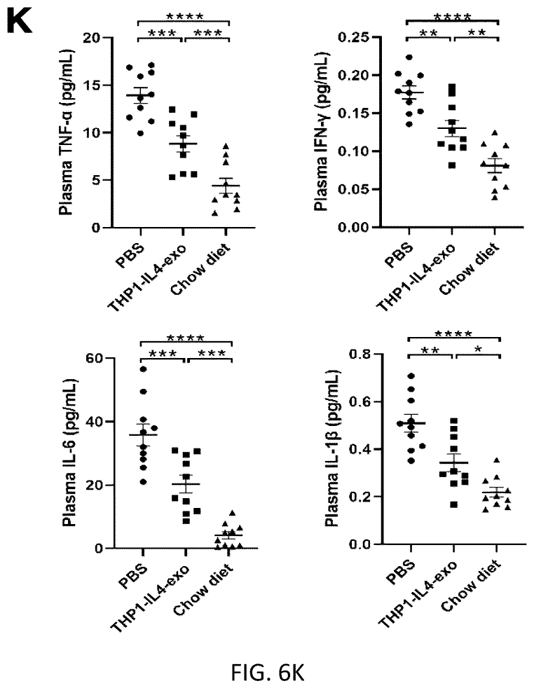

To assess their biodistribution, THP-1 macrophage exosomes were labelled with DiR (DiIC18 (7) (1,1′-Dioctadecyl-3,3,3′,3′ Tetramethylindotricarbocyanine Iodide) and infused i.p. into 20-week-old Apoe^{h/h} Ldlr^{-/-} mice that had been fed the Western Diet for 10 weeks. Six hours post infusion, the presence of DiR-positive exosomes was detected in the circulation (FIG. 6A), as well as the epididymal white adipose tissue (eWAT), lung, liver, brain, kidney, aorta, spleen, bone, and intestine (FIG. 6B). Data shown in FIG. 6B also demonstrate a similar pattern of biodistribution between THP1-WT-exo and THP1-IL4-exo that included the eWAT, liver, bone marrow, and spleen.

Based on the similar pattern of biodistribution displayed by the two forms of THP-1 macrophage exosome preparations, the in vivo properties of THP1-IL4-exo were studied due to their more potent capacity to control inflammation and metabolic activities observed through the in vitro studies in FIGS. 2-5. Cohorts of 20-week-old Western diet-fed Apoe^{h/h} Ldlr^{-/-} mice were treated with either 1×10¹⁰ particles of THP1-IL4-exo, a dose that was determined to represent 2 to 5% of total exosomes in mouse plasma, or PBS 3 times/week (every 2 days) for 6 weeks while the mice were kept on Western diet. At the end of the exosome infusion period, all mice displayed similar obesity with an average weight of 42 grams for both THP1-IL4-exo-infused and PBS-infused group (FIG. 16C). Moreover, all mice displayed a similar eWAT fat-to-body ratio of 0.05 (FIG. 16D). A diagram of experimental parameters is shown in FIG. 6C. In agreement with recent studies of BMDM exosomes, the infusion of THP1-IL4-exo into mice provoked changes in hematopoiesis and leukocyte numbers in the circulation. Indeed, mice infused with THP1-IL4-exo displayed a profound reduction in circulating CD45⁺ CD11b⁺ myeloid cells (FIGS. 16E and 6D). Significantly less inflammatory Ly6C^{hi} monocytes and neutrophils were also found in the circulation of mice infused with THP1-IL4-exo (FIGS. 16E and 6D). In addition, a reduced accumulation of splenic CD11b⁺ Ly6G-monocytes and CD11b⁺ Ly6G⁺ neutrophils were observed in mice infused with THP1-IL4-exo (FIGS. 16F and 6E). THP1-IL4-exo treated mice also displayed reduced numbers of Ly6C^{hi} and increased Ly6C^{lo} subpopulations of splenic CD11b⁺ Ly6G⁻ monocytes (FIGS. 16F and 6E). The reduced numbers in circulating and splenic myeloid cells were corroborated by a marked reduction in hematopoietic stem/progenitor cells (HSPCs) in the bone marrow and spleen. This included reduced numbers of Lin⁻ Sca-1⁺ c-Kit⁺ (LSK) cells, multipotent progenitor cells (MPP1-4), common myeloid progenitors (CMP), granulocyte-macrophage progenitors (GMP), and megakaryocyte-erythroid progenitors (MEP), in the bone marrow and spleen (FIGS. 16G-H, 6F-G).

In seeking to uncover mechanisms through which THP1-IL4-exo controlled hematopoiesis when infused into Western diet-fed Apoe^{h/h} Ldlr^{-/-} mice, their capacity to reprogram metabolic and inflammatory gene expression in peritoneal macrophages and circulating Ly6C^{hi} monocytes isolated by Fluorescence-Activated Cell Sorting (FACS) was noted (FIGS. 161 and 6H-I). This included by decreasing the expression of pro-inflammatory cytokines Tnf, Il1b, and Mep1 (FIGS. 161 and 6H). In contrast, THP1-IL4-exo upregulated the expression of Pparg, Abca1 and Abcg1, and decreased levels of miR-33-5p and its host gene Srebf1 and Srebf2 (FIGS. 6I-J). A mechanistic basis to explain this level of cellular gene expression control likely stems from increases in levels of miR-99a-5p/146b-5p/378a-3p and miR-21-5p in Ly6C^{hi} monocytes (FIG. 6J). Finally, the control of leukocyte activation via THP1-IL4-exo also led to a profound reduction in plasma cytokines levels TNF-α, IFN-γ, IL-6, and IL-1β in mice infused with THP1-IL4-exo as determined using a multiplex immunoassay analysis (FIG. 6K). Together, these results support a role for THP1-IL4-exo in contributing to actively reprogram cellular metabolism and inflammatory function in hematopoietic compartments of mice continually maintained on a Western diet.

vii. Infusions of THP1-IL4-Exo Suppress Leukocyte Expansion in Aortic, Hepatic, and Adipose Tissues of Hyperlipidemic & Obese Mice

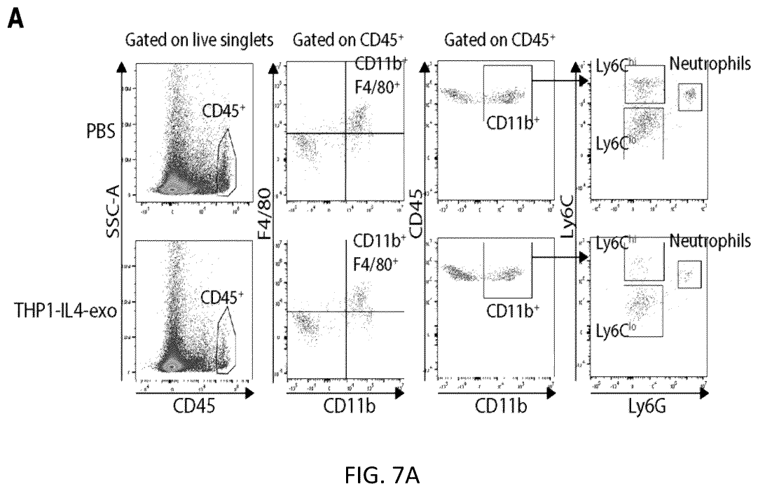

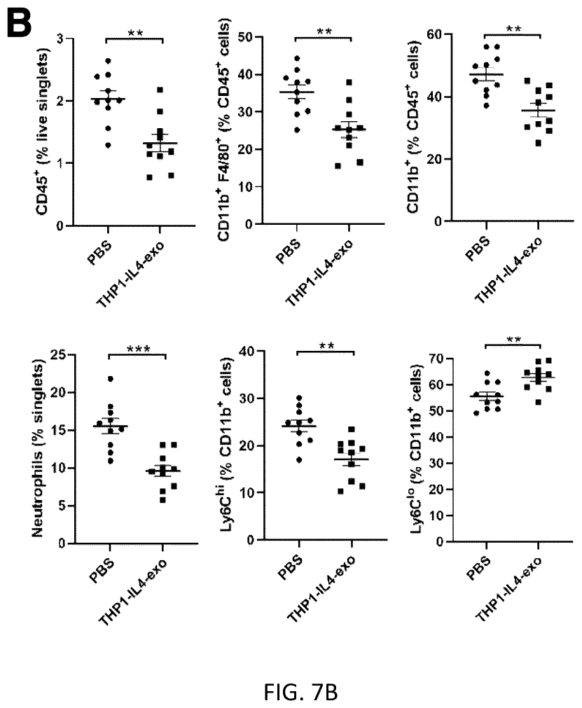

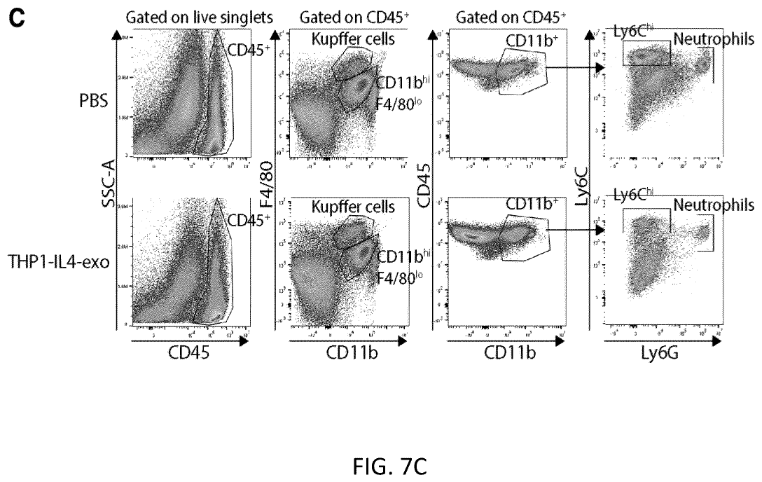

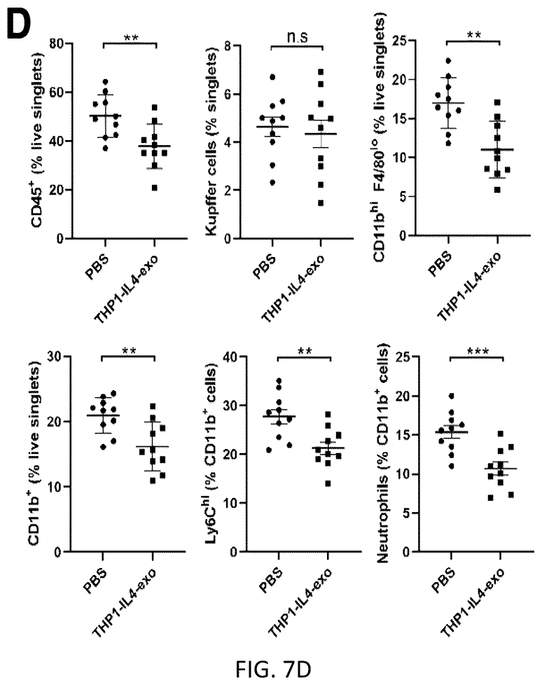

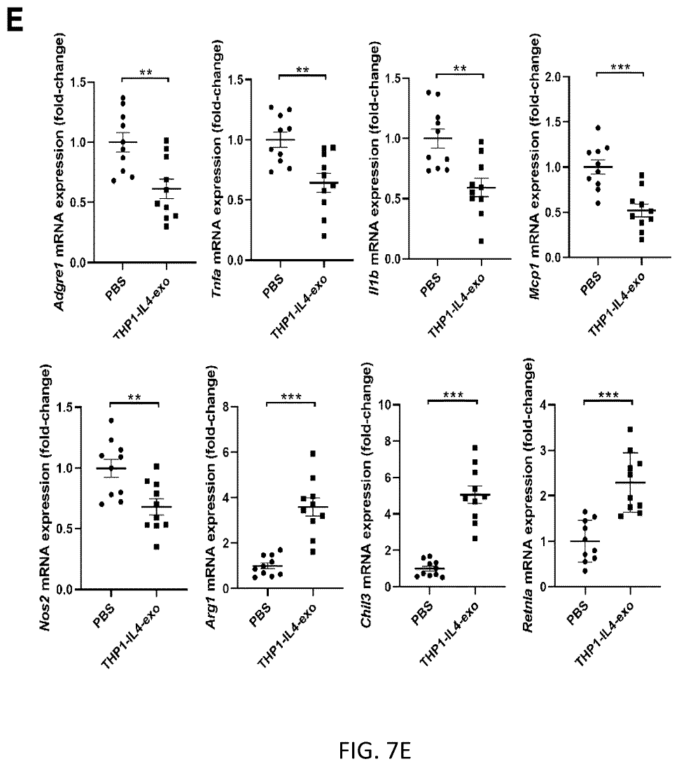

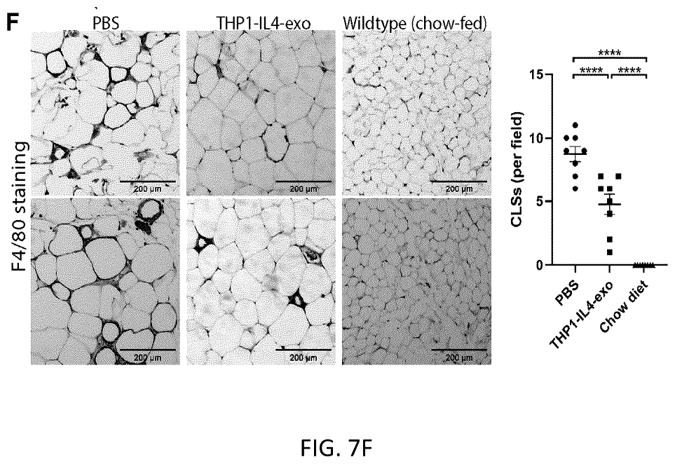

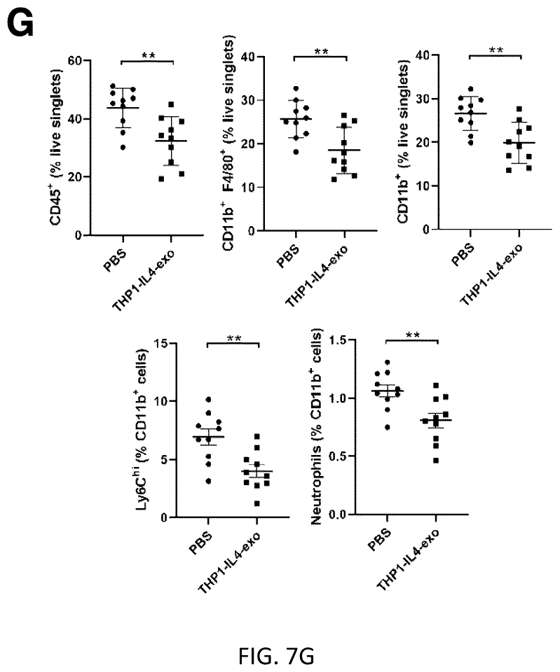

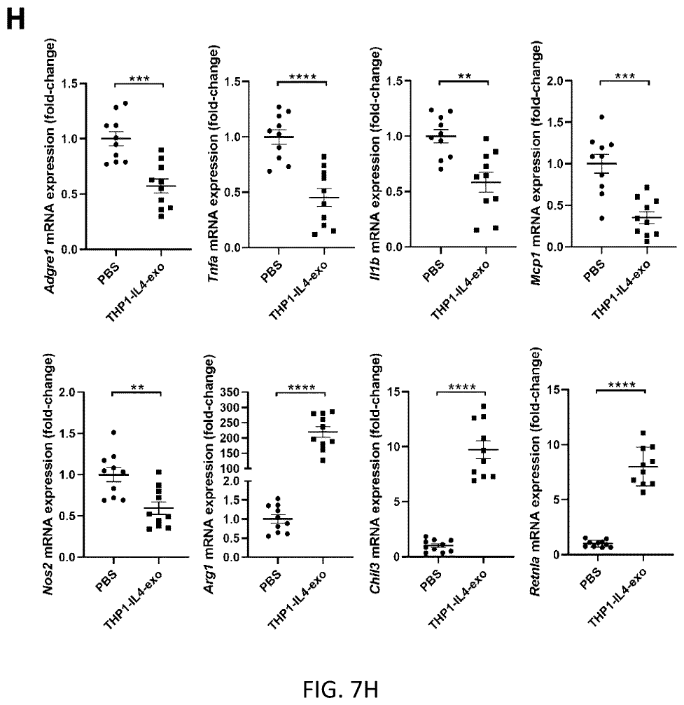

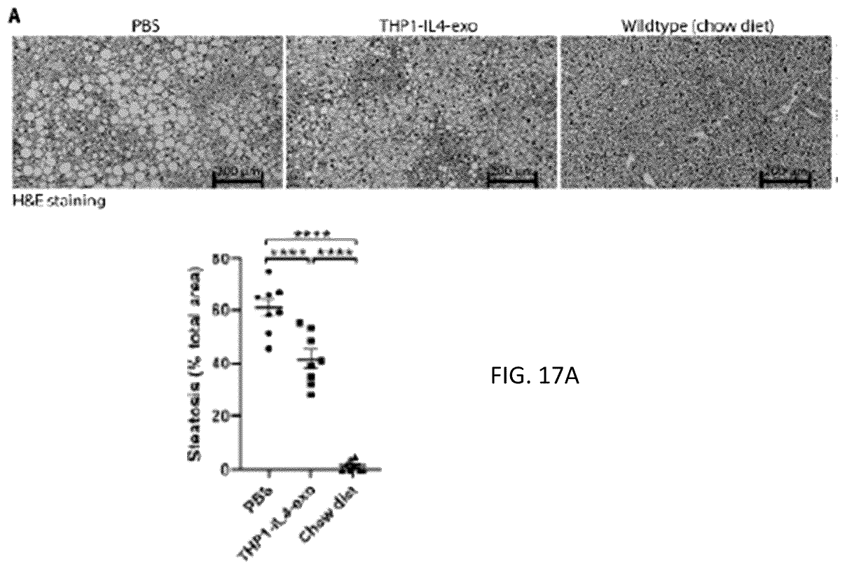

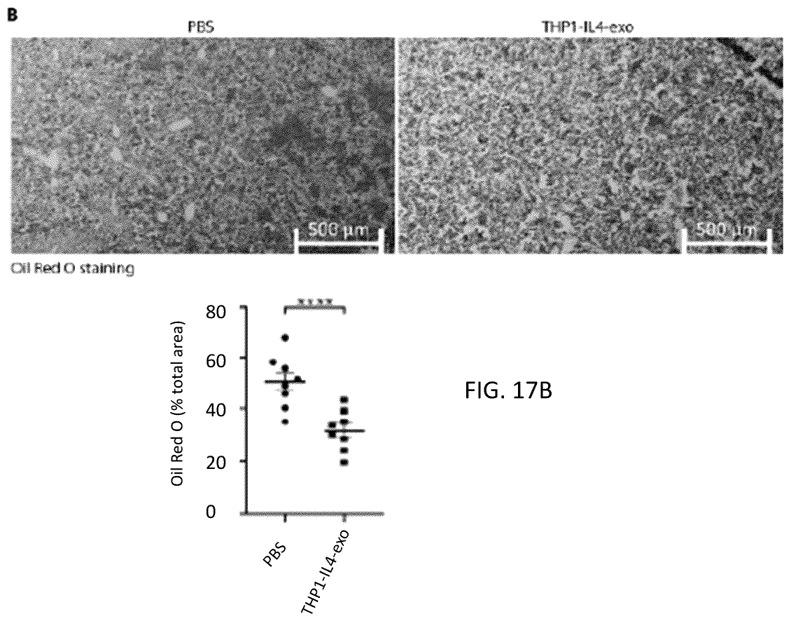

To determine whether benefits of controlled hematopoietic cell expansion and activation could extend to solid organs, leukocyte subsets in the aorta, liver, and epididymal white adipose tissues (eWAT) of mice infused with IL4-THP1-exo were examined. Enzymatic digestion of these tissues followed by flow cytometric detection of leukocytes revealed a marked reduction in CD45⁺ leukocytes in all three organs (FIGS. 7A-D, G). Subpopulation analyses further revealed lower numbers of total CD11b⁺ cells and CD11b⁺ F4/80⁺ macrophages in the aorta (FIGS. 7A, B). Moreover, within the aortic CD11b⁺ population, an expansion of Ly6C^{lo} monocytes and contraction of Ly6C^{hi} monocytes and neutrophils subpopulations was observed (FIGS. 7A, B). A similar reduction in CD45⁺ leukocytes was also observed in livers of mice infused with THP1-IL4-exo (FIGS. 7C, D). While there was no difference in the number of liver-associated CD11b^{lo} F4/80^{hi} Kupffer cells, there was a significant reduction in infiltrating CD11b^{hi} F4/80⁺ macrophages in the livers of mice infused with THP1-IL4-exo (FIGS. 7C, D). Furthermore, lower numbers of total CD11b⁺ cells were detected in the liver and, within this population, lower numbers of Ly6C^{hi} and neutrophils subpopulations in THP1-IL4-exo-infused mice (FIGS. 7C, D). An analysis of gene expression levels from RNA extracted from the livers of these mice also showed a decrease in the expression of Adgre1 (F4/80) and inflammatory markers associated with M1 macrophages (Tnf, Il1b, Mcp1, and Nos2) (FIG. 7F). In contrast, an increased expression of M2-associated markers (Arg1, Chil3, and Retnla) was noted (FIG. 7E). Lastly, histological assessments of the liver revealed reduced hepatocyte enlargement and Oil Red O retention in of liver sections taken from Western diet-fed Apoe^{h/h} Ldlr^{-/-} mice treated with THP1-IL4-exo (FIGS. 17A, B). Together, these data support the benefits of THP-IL4-exo in controlling the onset of diet-induced steatohepatitis.

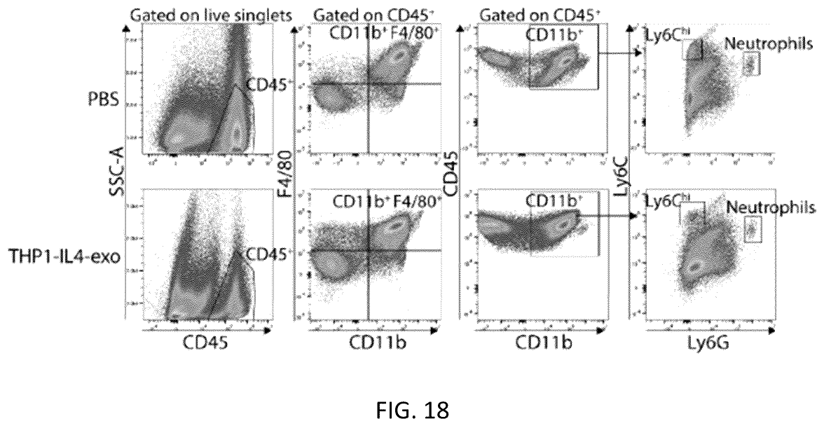

In spite of a lack of significant changes in weight gains (FIGS. 16C, D), the data show that THP1-IL4-exo infusions profoundly suppressed eWAT inflammation. Immunohistochemical analysis revealed reduced numbers of F4/80⁺ crown-like structures (CLSs) in eWAT derived from mice treated with THP1-IL4-exo as compared to those treated with PBS (FIG. 7F). Flow cytometric analysis of cellular digests from eWAT collected from Western diet-fed Apoe^{h/h} Ldlr^{-/-} mice showed a reduction in CD45⁺ leukocytes in mice infused with THP1-IL4-exo (FIGS. 18 and 7G). Subpopulation analyses further showed reduced numbers of total CD11b⁺ cells and CD11b⁺ F4/80⁺ macrophages in eWAT of these mice (FIGS. 18 and 7G). Among all eWAT-associated CD11b⁺ cells, fewer numbers of Ly6C^{hi} monocytes and neutrophils were detected (FIGS. 18 and 7G). Similar to the findings of liver tissue taken from these mice (FIG. 7E), gene expression analysis of eWAT RNA extracts showed a marked decrease in the expression of Adgre1 (F4/80) and M1 markers/pro-inflammatory cytokines (Tnf, Il1b, Mcp1, and Nos2) with a concomitant increase in M2/anti-inflammatory markers (Arg1, Chil3, and Retnla) (FIG. 7H).

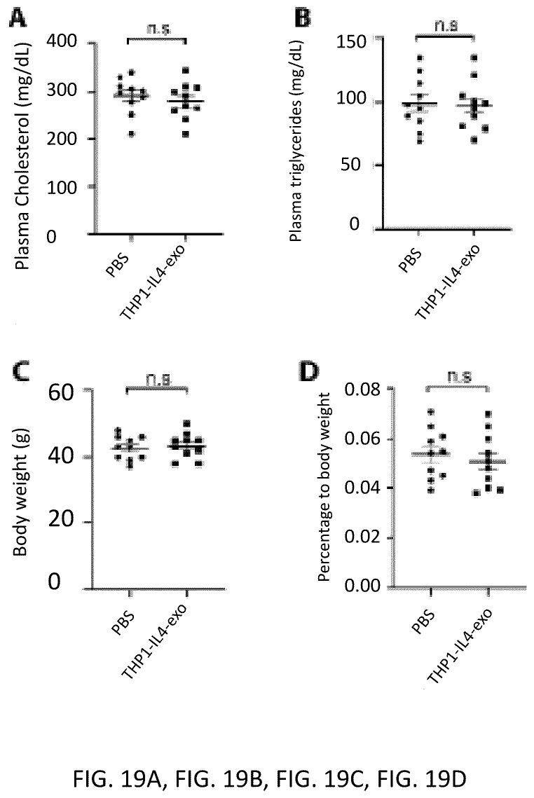

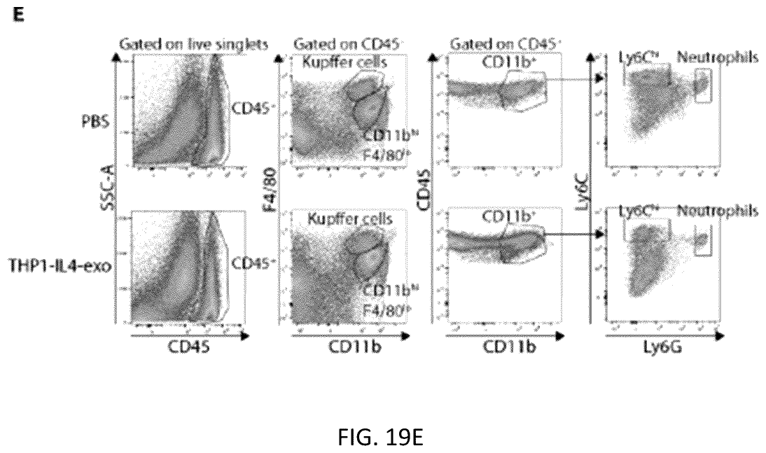

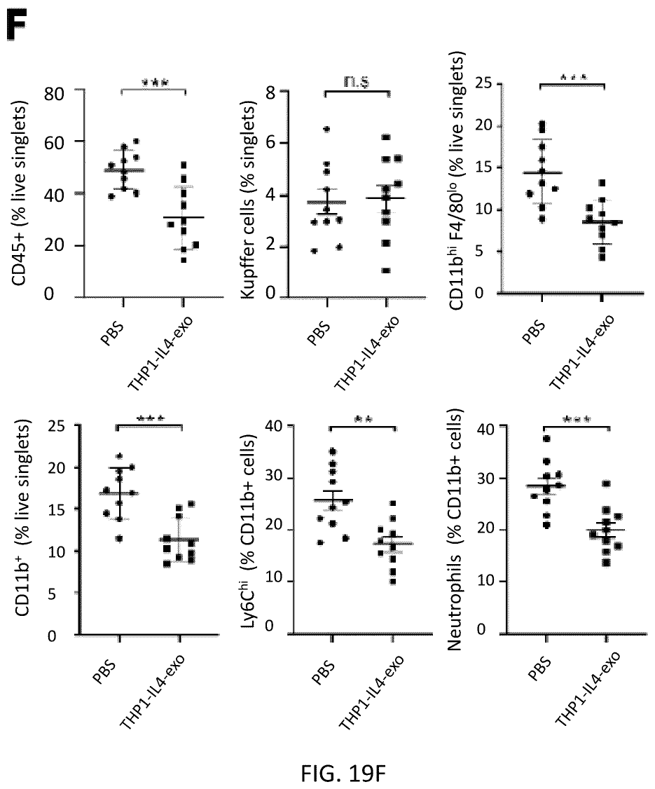

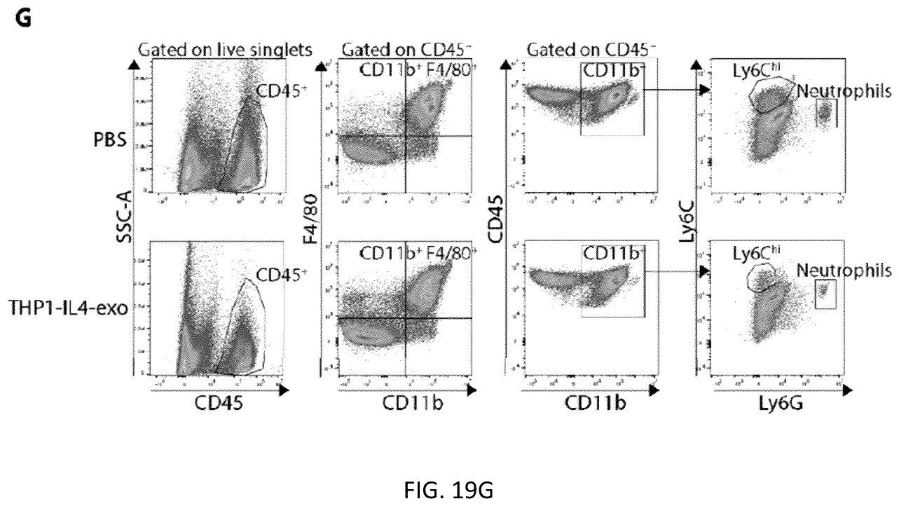

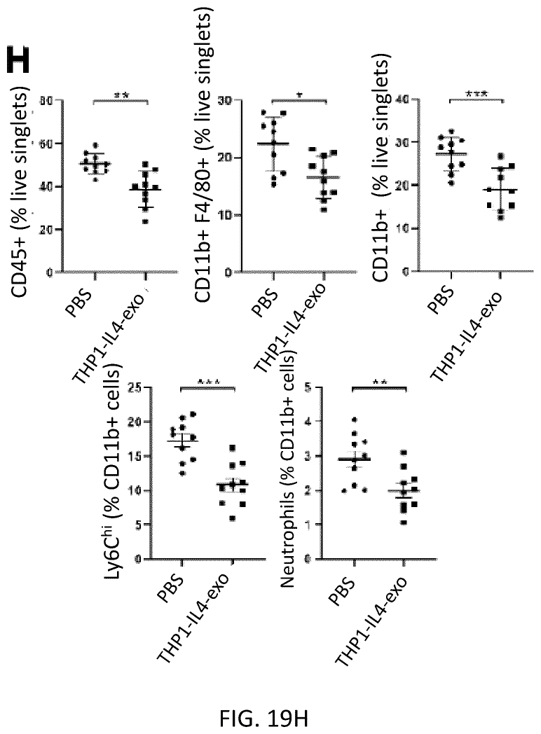

To confirm the efficacy of THP1-IL4-exo in controlling cardiometabolic inflammation in a second model system, 20-week-old Western-diet fed wildtype mice were repeatedly infused for 6 weeks while keeping them on the diet. These mice displayed significantly lower plasma cholesterol and triglycerides than Apoe^{h/h} Ldlr^{-/-} mice, averaging 300 mg/dL and 100 mg/dL respectively (FIGS. 19A-B). Despite lower blood lipids, the Western diet-fed wildtype mice displayed similar total body weights and eWAT fat-to-body ratio similar to Western diet-fed Apoe^{h/h} Ldlr^{-/-} mice, averaging 43 grams and 0.057 and 0.051 fat-to-body ratio for PBS-treated and THP1-IL4-exo-treated groups, respectively (FIGS. 19C-D). As observed with the Apoe^{h/h} Ldlr^{-/-} model, repeated infusions of THP1-IL4-exo suppressed the accumulations of CD45⁺ leukocytes, CD11b^{hi} F4/80^{lo} macrophages, neutrophils, and Ly6C^{hi} CD11b⁺ cells in the livers of wildtype mice fed this diet (Figures STE-F). A similar reduction of CD45⁺ leukocytes, macrophages, neutrophils, and Ly6C^{hi} CD11b⁺ cells was also detected in eWAT when compared to mice infused with PBS (FIGS. 19G-H). Collectively, the data demonstrate the efficacy of repeated THP1-IL4-exo infusions in exerting a profound control over chronic vascular, hepatic and adipose tissue inflammation that develops in response to Western diet feeding in wildtype mice and low-density lipoprotein receptor-deficient mice. These findings also support this treatment to drive an expansion of protective M2-like macrophages in these tissues.

viii. THP1-IL4-Exo Drive the Beiging of White Adipose Tissue that Improves Glucose Disposal and Insulin Resistance in Obese Mice

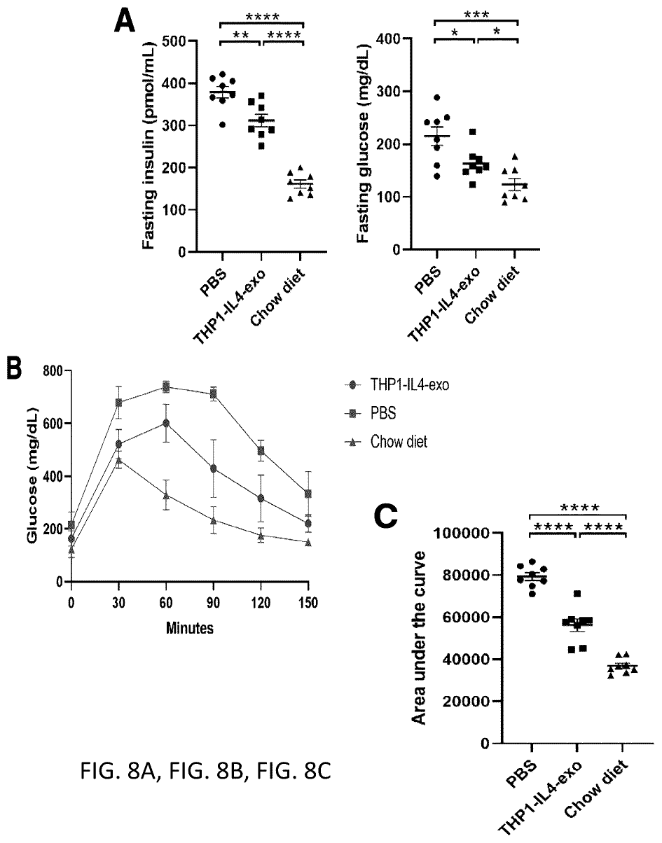

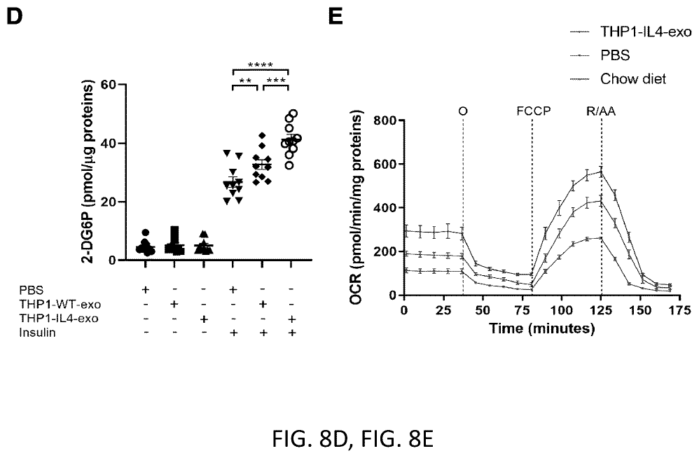

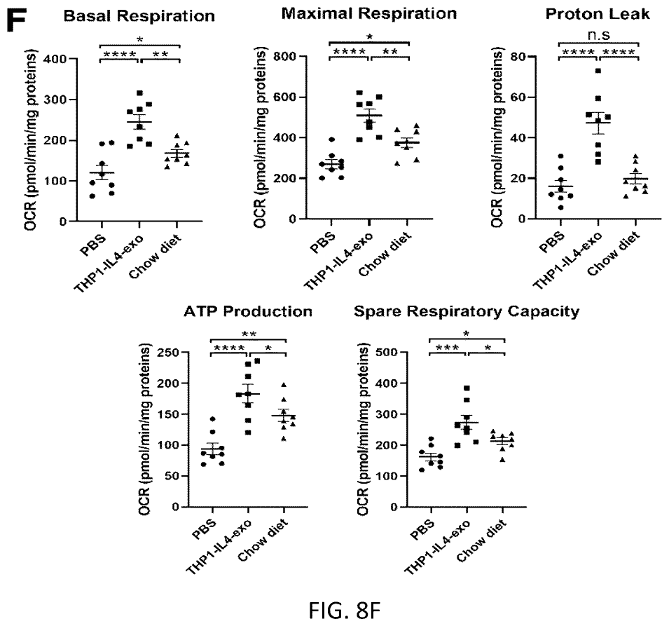

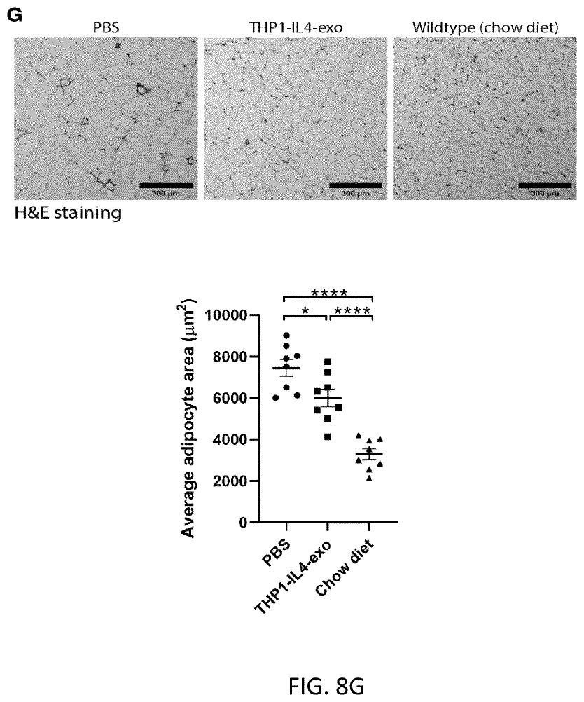

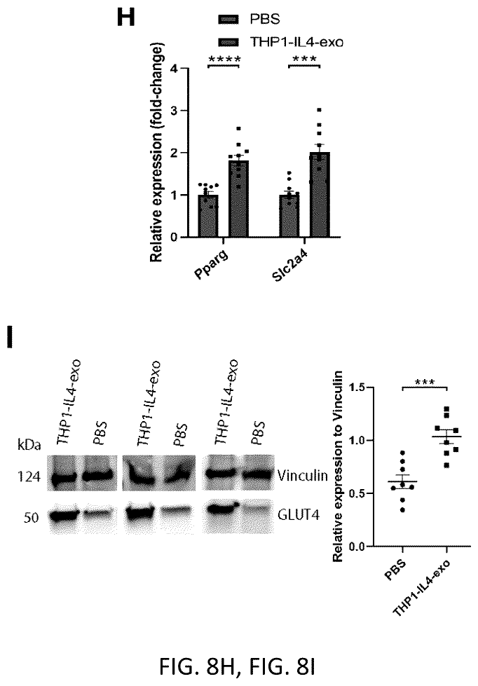

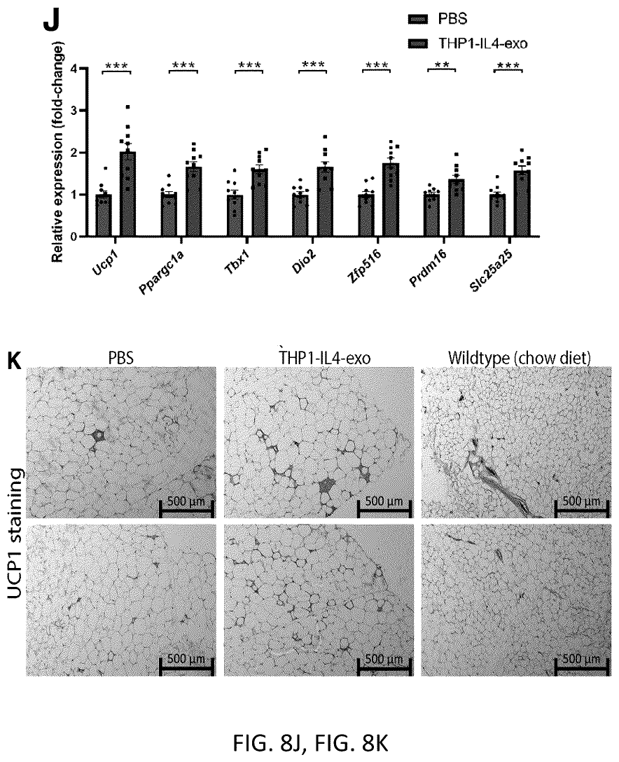

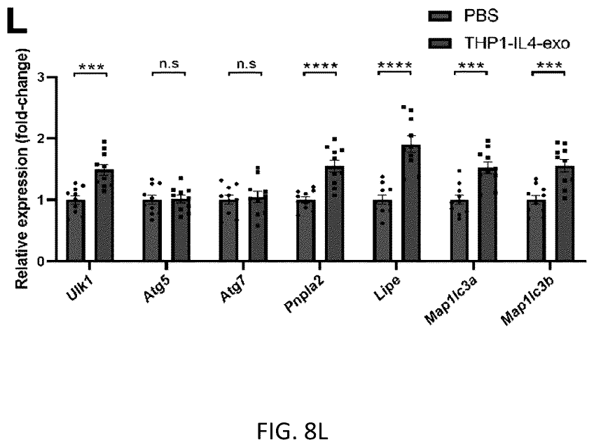

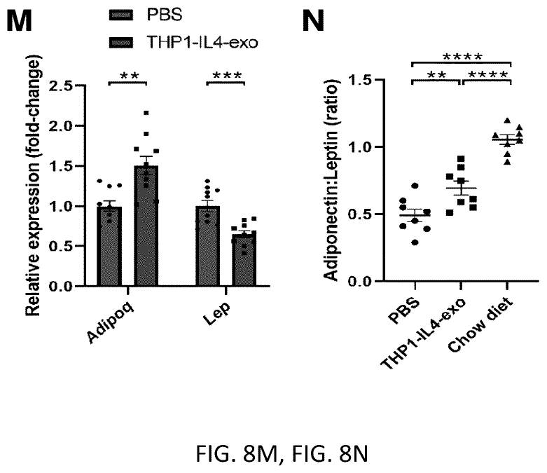

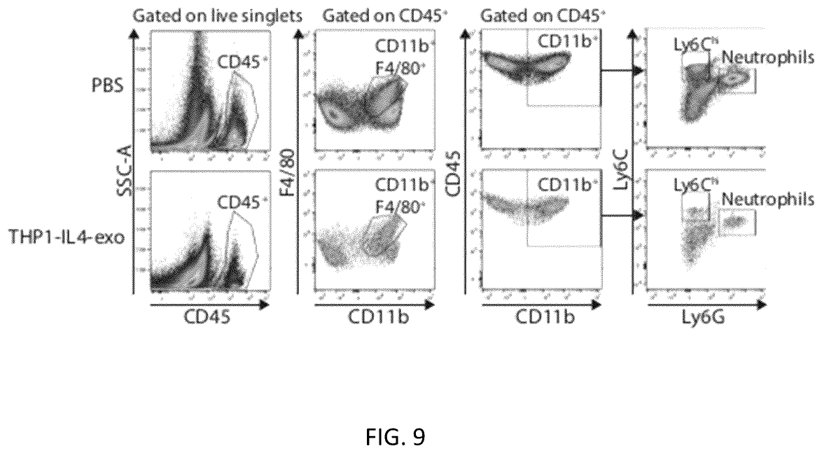

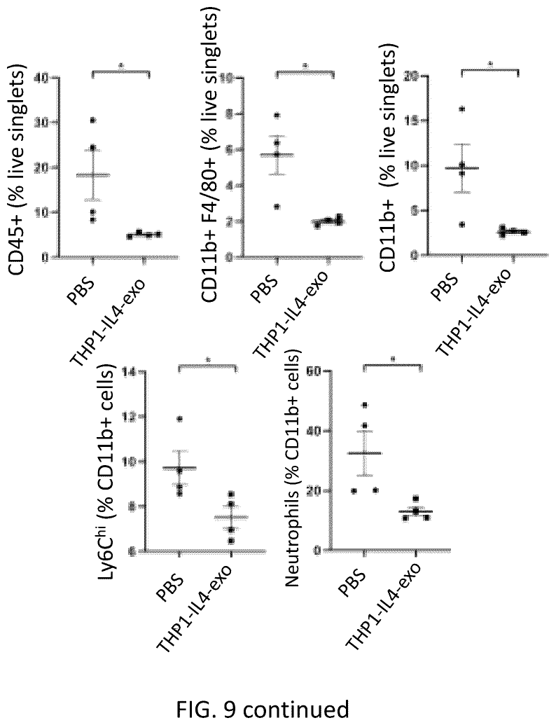

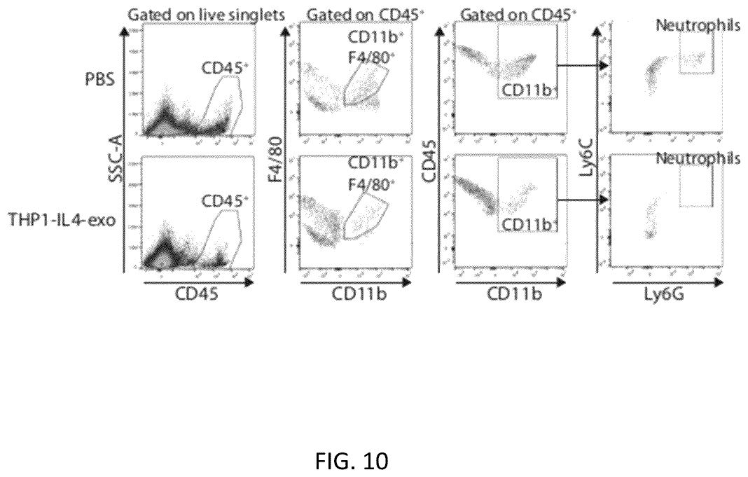

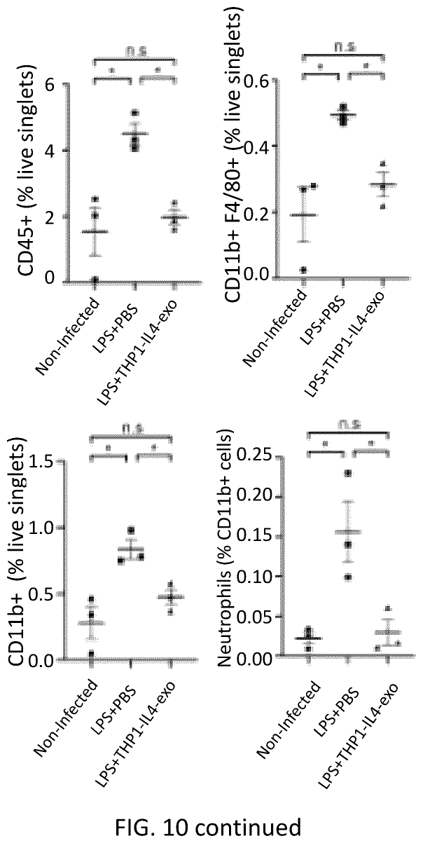

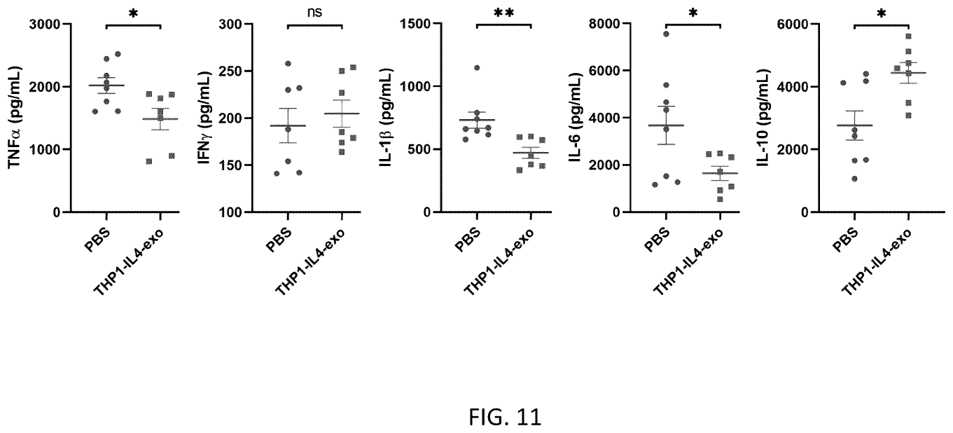

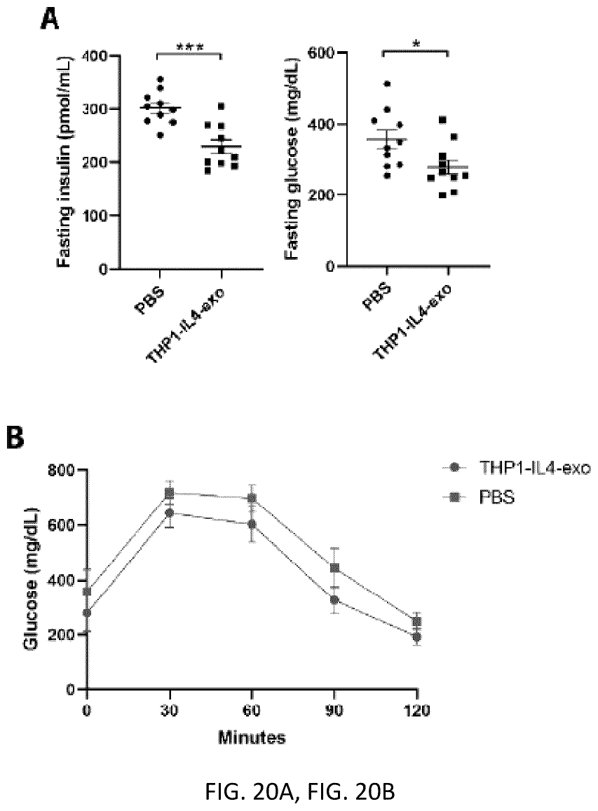

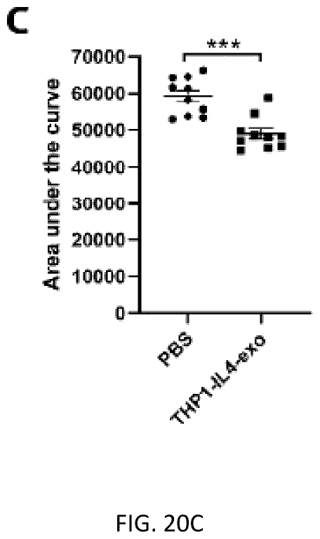

The in vivo findings so far provide compelling evidence demonstrating the efficacy of THP1-IL4-exo in suppressing inflammation in cardiometabolic tissue including eWAT. Based on the data demonstrating their capacity to drive the beiging of cultured adipocytes, it was wondered whether THP1-IL4-exo could protect hyperlipidemic Apoe^{h/h} Ldlr^{-/-} mice against obesity-induced insulin resistance and impaired glucose disposal. It was noted that 6 weeks of tri-weekly i.p. infusions substantially reduced fasting glucose and insulin levels in these mice (FIG. 8A). Furthermore, such treatments profoundly enhanced glucose disposal as measured by a glucose tolerance test (GTT) (FIGS. 8B, C). A correction of all of these metabolic parameters related to Type II diabetes were reproduced in the second mouse model using Western diet-fed wildtype mice infused with a similar course of THP1-IL4-exo (FIGS. 20A-C).

To investigate mechanisms responsible for improved glucose disposal observed in obese mice treated with THP1-IL4-exo, levels of 2-deoxyglucose (2-DG) uptake were examined in cultured 3T3-L1 adipocytes. Using this approach, increased 2-DG uptake was noted in 3T3-L1 adipocytes treated with insulin and THP1-IL4-exo (FIG. 8D). Interestingly, the use of THP1-WT-exo also increased 2-DG uptake in 3T3-L1 cells upon insulin stimulation, although to a lesser extent compared to THP1-IL4-exo treatment (FIG. 8D). To further interrogate metabolic benefits that THP1-IL4-exo contribute to adipose tissue, the OCR was measured in whole eWAT taken from Western diet-fed Apoe^{h/h} Ldlr^{-/-} mice using the Seahorse Mito Stress Assay. Paralleling the observed control of tissue inflammation, eWAT taken from THP1-IL4-exo-infused mice displayed a profound increase in OXPHOS activity. This was revealed by an elevated basal and maximal respiration associated with a higher proton leak and ATP production as compared to PBS-infused mice (FIGS. 8E, F). Remarkably, eWAT from Western diet-fed mice infused with THP1-IL4-exo displayed greater OXPHOS activity than eWAT from control chow-fed mice (FIG. 8E, F), demonstrating the capacity of these exosomes to profoundly upregulate metabolic activity in eWAT even when challenged by a high-fat, high-sucrose diet. Furthermore, the benefits that THP1-IL4-exo infusions conferred to improve energy metabolism and white adipose tissue homeostasis mitigated adipocyte hypertrophy in eWAT of Western diet-fed Apoe^{h/h} Ldlr^{-/-} mice (FIG. 8G), corroborating with the benefits of M2 macrophage expansion in white adipose tissues of obese mice.

Lastly, the expression levels of genes responsible for energy metabolism were measured in eWAT tissue extracts. Consistent with the in vitro studies, an increase in the mRNA expression of Pparg, as well as the mRNA and protein products of its target gene, Slc2a4 (GLUT4) were observed in eWAT extracts of THP1-IL4-exo-infused mice (FIGS. 8H, I). An upregulation of genes involved in eWAT beiging and thermogenesis were also detected that included: Ucp1, Ppargc1a, Tbx1, Dio2, Zfp516, Prdm16, and Slc25a25 (FIG. 8J). Immunohistochemical analysis revealed increased levels of UCP1 in WAT of mice treated with THP1-IL4-exo as compared to PBS-treated mice (FIG. 8K). The altered expression pattern of these genes and protein products supports the observed increase in OXPHOS activity in eWAT derived from THP1-IL4-exo-infused mice in FIGS. 8E-F. Remarkably, an upregulated expression in genes associated with lipophagy (Ulk1, Pnpla2, Lipe, Map1lc3a, and Map1lc3b) was observed in eWAT of THP1-IL4-exo-infused mice (FIG. 8L). Together, these findings are consistent with the in vitro data from cultured 3T3-L1 adipocytes in FIGS. 5F-G. In line with data from the in vitro studies from cultured 3T3-L1 adipocytes in FIG. 5H, an increase in the gene expression of adiponectin (Adipoq) and reduced expression of Leptin (Lep) was also found in WAT of mice treated with THP1-IL4-exo (FIG. 8M). This finding parallels a nearly two-fold increase in the plasma adiponectin: leptin ratio detected in Western diet-fed mice infused with THP1-IL4-exo (FIG. 8N), supporting improved adipose tissue function in response to this treatment.

### 3. Discussion

Findings from this study reveal that exosomes produced by the human monocytic cell line THP-1 are effective in controlling cardiometabolic inflammation along with the onset of diabetes in mice fed a Western high fat-diet. It was also shown that an exposure to IL-4 is critical for THP-1 cells to produce exosomes capable of controlling inflammation in the liver, adipose tissue and aorta of obese diabetic mice. While benefits of the exosome treatments likely included their potent control of hematopoiesis in the bone marrow and spleen as was recently reported with studies of M2-like murine macrophage exosomes, they also resulted from profound shifts in cellular energy metabolism that led to increased mitochondrial respiration observed both in ex vivo adipose tissue as well as in cultured adipocytes and macrophages. A common signaling cascade observed in both cultured cell types and circulating Ly6Chi monocytes was an upregulation of PPARγ expression and reduced levels of miR-33-5p that together increased the expression of genes associated with lipophagy and cellular lipid elimination. Augmented aerobic bioenergetic activity in adipose tissue of mice treated with THP1-IL4-exo was accompanied by the beiging of white adipose tissue that improved glucose clearance and normalized resting blood glucose and insulin levels in obese mice. Together, these findings highlight cell-signaling properties through which THP1-IL4-exo rewire energy metabolism in target cells to drive mitochondrial respiration and introduce these exosomes as possible treatments for cardiometabolic inflammation and Type II diabetes in obesity.

Exosomes produced by macrophages are increasingly recognized as mediators of intercellular signaling in cardiovascular inflammation and diabetes. Furthermore, accumulating evidence points to the activation status of macrophages as a source of microRNA cargo diversity in secreted exosomes that contribute to cellular signaling in numerous pathologies. Indeed, macrophages exposed to inflammatory cytokines and metabolic stressors, including LPS, oxidized low-density lipoprotein, and high glucose, have been documented to produce exosomes enriched with microRNAs that contribute to cardiovascular inflammation, insulin resistance, and atherosclerosis acceleration. In contrast, macrophages exposed to anti-inflammatory cytokines enriched exosomes with microRNAs that resolved systemic inflammation and atherosclerosis in mice with hyperlipidemia and restored insulin sensitivity in obese mice. Findings from this study of THP1-IL4-exo support the value of M2-like macrophage exosomes to control chronic inflammatory disorders caused by excessive energy storage. Furthermore, the findings introduce the capacity for THP1-IL4-exo to modulate mitochondrial bioenergetics in macrophages and adipocytes to drive beiging in white adipose tissue, leading to the resolution of cardiometabolic inflammation in obese diabetic mice. The findings thus provide translational relevance for exosomes produced by a well-defined human macrophage cell line that could be suitable for human therapy.

The molecular source of signaling through which THP1-IL4-exo altered metabolic properties of target cells, while not directly examined in this study, likely derived in part from a delivery of microRNA cargo that include miR-99a-5p/146b-5p/378a-3p. Indeed, the findings support functional transfer of these microRNAs as they were enriched in THP1-IL4-exo and all recipient cells tested. Furthermore, miR-21-5p was noted to be highly enriched in both the exosomes and their target cells, consistent with data reporting it as the most abundant microRNA in exosomes produced by macrophages irrespective of their stimulation with IL-4. Futures studies will be required to validate functional transfer of microRNA and other bioactive cargo enriched in THP1-IL4-exo that could include long non-coding RNA and even metabolites of mitochondrial respiration as sources of cellular regulation. Regardless of how THP1-IL4-exo provoked an upregulation of all four of these microRNAs in their target cells, their functional significance was striking with respect to the observed rewiring of energy metabolism in cultured cells and cardiometabolic tissue in obese mice. The mechanistic basis for these benefits likely derived from the exosomes' targeted control of mRNA gene expression.

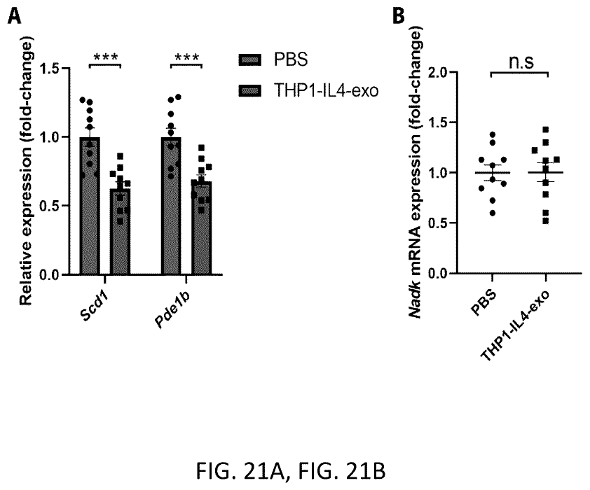

Based on prior findings, increased cellular miR-99a-5p likely contributed to control TNF-α mRNA levels to suppress M1-like macrophage marker genes that included iNOS which plays a central role in promoting insulin resistance and atherosclerosis. Furthermore, exosomal miR-99a is likely beneficial in improving glucose tolerance, insulin sensitivity and preserved liver function by preventing cellular oxidative stress via NOX4 mRNA control as reported in models of obesity and fatty liver disease. Similarly, increased cellular miR-378a-3p levels likely exerted numerous metabolic benefits, including the control of macrophage proliferation and obesity in the cohorts of Western diet-fed mice. While not directly examined in this study, exosomal miR-378a-3p could have exerted a profound increase in lipolysis and the expansion of brown adipose tissue and its thermogenic properties by downregulating Scd1 and Pde1b in adipocytes and white adipose tissue of the mice (FIGS. 15C and 21A), contributing to normalizing blood glucose and insulin levels in obese mice. Furthermore, based on a recent report of miR-378a activity in white adipose tissue, its exosomal delivery to this tissue likely served a central role in promoting beiging and improved glucose and insulin signaling in the obese mice injected with THP1-IL4-exo. Favorable metabolic reprograming in target tissues likely also arose from an upregulated expression of miR-146b-5p. Indeed, the exosomal delivery of this microRNA in controlling inflammatory activity by reducing TLR/NF-κB signaling in macrophages and HSPC that attenuated atherosclerosis progression 17. Interestingly, the control of macrophage activation via miR-146a has been reported to control metabolic dysfunction in diet-induced obesity48, a pathway that could be shared by its homolog miR-146b which will require further investigation.

Above all microRNAs found to be upregulated in BMDM and 3T3-L1 adipocytes treated with THP-1 exosomes, miR-21-5p likely served to substantially improve cardiometabolic inflammation and attenuate atherosclerosis in the cohorts of obese mice. Indeed, miR-21-5p is well recognized as a central regulator of macrophage metabolic reprograming and cellular activity in atherosclerosis control. MiR-21-5p also protects against insulin resistance and improves glucose uptake in adipocytes by modulating the PTEN-AKT pathway resulting in increased mobilization of GLUT4 upon insulin stimulation. Furthermore, recent findings have reported its potent capacity to improve metabolic activity, WAT beiging, and BAT induction in obese mice through the modulation of VEGF-A, p53, and TGFβ1 signaling pathways. Taken together, the data indicate a cellular enrichment of microRNA carried by THP1-IL4-exo as a key element to explain their control of cardiometabolic inflammation in obese mice.

Central to the metabolic signaling properties exerted by THP1-IL4-exo was the profound reduction in miR-33-5p that the exosomes induced in cultured BMDM, 3T3-L1 adipocytes, and circulating Ly6Chi monocytes. Reduced levels of miR-33-5p can in part be explained by the downregulation of its host, Srebf2, in addition to the homolog, Srebf1, which are both repressed by PPARγ. The findings therefore support the importance of PPARγ induction in response to THP1-IL4-exo treatment as a major driver of metabolic reprogramming in target cells by downregulating SREBP-1/2 gene expression and reducing levels of miR-33-5p, a critical energy metabolism hub central to the control of lipid synthesis and metabolism. An induced expression of PPARγ likely arose by a functional transfer and increased cellular levels of miR-99a and miR-378a that have both been reported to drive the gene expression of PPARγ42, 46, 57-58. Taken together, these findings reveal novel insights into molecular properties through which M2-like macrophage exosomes control the reprogramming of cellular metabolism via the upregulation of microRNAs that drive the expression of PPARγ and subsequent reduction of miR-33-5p.

While the findings support those from a recent study demonstrating benefits of antagonizing miR-33-5p to improve thermogenesis and adipose tissue beiging, they also introduce M2-like macrophage exosome as effectors of these metabolic processes. In testing the effects of miR-33-5p downregulation conferred by THP1-IL4-exo, a marked increase in its target genes involved in adipocyte beiging was observed in both cultured 3T3-L1 adipocytes and WAT of Western diet-fed obese mice. In particular, a profound increase in the transcriptional regulators and coregulators Zfp516, Ppargc1a, and Prdm16, as well as the enzyme Type II iodothyronine deiodinase Dio2, and the calcium-binding mitochondrial carrier protein involved in mitochondrial function Slc25a2529 were seen. The enhanced expression of these genes likely contributed to the observations of increased uncoupling protein 1 (UCP1) gene and protein expression that is recognized as a key regulator in thermogenesis and adipocyte beiging. This pattern of gene and protein expression likely led to an observed increase in mitochondrial respiration and subsequent OXPHOS activity in both cultured adipocytes and WAT, a critical process during thermogenesis and adipocyte beiging. The improvements in mitochondrial function caused by the cellular uptake of THP1-IL4-exo likely also stemmed from their ability to control mitochondrial permeability by restricting the opening of the transition pore as a result of their capacity to reduce oxidative stress as shown in FIGS. 2E and 5C.

Such functional benefits in the adipose tissue were likely also driven from the observed upregulation of genes associated with lipophagy, lipid catabolism and cholesterol efflux, all of which are controlled by miR-33-5p. In particular, an increase in expression of genes associated with lipophagy, including Ulk1, Pnpla2, Lipe, Map1lc3a, and Map1lc3b, in BMDM, 3T3-L1 adipocytes, and eWAT of mice treated with THP1-IL4-exo was found. Among these genes, Ulk1 (Unc-51 Like Autophagy Activating Kinase 1), Map1lc3a (Microtubule Associated Protein 1 Light Chain 3 Alpha), and Map1lc3b (Microtubule Associated Protein 1 Light Chain 3 Beta) are known inducers of autophagy34. In addition, Pnpla2 (Adipose Triglyceride Lipase) and Lipe (Hormone Sensitive Lipase) are known to induce fatty acid and cholesterol catabolism in macrophages and adipocytes during cardiovascular and metabolic inflammation.

Beyond serving as a checkpoint for miR-33-5p expression, PPARγ induction by THP1-IL4-exo exposure increased the expression of lipid mobilizing and efflux genes including Abca1 and Apoe, as well as the insulin-dependent glucose transporter GLUT4 (Slc2a4), in cells cultured in vitro and those tested in mice. Indeed, an upregulation of Abca1 observed in BMDM and circulating Ly6Chi monocytes could have directly derived from a reduction of miR-33-5p, resulting in a dual mode of transcriptional regulation29, 62. Interestingly, the expression of Abcg1 was not as highly upregulated as Abca1, despite being both targets of miR-33-5p, indicating that Abcg1 may be subject to post-transcriptional regulation in macrophages and monocytes. Regardless, the profound decrease in neutral lipids detected in both BMDM and 3T3-L1 adipocytes treated with THP1-IL4-exo support a functional relevance to the observed upregulation of genes involved in lipid catabolism that likely serve as biofuels to satisfy demands of increased mitochondrial respiration and efflux of free cholesterol that would otherwise be deleterious. Indeed, enhanced cellular cholesterol elimination observed in macrophages and monocytes has long been noted to control monocytosis and hematopoiesis, including in diabetic mice during atherosclerosis regression in response to miR-33-5p inhibition. Together, the observations demonstrate that THP1-IL4-exo are effective in activating pathways critical for the catabolism and efflux of fatty acids and cholesterol in macrophages and adipocytes that are controlled by miR-33-5p.

A critical element for improving metabolic health in the treatment of obesity is a restoration of balance in adipokine expression, particularly in maintaining the adiponectin: leptin ratio at a level of 1.0 or higher. Such pattern of adipokine expression enhances cold-induced beiging of white adipose tissue, fosters M2 macrophage proliferation and restores white adipocyte homeostasis. Remarkably, THP1-IL4-exo treatments induced this beneficial effect by upregulating adiponectin (Adipoq), while concomitantly downregulating leptin (Lep) in adipocytes in cultured cells and in vivo in adipose tissues of obese Western-diet fed mice infused with THP1-IL4-exo. This profound shift in endocrine function likely contributed to normalize levels of insulin and glucose in obese mice fed a Western high-fat diet.

These findings are in line with those that revealed the utility of miRNA delivery in diabetes control. Indeed, miR-99a-5p/378a-3p/21-5p that have all been reported to improve cellular aspects of metabolism in diabetes are all enriched in THP1-IL4-exo and in their target cells. Furthermore, while the data generally support beneficial properties reported for M2-like BMDM exosomes in improving insulin sensitivity in obese high fat diet-fed mice, the mechanisms to explain such benefits differ. Indeed, BMDM exposed to a combination of IL-4 and IL-13 led to exosomes enriched with miR-690 that restored insulin sensitivity in obese mice via the control of a Nadk relevant pathway involved in macrophage inflammatory response and insulin signaling. As miR-690 is a mouse-specific microRNA not carried by THP1-IL4-exo, it is not surprising that the data of BMDM and 3T3-L1 adipocytes treated with THP1-IL4-exo did not reveal changes in the expression of Nadk (FIG. 21C). These results indicate that THP1-IL4-exo exert protective properties that do not overlap with the mechanism identified by the study of exosomes derived from IL-4/IL-13-treated primary mouse macrophages in controlling insulin resistance in obese mice.

In conclusion, these findings unveil a plethora of benefits exerted by IL-4-stimulated THP-1 macrophage exosomes in improving adipokine homeostasis and rewiring energy metabolism in target cells including macrophages and adipocytes to control the onset of cardiometabolic inflammation during obesity-driven diabetes. Together, the findings provide compelling evidence for the broad and potent capacity of THP-IL4-exo to resolve inflammation and enhance energy homeostasis in adipose tissue and hematopoietic compartments. The data therefore support future studies of THP1-IL4-exo in large mammals to determine their suitability for the treatment of obesity-driven cardiometabolic disease and type II diabetes.

### 4. Materials and Methods

i. Animal Studies

In vivo studies were conducted using hypomorphic Apoe^{h/h} Ldlr^{-/-} mouse strain by breeding Ldlr^{-/-} mice on a C57BL/6J background (Jackson Laboratories, USA) to hypomorphic (Apoe^{h/h}) mice⁶⁶. Ten-week-old male Apoe^{h/h} Ldlr^{-/-} or C57BL/6 wildtype mice were fed a Western diet (Research Diets, USA) for 10 weeks before being randomly assigned to be infused with THP1-IL4-exo or PBS as control for 6 weeks while remaining on the Western diet for the duration of the study (n=8-10 for each treatment group, pooled from two to three cohorts of mice). Data collection and analyses were conducted in a blinded-fashion. All mice were housed and bred in specific pathogen-free conditions in the Animal Research Facility at the San Francisco Veterans Affairs Medical Center. All animal experiments were approved by the Institutional Animal Care and Use Committee at the San Francisco Veterans Affairs Medical Center.

ii. Cell Culture

The human monocytic cell line (THP-1) was purchased from the UCSF Cell and Genome Engineering Core (CGEC) as an authenticated stock. Cells were cultured in RPMI 1640 medium (Corning, USA) supplemented with 10% fetal bovine serum (GIBCO, USA), 1% GlutaMAX (GIBCO, USA), and 1% penicillin-streptomycin (GIBCO, USA). THP-1 cells were grown and expanded in suspension in a T-75 flask (Fisher Scientific, USA) until a density of 1×10⁶ cells/mL. Cells were then seeded to 10-cm plates (Corning, USA) at a density of 4×10⁶ cells/plate and differentiated into macrophages by culturing in 25 ng/mL phorbol 12-myristate 13-acetate (PMA) (Fisher Scientific, USA) for 48 hours. Cells were then cultured in PMA-free media for an additional 48 hours and subsequently exosomes-free media with or without recombinant human IL-4 (Peprotech, USA) for exosome production.

For in vitro experiments, THP-1 cells were seeded to a 12-well plate (Corning, USA) at a density of 4×10⁵ cells/well and differentiated into macrophages by culturing in 25 ng/ml phorbol 12-myristate 13-acetate (PMA) (Fisher Scientific, USA) for 48 hours. Cells were then cultured in PMA-free media for an additional 48 hours. Fresh culture media was then added with 2×10⁹ or 4×10⁹ particles/mL of exosomes or equal volume of phosphate buffered saline (PBS) (Corning, USA) as control and cultured for 24 hours prior to analysis.

The 3T3-L1 preadipocyte cell line was purchased from the UCSF CGEC as authenticated stock. Cells were cultured in Dulbecco's Modified Eagle's Medium (DMEM) (Corning, USA) supplemented with 10% fetal bovine serum (GIBCO, USA), 1% GlutaMAX (GIBCO, USA), and 1% penicillin-streptomycin (GIBCO, USA). To differentiate into adipocytes, 3T3-L1 cells were seeded into a 12-well culture plate (Corning, USA) at a density of 4×10⁴ cells/well and allowed to grow until fully confluent. The cells were then incubated as a confluent culture for 48 hours and subsequently cultured in Differentiation Medium containing DMEM supplemented with 10% fetal bovine serum (GIBCO, USA), 1.0 μM dexamethasone (Sigma Aldrich, USA), 0.5 mM methylisobutylxanthine (IBMX) (Sigma Aldrich, USA), and 1.0 ug/mL bovine insulin (Sigma Aldrich, USA), 1% GlutaMAX (GIBCO, USA), and 1% penicillin-streptomycin (GIBCO, USA) for an additional 48 hours. The cells were then changed into Adipocyte Maintenance Medium containing DMEM supplemented with 10% fetal bovine serum (GIBCO, USA), 1.0 ug/mL bovine insulin (Sigma Aldrich, USA), 1% GlutaMAX (GIBCO, USA), and 1% penicillin-streptomycin (GIBCO, USA). The Adipocyte Maintenance Medium was changed every 48-72 hours and cells were fully differentiated by 15 days of induction. Differentiated adipocytes were then cultured with 4×10⁶ particles/mL of exosomes or equal volume of PBS (Corning, USA) as control for 24 hours before being collected for analysis. In some experiments, 4×10⁶ particles/mL of exosomes or equal volume of PBS (Corning, USA) were given every two days after the cells were induced in Differentiation Medium.

Murine bone marrow derived macrophages (BMDM) were obtained as previously described¹⁷⁻¹⁸. Briefly, bone marrow cells were flushed from the tibia and femurs of 6- to 12-week-old male C57BL/6J mice. Cells were cultured in DMEM (Corning, USA) supplemented with 10% fetal bovine serum (GIBCO, USA), 1% GlutaMax (GIBCO, USA), and 1% penicillin-streptomycin (GIBCO, USA) and differentiated with 25 ng/ml mouse M-CSF (Peprotech, USA) for 6 days. BMDM were seeded into 12-well culture plates (Corning, USA) at a concentration of 3×10⁵ cells/well and stimulated with 2×10⁹ or 4×10⁹ particles/mL of exosomes or equal volume of PBS (Corning, USA) as control for 24 hours before collected for analysis.

iii. THP-1 Macrophage Exosomes Isolation and Nanoparticle Tracking Analysis

The exosome isolation and characterizations were performed in adherence to the MISEV2018 guidelines. THP-1 cells were seeded into 10-cm plates (Corning, USA) at a density of 4×10⁶ cells/plate as described above. PMA-free media was discarded after 48 hours of incubation. The cells were then washed once with PBS (Corning, USA) and cultured in exosomes-depleted media prepared by ultracentrifugation for 18 hours at 100,000×g (Type 45 Ti rotor, Beckman Coulter, USA) and filtration (0.2 μm) supplemented with or without 20 ng/mL human IL-4 (Peprotech, USA). After 24 hours of incubation, the conditioned media was collected. Exosomes were isolated from conditioned media using Cushioned-Density Gradient Ultracentrifugation (C-DGUC) as previously described¹⁷⁻²⁰. Briefly, the conditioned media was centrifuged at 400×g for 10 min at 4C to pellet dead cells and debris followed by centrifugation at 2000×g for 20 min at 4C to eliminate debris and larges vesicles. The supernatant was then filtered (0.2 μm) and centrifuged on a 60% iodixanol cushion (Sigma-Aldrich, USA) at 100,000×g for 3 hours (Type 45 Ti, Beckman Coulter, USA). OptiPrep density gradient (5%, 10%, 20% w/v iodixanol) was employed to further purify exosomes at 100,000×g for 18 hours at 4C (SW 40 Ti rotor, Beckman Coulter, USA). Afterward, twelve 1 mL fractions were collected starting from the top of the tube. Fraction 7 of the gradient was dialyzed in PBS with the Slide-A-Lyzer MINI Dialysis Device (Thermo Fisher Scientific, USA) and used for subsequent experiments and analyses. A flow diagram depicting the production and purification steps of THP-1 macrophage exosomes is shown in FIG. 13A.

Particles in Fraction 7 was subjected to size and concentration measurement by NanoSight LM14 (Malvern Instruments, Westborough, USA) at a 488-nm detection wavelength. The analysis settings were optimized and kept identical for each sample, with a detection threshold set at 3, three videos of 1 min each were analyzed to give the mean, mode, median, and estimated concentration for each particle size. Samples were diluted in 1:100 or 1:200 PBS and measured in triplicates. Data were analyzed with the NTA 3.2 software. All exosome samples were store at 4C and used within one month after isolation. Details relevant to exosome isolation and physical characterization data have been submitted to the EV-TRACK knowledgebase (EV-TRACK ID: EV200042).

iv. Labeling and In Vitro/In Vivo Tracking of THP-1 Macrophage Exosomes

Fluorescently detectable THP-1 exosomes were generated using PKH26 (Sigma-Aldrich, USA) or DiR (DiIC18 (7) (1,1′-Dioctadecyl-3,3,3′,3′ Tetramethylindotricarbocyanine Iodide) (Invitrogen, USA). The dye was added to the 3 mL iodixanol cushion layer containing exosome or to 3 ml of PBS to achieve a final concentration of 3.5 mM for PKH26 or 1 μM for DiR and incubated for 20 min at room temperature. Labeled exosomes and control were loaded below an iodixanol step gradient as described above in the exosome isolation section. Free dye and non-specific protein-associated dye were eliminated from labeled exosomes or from PBS control during this separation step. For in vitro experiments, BMDM or 3T3-L1 adipocytes were exposed to 4×10⁹ PKH26-labeled exosome for two hours, washed three times with PBS and imaged using a Zeiss Observer microscope. Fluorescence intensity of the PKH26-positive cells was measured by using ImageJ. For in vivo experiments, 20-week-old Western diet-fed Apoe^{h/h} Ldlr^{-/-} mice were infused i.p. with PBS or 1×10¹⁰ DiR-labeled exosomes for six hours. The mice were then extensively perfused with PBS. Blood, aortas, hearts, livers, eWAT, bones, spleen, lungs, brains, intestines, and kidneys were collected, imaged, and quantified for DiR fluorescence signal using the Odyssey Infrared Imaging System and Image Studio software.

v. Transmission Electron Microscopy

An assessment of exosome morphology was assessed by Electron microscopy by loading 7×10⁸ exosomes onto a glow discharged 400 mesh Formvar-coated copper grid (Electron Microscopy Sciences, USA). The nanoparticles were left to settle for two minutes, and the grids were washed four times with 1% Uranyl acetate. Excess Uranyl acetate was blotted off with filter paper. Grids were then allowed to dry and subsequently imaged at 120 kV using a Tecnai 12 Transmission Electron Microscope (FEI, USA).

vi. Protein Extraction and Immunoblotting

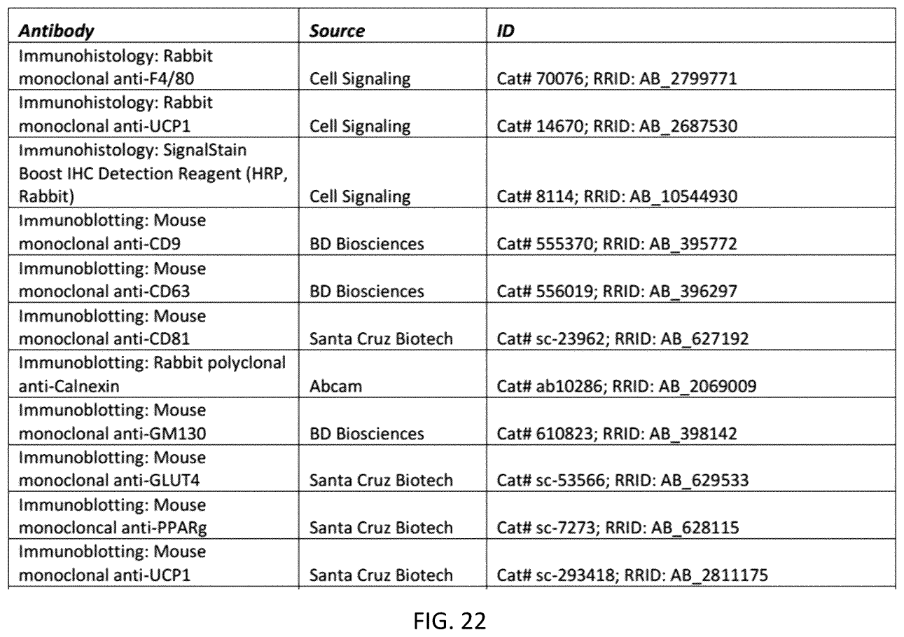

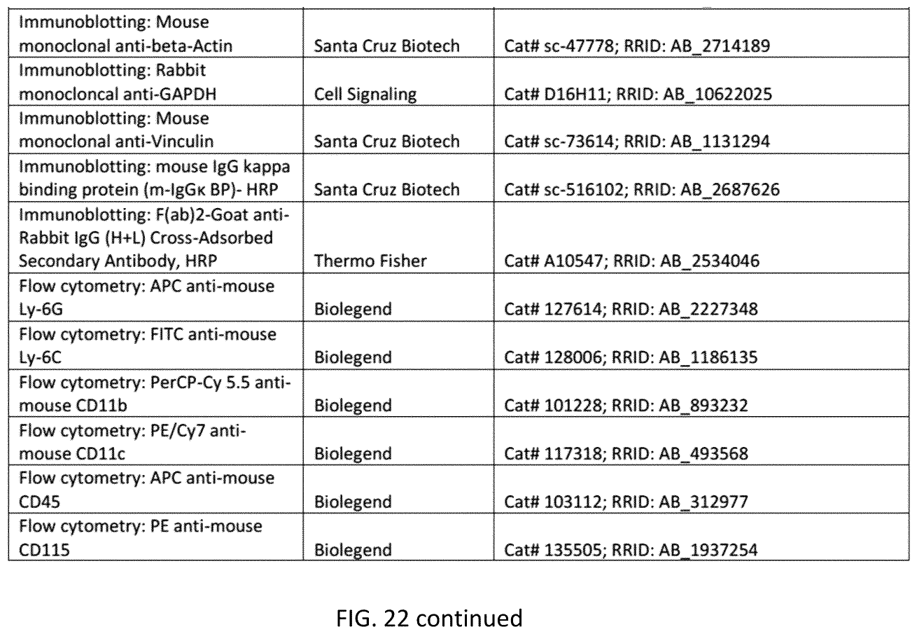

Each fraction of the C-DGUC purified exosomes (37.5 uL sample) was mixed with 12.5 mL of 4× Laemmli buffer (Bio-Rad, USA). For cell lysates, cells were lysed in RIPA Buffer (Cell Signaling, USA) containing complete, Mini, EDTA-free Protease Inhibitor Cocktail (Roche, Switzerland) and 1 mM PMSF (Cell Signaling, USA). For tissue lysates, 25 mg of eWAT were placed in RIPA Buffer (Cell Signaling, USA) containing complete, Mini, EDTA-free Protease Inhibitor Cocktail (Roche, Switzerland) and 1 mM PMSF (Cell Signaling, USA) and homogenized with a Tissue-Tearor. Protein concentrations were measured using Pierce BCA Protein Assay Kit (Thermo Fisher Scientific, USA). A total of 15 μg of proteins was diluted with PBS to 37.5 uL, then mixed with 12.5 uL 4× Laemmli buffer (Bio-Rad, USA). Samples were subsequently heated at 95C for 5 minutes. Samples were then loaded on a 10% SDS-PAGE gel and transferred onto PVDF membrane (Bio-Rad, USA). The membranes were blocked with 5% non-fat milk dissolved in PBS for one hour and then incubated with primary antibodies overnight at 4 C. Primary antibodies for exosomes markers include anti-CD9 (1:100, BD Biosciences, USA), anti-CD63 (1:100, BD Biosciences, USA), and anti-CD81 (1:100, Santa Cruz, USA). Primary antibodies for cell lysate markers include anti-Calnexin (1:500, Abcam, USA) and anti-GM130 (1:250, BD Biosciences, USA). Primary antibodies for 3T3-L1 adipocytes and eWAT tissued include anti-PPARγ (1:200, Santa Cruz, USA), anti-GLUT4 (1:200, Santa Cruz, USA), anti-UCP1 (1:200, Santa Cruz, USA), anti-b-Actin (1:1000, Santa Cruz, USA), anti-HSP70 (1:1000, Santa Cruz, USA), and anti-GAPDH (1:1000, Cell Signaling, USA), and—Vinculin (1:1000, Santa Cruz, USA). After 4 washes in PBS containing 0.1% Tween (PBST), membranes were incubated with corresponding HRP-conjugated secondary antibodies: anti-Mouse IgG-HRP (1:1000, Santa Cruz, USA) or anti-Rabbit IgG-HRP (1:1000, Thermo Fisher Scientific, USA) for 1 hour and washed in PBST. Signals were visualized after incubation with Amersham ECL Prime substrate and imaged using an ImageQuant LAS 4000. Quantification was analyzed using ImageJ. List of antibodies used is available in FIG. 22.

vii. RNA Extraction and Gene Expression Analysis Using qRT-PCR

Exosomes were treated with 0.4 mg/ml of RNase A/T1 Mix (Thermo Fisher Scientific, USA) for 20 min at 37C before RNA extraction. For RNA extraction from tissues, 100 mg of adipose tissue or 25 mg of liver tissue was placed into Qiazol Lysis Buffer (QIAGEN, Germany), the tissue was then homogenized using a Tissue-Tearor and spun at 12,000×g at 4C for 10 minutes. The supernatant was then collected for RNA extraction. Circulating Ly6C^{hi} monocytes were stained with the antibody cocktail indicated in the Primary cell preparation and purification section below and FACS sorted using a BD FACSAria II (BD Biosciences, USA) into Qiazol Lysis Buffer (QIAGEN, Germany).

Total RNA isolated from exosomes, cells, and tissues was extracted using Qiazol Lysis Buffer and purified using the RNeasy Mini Kit (QIAGEN, Germany) according to the manufacturer's protocol. RNA from liver and fat tissues were purified using on-column DNase digestion (QIAGEN, Germany) according to the manufacturer's protocol. RNA was quantified using Nanodrop or Quant-iT RiboGreen RNA Assay Kit (Thermo Fisher Scientific, USA) and reverse transcribed using the iScript Reverse Transcription Supermix (Bio-Rad, USA) for mRNA or the miRCURY LNA RT Kit (QIAGEN, Germany) for microRNA analysis. qPCR reactions were performed using the Fast SYBR Green Master Mix (Applied Biosystems, USA) for mRNA or the miRCURY LNA SYBR Green PCR Kit (QIAGEN, Germany) for microRNA and processed using a QuantStudio 7 Flex Real-Time PCR System. Ct values were normalized to the housekeeping gene Gapdh and B2m for mouse and Gapdh and Actb for human mRNA. For microRNA expression, UniSp6 was used as a spike-in control while U6 snRNA, miR-16-5p, and miR-21-5p (QIAGEN, Germany) were used as reference genes. All reactions were done in triplicate. Primers used for qRT-PCR are listed in Table 1.

TABLE 1
list of mRNA and microRNA primers used for qPCR applications.

| Species | Genes | Forward | Reverse |
|---|---|---|---|
| Human | Cd206 | AGCCAACACCAGCTCCTCAAG | CAAAACGCTCGCGCATTGTCCA |
| Human | Cd163 | ACATAGATCATGCATCTGTCATTG | ATTCTCCTTGGAATCTCACTTCTA |
| Human | TNFa | GGCGTGGAGCTGAGAGATAAC | GGTGTGGGTGAGGAGCACAT |
| Human | Il1b | CCACAGACCTTCCAGGAGAATG | GTGCAGTTCAGTGATCGTACAGG |
| Human | Cd86 | CTGCTCATCTATACACGGTTACC | GGAAACGTCGTACAGTTCTGTG |
| Human | Cd80 | CTGCCTGACCTACTGCTTTG | GGCGTACACTTTCCCTTCTC |
| Human | Gapdh | ATGGGGAAGGTGAAGGTCG | GGGGTCATTGATGGCAACAATA |
| Human | Actb | AGAGCTACGAGCTGCCTGAC | AGCACTGTGTTGGCGTACAG |
| Mouse | Arg1 | CTCCAAGCCAAAGTCCTTAGAG | AGGAGCTGTCATTAGGGACATC |
| Mouse | Chil3 | AGAAGGGAGTTTCAAACCTGGT | GTCTTGCTCATGTGTGTGTGTAAGT |
| | | | GA |
| Mouse | Retnla | CTGGGTTCTCCACCTCTTCA | TGCTGGGATGACTGCTACTG |
| Mouse | Tnf | CCCCAAAGGGATGAGAAGTTC | TGTGAGGGTCTGGGCCATAG |
| Mouse | Il1b | GCTCATCTGGGATCCTCTCC | CCTGCCTGAAGCTCTTGTTG |
| Mouse | Mcp1 | GCTACAAGAGGATCACCAGCAG | GTCTGGACCCATTCCTTCTTGG |
| Mouse | Pparg | TGTGGGGATAAAGCATCAGGC | CCGGCAGTTAAGATCACACCTAT |
| Mouse | Abca1 | ACCTGGAGAGAAGCTTTCAATGA | GTTCAGGTTGACACACTCCATGA |
| Mouse | Abcg1 | GACACCGATGTGAACCCGTTTC | GCATGATGCTGAGGAAGGTCCT |
| Mouse | Ulk1 | AAGTTCGAGTTCTCTCGCAAG | ACCTCCAGGTCGTGCTTCT |
| Mouse | Atg5 | TGTGCTTCGAGATGTGTGGTT | ACCAACGTCAAATAGCTGACTC |
| Mouse | Atg7 | TCTGGGAAGCCATAAAGTCAGG | GCGAAGGTCAGGAGCAGAA |
| Mouse | Pnpla2 | ATGTTCCCGAGGGAGACCAA | GAGGCTCCGTAGATGTGAGTG |
| Mouse | Lipe | GATTTACGCACGATGACACAGT | ACCTGCAAAGACATTAGACAGC |
| Mouse | Map1Ic3a | GACCGCTGTAAGGAGGTGC | CTTGACCAACTCGCTCATGTTA |
| Mouse | Map1Ic3b | TTATAGAGCGATACAAGGGGGAG | CGCCGTCTGATTATCTTGATGAG |
| Mouse | Apoe | CTCCCAAGTCACACAAGAACTG | CCAGCTCCTTTTTGTAAGCCTTT |
| Mouse | Srebf1 | TGACCCGGCTATTCCGTGA | CTGGGCTGAGCAATACAGTTC |
| Mouse | Srebf2 | GCAGCAACGGGACCATTCT | CCCCATGACTAAGTCCTTCAACT |
| Mouse | Slc2a4 | ACACTGGTCCTAGCTGTATTCT | CCAGCCACGTTGCATTGTA |
| Mouse | Ppargc1a | TATGGAGTGACATAGAGTGTGCT | GTCGCTACACCACTTCAATCC |
| Mouse | Tbx1 | GTCAAGGCTCCGGTGAAGAAG | GCTGATTGAACTCGTCCCACA |
| Mouse | Dio2 | ATGGGACTCCTCAGCGTAGAC | ACTCTCCGCGAGTGGACTT |
| Mouse | Zfp516 | CAGCCCTACTAAGAGCACCTC | CAGGGTGACATAGCTGCACAG |
| Mouse | Prdm16 | CCACCAGCGAGGACTTCAC | GGAGGACTCTCGTAGCTCGAA |
| Mouse | Slc25a25 | TGACCATCGACTGGAACGAGT | TCACCGACATCGAAGATCGTC |
| Mouse | Adipoq | TGTTCCTCTTAATCCTGCCCA | CCAACCTGCACAAGTTCCCTT |
| Mouse | Lep | GTGGCTTTGGTCCTATCTGTC | CGTGTGTGAAATGTCATTGATCC |
| Mouse | Scd1 | TTCTTGCGATACACTCTGGTGC | CGGGATTGAATGTTCTTGTCGT |
| Mouse | Pde1b | AGTTCCGAAGCATCGTGCAT | CTTGAGACAGTTGTGGACTGC |
| Mouse | Gapdh | TGAAGCAGGCATCTGAGGG | CGAAGGTGGAAGAGTGGGAG |
| Mouse | B2m | CTGCTACGTAACACAGTTCCACCC | CATGATGCTTGATCACATGTCTCG |
| N/A | UniSp6 (QIAGEN) | | |
| Human/Mouse | U6 snRNA (hsa, | | |
| | mmu)(QIAGEN) | | |
| Human/Mouse | hsa-miR-16-5p | | |
| | (QIAGEN) | | |
| Human/Mouse | hsa-miR-21-5p | | |
| | (QIAGEN) | | |
| Human/Mouse | hsa-miR-33a-5p | | |
| | (QIAGEN) | | |
| Human/Mouse | hsa-miR-99a-5p | | |
| | (QIAGEN) | | |
| Human/Mouse | hsa-miR-146b- | | |
| | 5p (QIAGEN) | | |
| Human | hsa-miR-378a- | | |
| | 3p (QIAGEN) | | |
| Mouse | mmu-miR- | | |
| | 378a-3p | | |
| | (QIAGEN) | | |

viii. Glucose Uptake Assay in Cultured 3T3-L1 Adipocytes

3T3-L1 cells were seeded at a density of 2,000 cells/well in a 96-well culture plate and differentiated into mature adipocytes as described above. Adipocytes were then treated with 6×10⁹ particles/mL PBS, THP1-WT-exo, or THP1-IL4-exo for 24 hours. Cells were then starved in serum-free media overnight. The next day, adipocytes were preincubated with KRPH buffer containing 2% bovine serum albumin, 20 mM HEPES, 5 mM KH₂PO₄, 1 mM MgSO₄, 1 mM CaCl₂, 136 mM NaCl, and 4.7 mM KCl, pH 7.4 (all from Sigma Aldrich, USA) for 40 minutes. Cells were then stimulated with 1 μM human insulin (Sigma Aldrich, USA) for 20 minutes. Subsequently, 10 uL/well of 10 mM 2-deoxyglucose (2-DG) was added and incubated for 20 minutes. Next, cells were washed 3× with PBS to remove exogenous 2-DG. Adipocytes were then lysed and 2-DG uptake was processed using a Glucose Uptake Assay Kit (Abcam, USA) according to the manufacturer's protocol. Absorbance reading was measured at OD 412 nm on a microplate reader (Molecular Devices, USA).

ix. Detection of Lipid Vacuoles and Mitochondria Health in Macrophages and 3T3-L1 Adipocytes

For analysis of lipid vacuoles, BMDM or differentiated 3T3-L1 adipocytes were stained with LipidTOX (Invitrogen, USA) (1:250) and Hoechst (1:1000) in Live Cell Imaging Solution (Invitrogen, USA) for 30 minutes in room temperature and imaged using a Zeiss Observer microscope. Alternatively, cells were stained with LipidTOX (Invitrogen, USA) (1:250) for 30 minutes, dissociated with Trypsin-EDTA and analyzed using a CytoFLEX S cytometer (Beckman, USA). For analysis of mitochondrial activity, cells were stained with MitoSOX or tetramethylrhodamine at final concentrations of 5 μM and 0.1 μM, respectively. The cells were then incubated in 37 C for 30 minutes. Cells were then dissociated with Trypsin-EDTA and analyzed using a CytoFLEX S cytometer (Beckman, USA). To measure mitochondrial transition pore opening, BMDM or 3T3-L1 adipocytes were analyzed using the MitoProbe Transition Pore Assay Kit (Invitrogen, USA) according to the manufacturer's protocol. Briefly, cell suspensions were mixed with 2 μM Calcein AM and 160 μM CoCl₂. For negative control, cells were also mixed with 0.2 μM ionomycin. Cells were then analyzed for mitochondrial Calcein AM retention using the CytoFLEX S cytometer (Beckman, USA).

x. Circulating and Tissue Associated Leukocyte Detection Using Flow Cytometry

Mice were anesthetized with isoflurane (Forane, Baxter, USA) and peripheral blood was collected by retro-orbital bleeding with heparinized micro-hematocrit capillary (Fisher Scientific, USA) in tubes containing 0.5M EDTA. Red blood cells were lysed in RBC lysis buffer (BioLegend, USA). Nonspecific binding was blocked with TruStain FcX Ab (BioLegend, USA) for 10 min at 4C in FACS buffer (Ca²⁺/Mg²⁺-free PBS with 2% FBS and 0.5 mM EDTA) before staining with appropriate Abs: CD11b (clone M1/70), Ly-6C (clone HK1.4), CD115 (clone AFS98), and CD45 (clone 30-F11) (all BioLegend, USA) for 30 min at 4 C. The antibody dilutions ranged from 1:200 to 1:100. Splenocytes were isolated using mechanical dissociation. Briefly, spleens were mashed using the bottom of a 3 mL syringe (BD Biosciences, USA). The cells were then passed through a 70 μm cell strainer and incubated in RBC lysis buffer (BioLegend, USA). Nonspecific binding was blocked with TruStain FcX Ab (BioLegend, USA) for 10 min at 4C in FACS buffer before staining with appropriate Abs: CD11b (clone M1/70), Ly-6C (clone HK1.4), Ly-6G (clone 1A8), and CD11c (clone N418). The antibody dilutions ranged from 1:200 to 1:100.

For aorta digestion, single cell suspension from an aorta segment including the aortic arch and thoracic aorta was prepared by incubation with an enzyme mixture containing 400 U/mL Collagenase I, 120 U/mL Collagenase XI, 60 U/mL Hyaluronidase, and 60 U/mL DNase I (all from Sigma Aldrich, USA) in Hank's balanced salt solution for 50 minutes in 37 C. Cells were then passed through a 70-um cell strainer and spun down at 300×g for 5 minutes in 4C. Resulting cell pellet was blocked with TruStain FcX Ab (BioLegend, USA) for 10 min at 4 C in FACS buffer before staining with appropriate Abs: CD11b (clone M1/70), Ly-6C (clone HK1.4), Ly-6G (clone 1A8), CD45 (clone 30-F11), and F4/80 (clone BM8). The antibody dilutions ranged from 1:200 to 1:100.

For adipose tissue and liver digestion, 1 g of epididymal white adipose tissue (eWAT) or 1 g of liver was put in an enzyme mix from the Adipose Tissue or Liver Dissociation Kit (Miltenyi, Germany) and digested using the gentleMACS Dissociator (Miltenyi, Germany) according to the manufacturer's protocol. Cells were then passed through a 100-um cell strainer and centrifuged at 300×g for 10 minutes. Cellular debris was cleared by mixing the cell suspension with Debris Removal Solution (Miltenyi, Germany) and performing density centrifugation according to the manufacturer's protocol. Resulting cell pellet was incubated in RBC Lysis Buffer (BioLegend, USA) and blocked with TruStain FcX Ab (BioLegend, USA) for 10 min at 4C in FACS buffer before staining with appropriate Abs: CD11b (clone M1/70), Ly-6C (clone HK1.4), Ly-6G (clone 1A8), CD45 (clone 30-F11), and F4/80 (clone BM8). The antibody dilutions ranged from 1:200 to 1:100. All euthanized mice were fully perfused with PBS before the tissues were harvested. All flow cytometric analyses were conducted using a CytoFLEX S cytometer (Beckman, USA). Sorting of circulating Ly6C^{hi} monocytes was performed using a FACSAria II Cell Sorter (BD Biosciences, USA). List of antibodies used is available in FIG. 22.

xi. Histological Assessments in Liver and eWAT

Frozen liver tissues were cryosectioned at 10 μm, with 2 sections/mouse/slide. Neutral lipids accumulation was analyzed by staining with oil red O (ORO) (Sigma Aldrich, USA) and counterstaining with modified Mayer's hematoxylin (Thermo Fisher Scientific, USA). ORO-positive areas were captured by a Zeiss Observer microscope and quantified using ImageJ. The average between the two sections is shown for each mouse. Paraffinized liver and eWAT tissues were deparaffinized and cut at 10 μm, with 2 sections/mouse/slide. The slides were then stained with modified Mayer's hematoxylin (Thermo Fisher Scientific, USA) and Eosin Y (Sigma Aldrich, USA) and captured by a Zeiss Observer microscope. Steatotic areas in the liver were measured by ImageJ, with the average between the two sections is shown for each mouse. Average adipocyte sizes were also measured by ImageJ.

For F4/80 or UCP1 staining, deparaffinized eWAT sections underwent heat-induced antigen unmasking in 10 mM citrate buffer with 0.5% Tween 20. Sections were then blocked with 10% normal goat serum and labeled with a primary rabbit anti-mouse F4/80 or UCP1 (Cell Signaling, USA). Endogenous peroxidase activity was then blocked by incubating with 3% H₂O₂ (Sigma Aldrich, USA). Sections were then incubated with SignalStain Boost IHC Detection Reagent anti-rabbit (Cell Signaling, USA) and visualized for peroxidase activity using SignalStain DAB Substrate Kit (Cell Signaling, USA). Sections were counterstained with Vector Methyl Green (Vector Laboratories, USA). Images were captured using a Zeiss Observer microscope. F4/80-positive crown-like structures were enumerated, with the average between the two sections is shown for each mouse.

xii. Measurement of Inflammatory Cytokines and Metabolic Markers in Mice

Peripheral blood was collected as indicated above and spun at 1500×g for 30 minutes in 4C to collect the plasma. Cholesterol and triglycerides levels were measured from plasma using the Cholesterol E Assay Kit or L-Type Triglyceride M Assay Kit (Wako Diagnostics, Fujifilm, Japan). Insulin level was measured in the plasma using a Mouse Insulin ELISA Kit (Abcam, USA). Plasma adiponectin and leptin were measured using a Mouse Adiponectin or Leptin ELISA Kit (Abcam, USA). Plasma cytokines (TNF-α, IFN-γ, IL-6, and IL-1β) were measured using the V-Plex Mouse Custom Cytokine Kit (Meso Scale Discovery, USA). For glucose tolerance test, mice received one dose of glucose (2 g/kg body weight) via i.p. injection after 18 hours of fasting. Blood glucose levels were measured before glucose injection and 30 min, 60 min, 90 min, and 120 min after injection.

xiii. Seahorse Extracellular Flux Analysis

For BMDM, cells were plated at 60,000 cells/well into XFe24 cell culture microplates (Agilent, USA) and incubated overnight at 37C and 5% CO₂. The following day, BMDM were stimulated with PBS, THP1-WT-exo or THP1-IL4-exo for 24 hours. Before analysis, cells were washed with Seahorse XF DMEM assay buffer (Agilent, USA) supplemented with 10 mM glucose (Agilent, USA), 1 mM pyruvate (Agilent, USA), and 2 mM glutamine (Agilent, USA) and incubated for 1 hour at 37C without CO₂. OCR and ECAR were measured using the mitochondrial stress test kit (Agilent, USA) in response to 1 μM Oligomycin, 2 μM Carbonyl cyanide-p-trifluoromethoxyphenylhydrazone (FCCP), and 0.5 μM Rotenone/Antimycin A (R/AA) with the Seahorse XFe-24 Bioanalyzer (Agilent, USA). After OCR measurements, cells were incubated in Hoechst (1:1000) diluted in Live Cell Imaging Solution (Invitrogen, USA) and imaged under a Zeiss Observer microscope. Total cell counts were measured using ImageJ.

For adipocytes, fully differentiated 3T3-L1 cells were plated at 30,000 cells/well into XFe24 cell culture microplates (Agilent, USA) and incubated overnight at 37C and 5% CO₂. The following day, cells were stimulated with PBS, THP1-WT-exo or THP1-IL4-exo for 24 hours. Before analysis, cells were washed with Seahorse XF DMEM assay buffer (Agilent, USA) supplemented with 10 mM glucose (Agilent, USA), 1 mM pyruvate (Agilent, USA) and 2 mM glutamine (Agilent, USA) and incubated for 1 hour at 37C without CO₂. OCR and ECAR were measured using the mitochondrial stress test kit (Agilent, USA) in response to 1 μM Oligomycin, 0.25 μM Carbonyl cyanide-p-trifluoromethoxyphenylhydrazone (FCCP), and 0.5 μM Rotenone/Antimycin A (R/AA) with the Seahorse XFe-24 Bioanalyzer (Agilent, USA). After OCR measurements, cells were incubated in Hoechst (1:1000) diluted in Live Cell Imaging Solution (Invitrogen, USA) and imaged under a Zeiss Observer microscope. Total cell counts were measured using ImageJ.

For ex vivo OCR measurements of eWAT, 10 mg/well of eWAT was washed in Seahorse XF DMEM assay buffer (Agilent, USA) supplemented with 25 mM HEPES (Sigma Aldrich, USA) and placed in a XF24 Islet Capture Microplate (Agilent, USA). The tissue was then locked in a capture screen and incubated in Seahorse XF DMEM assay buffer (Agilent, USA) supplemented with 10 mM glucose (Agilent, USA), 1 mM pyruvate (Agilent, USA) and 2 mM glutamine (Agilent, USA) and incubated for 1 hour at 37C without CO₂. OCR and ECAR were measured using the mitochondrial stress test kit (Agilent, USA) in response to 10 μM Oligomycin, 10 μM Carbonyl cyanide-p-trifluoromethoxyphenylhydrazone (FCCP), and 5 μM Rotenone/Antimycin A (R/AA) with the Seahorse XFe-24 Bioanalyzer (Agilent, USA). Proteins from eWAT were extracted using a Tissue-Tearor in RIPA Buffer (Cell Signaling, USA) containing complete, Mini, EDTA-free Protease Inhibitor Cocktail (Roche, Switzerland) and 1 mM PMSF (Cell Signaling, USA). Protein concentrations were measured using a Pierce BCA Protein Assay Kit (Thermo Fisher Scientific, USA).

All OCR measurements from cells were normalized to cell number and used to calculate various parameters of mitochondrial activity, including basal mitochondrial OCR, proton leak, ATP synthesis, maximal OCR and spare respiratory capacity. All OCR measurements from eWAT were normalized to total proteins and used to calculate similar parameters as indicated above. Data were analyzed using XFe Wave software.

xiv. Statistical Analysis

Statistical analysis was performed with GraphPad Prism v8, using the unpaired, two-tailed, Student's t test (two groups) and one-way or two-way analysis of variance (ANOVA) with post-tests, Holm-Sidak, as indicated in figure legends for multiple groups. *p<0.05, **p<0.01, ***p<0.001 ****p<0.0001. Normality test was performed using the Shapiro-Wilk test on GraphPad Prism v8, with p>0.05 indicating normal distribution. All error bars represent the mean±the standard error of the mean (SEM unless stated). All experiments were repeated at least twice or performed with independent samples.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. 1. B. M. Leon, T. M. Maddox, Diabetes and cardiovascular disease: Epidemiology, biological mechanisms, treatment recommendations and future research. World J Diabetes 6, 1246-1258 (2015).
2. E. P. Kirk, S. Klein, Pathogenesis and pathophysiology of the cardiometabolic syndrome. J Clin Hypertens (Greenwich) 11, 761-765 (2009).
3. M. Y. Donath, D. T. Meier, M. Boni-Schnetzler, Inflammation in the Pathophysiology and Therapy of Cardiometabolic Disease. Endocr Rev 40, 1080-1091 (2019).
4. C. N. Lumeng, S. M. Deyoung, J. L. Bodzin, A. R. Saltiel, Increased inflammatory properties of adipose tissue macrophages recruited during diet-induced obesity. Diabetes 56, 16-23 (2007).
5. C. N. Lumeng, J. B. DelProposto, D. J. Westcott, A. R. Saltiel, Phenotypic switching of adipose tissue macrophages with obesity is generated by spatiotemporal differences in macrophage subtypes. Diabetes 57, 3239-3246 (2008).
6. H. Morinaga, R. Mayoral, J. Heinrichsdorff, O. Osborn, N. Franck, N. Hah, E. Walenta, G. Bandyopadhyay, A. R. Pessentheiner, T. J. Chi, H. Chung, J. G. Bogner-Strauss, R. M. Evans, J. M. Olefsky, D. Y. Oh, Characterization of distinct subpopulations of hepatic macrophages in HFD/obese mice. Diabetes 64, 1120-1130 (2015).
7. D. Y. Oh, H. Morinaga, S. Talukdar, E. J. Bae, J. M. Olefsky, Increased macrophage migration into adipose tissue in obese mice. Diabetes 61, 346-354 (2012).
8. C. K. Glass, J. L. Witztum, Atherosclerosis. the road ahead. Cell 104, 503-516 (2001).
9. M. Back, A. Yurdagul, Jr., I. Tabas, K. Oorni, P. T. Kovanen, Inflammation and its resolution in atherosclerosis: mediators and therapeutic opportunities. Nat Rev Cardiol 16, 389-406 (2019).
10. K. Rahman, E. A. Fisher, Insights From Pre-Clinical and Clinical Studies on the Role of Innate Inflammation in Atherosclerosis Regression. Front Cardiovasc Med 5, 32 (2018).
11. H. Zhao, Q. Shang, Z. Pan, Y. Bai, Z. Li, H. Zhang, Q. Zhang, C. Guo, L. Zhang, Q. Wang, Exosomes From Adipose-Derived Stem Cells Attenuate Adipose Inflammation and Obesity Through Polarizing M2 Macrophages and Beiging in White Adipose Tissue. Diabetes 67, 235-247 (2018).
12. M. Y. Song, S. H. Kim, G. H. Ryoo, M. K. Kim, H. N. Cha, S. Y. Park, H. P. Hwang, H. C. Yu, E. J. Bae, B. H. Park, Adipose sirtuin 6 drives macrophage polarization toward M2 through IL-4 production and maintains systemic insulin sensitivity in mice and humans. Exp Mol Med 51, 1-10 (2019).
13. A. Alisi, G. Carpino, F. L. Oliveira, N. Panera, V. Nobili, E. Gaudio, The Role of Tissue Macrophage-Mediated Inflammation on NAFLD Pathogenesis and Its Clinical Implications. Mediators Inflamm 2017, U.S. Pat. No. 8,162,421 (2017).
14. J. Liu, X. Sun, F. L. Zhang, H. Jin, X. L. Yan, S. Huang, Z. N. Guo, Y. Yang, Clinical Potential of Extracellular Vesicles in Type 2 Diabetes. Front Endocrinol (Lausanne) 11, 596811 (2020).
15. W. Ying, M. Riopel, G. Bandyopadhyay, Y. Dong, A. Birmingham, J. B. Seo, J. M. Ofrecio, J. Wollam, A. Hernandez-Carretero, W. Fu, P. Li, J. M. Olefsky, Adipose Tissue Macrophage-Derived Exosomal miRNAs Can Modulate In Vivo and In Vitro Insulin Sensitivity. Cell 171, 372-384 e312 (2017).
16. W. Ying, H. Gao, F. C. G. Dos Reis, G. Bandyopadhyay, J. M. Ofrecio, Z. Luo, Y. Ji, Z. Jin, C. Ly, J. M. Olefsky, MiR-690, an exosomal-derived miRNA from M2-polarized macrophages, improves insulin sensitivity in obese mice. Cell Metab 33, 781-790 e785 (2021).
17. L. Boucharcychas, P. Duong, S. Covarrubias, E. Alsop, T. A. Phu, A. Chung, M. Gomes, D. Wong, B. Mecchoovet, A. Capili, R. Yamamoto, H. Nakauchi, M. T. McManus, S. Carpenter, K. Van Keuren-Jensen, R. L. Raffai, Macrophage Exosomes Resolve Atherosclerosis by Regulating Hematopoiesis and Inflammation via MicroRNA Cargo. Cell Rep 32, 107881 (2020).
18. L. Boucharcychas, P. Duong, T. A. Phu, E. Alsop, B. Mecchoovet, R. Reiman, M. Ng, R. Yamamoto, H. Nakauchi, W. J. Gasper, K. Van Keuren-Jensen, R. L. Raffai, High glucose macrophage exosomes enhance atherosclerosis by driving cellular proliferation & hematopoiesis. iScience 24, 102847 (2021).
19. P. Duong, A. Chung, L. Bouchareychas, R. L. Raffai, Cushioned-Density Gradient Ultracentrifugation (C-DGUC) improves the isolation efficiency of extracellular vesicles. PLOS One 14, c0215324 (2019).
20. T. A. Sorrentino, P. Duong, L. Bouchareychas, M. Chen, A. Chung, M. S. Schaller, A. Oskowitz, R. L. Raffai, M. S. Conte, Circulating exosomes from patients with peripheral artery disease influence vascular cell migration and contain distinct microRNA cargo. JVS Vasc Sci 1, 28-41 (2020).
21. S. Srinivasan, A. Yeri, P. S. Cheah, A. Chung, K. Danielson, P. De Hoff, J. Filant, C. D. Laurent, L. D. Laurent, R. Magee, C. Moeller, V. L. Murthy, P. Nejad, A. Paul, I. Rigoutsos, R. Rodosthenous, R. V. Shah, B. Simonson, C. To, D. Wong, I. K. Yan, X. Zhang, L. Balaj, X. O. Breakefield, G. Daaboul, R. Gandhi, J. Lapidus, E. Londin, T. Patel, R. L. Raffai, A. K. Sood, R. P. Alexander, S. Das, L. C. Laurent, Small RNA Sequencing across Diverse Biofluids Identifies Optimal Methods for exRNA Isolation. Cell 177, 446-462 c416 (2019).
22. S. Galvan-Pena, L. A. O'Neill, Metabolic reprograming in macrophage polarization. Front Immunol 5, 420 (2014).
23. J. Van den Bossche, J. Baardman, N. A. Otto, S. van der Velden, A. E. Neele, S. M. van den Berg, R. Luque-Martin, H. J. Chen, M. C. Boshuizen, M. Ahmed, M. A. Hoeksema, A. F. de Vos, M. P. de Winther, Mitochondrial Dysfunction Prevents Repolarization of Inflammatory Macrophages. Cell Rep 17, 684-696 (2016).
24. H. Cui, Y. He, S. Chen, D. Zhang, Y. Yu, C. Fan, Macrophage-Derived miRNA-Containing Exosomes Induce Peritendinous Fibrosis after Tendon Injury through the miR-21-5p/Smad7 Pathway. Mol Ther Nucleic Acids 14, 114-130 (2019).
25. M. Ouimet, H. N. Ediriweera, U. M. Gundra, F. J. Sheedy, B. Ramkhelawon, S. B. Hutchison, K. Rinchold, C. van Solingen, M. D. Fullerton, K. Cecchini, K. J. Rayner, G. R. Steinberg, P. D. Zamore, E. A. Fisher, P. Loke, K. J. Moore, MicroRNA-33-dependent regulation of macrophage metabolism directs immune cell polarization in atherosclerosis. J Clin Invest 125, 4334-4348 (2015).
26. M. Ouimet, H. Ediriweera, M. S. Afonso, B. Ramkhelawon, R. Singaravelu, X. Liao, R. C. Bandler, K. Rahman, E. A. Fisher, K. J. Rayner, J. P. Pezacki, I. Tabas, K. J. Moore, microRNA-33 Regulates Macrophage Autophagy in Atherosclerosis. Arterioscler Thromb Vasc Biol 37, 1058-1067 (2017).
27. I. Gerin, L. A. Clerbaux, O. Haumont, N. Lanthier, A. K. Das, C. F. Burant, I. A. Leclercq, O. A. MacDougald, G. T. Bommer, Expression of miR-33 from an SREBP2 intron inhibits cholesterol export and fatty acid oxidation. J Biol Chem 285, 33652-33661 (2010).
28. Z. Wu, Y. Xic, R. F. Morrison, N. L. Bucher, S. R. Farmer, PPARgamma induces the insulin-dependent glucose transporter GLUT4 in the absence of C/EBPalpha during the conversion of 3T3 fibroblasts into adipocytes. J Clin Invest 101, 22-32 (1998).
29. M. S. Afonso, N. Verma, C. van Solingen, Y. Cyr, M. Sharma, L. Peric, E. M. Corr, M. Schlegel, L. C. Shanley, D. Peled, J. Y. Yoo, A. M. Schmidt, E. Mueller, K. J. Moore, MicroRNA-33 Inhibits Adaptive Thermogenesis and Adipose Tissue Beiging. Arterioscler Thromb Vasc Biol 41, 1360-1373 (2021).
30. S. Lhamyani, A.-M. Gentile, R. M. Giráldez-Perez, M. Feijóo-Cuaresma, S. Y. Romero-Zerbo, M. Clemente-Postigo, H. Zayed, W. O. Olivera, F. J. Bermúdez-Silva, J. Salas, C. L. Gómez, A. Hmadcha, N. Hajji, G. O. Fuster, F. J. Tinahones, R. El Bekay, miR-21 mimic blocks obesity in mice: a novel therapeutic option. Molecular Therapy—Nucleic Acids, (2021).
31. Y. Pan, W. Zhao, D. Zhao, C. Wang, N. Yu, T. An, F. Mo, J. Liu, J. Miao, B. Lv, Y. Gu, S. Gao, G. Jiang, Salvianolic Acid B Improves Mitochondrial Function in 3T3-L1 Adipocytes Through a Pathway Involving PPARgamma Coactivator-1alpha (PGC-1alpha). Front Pharmacol 9, 671 (2018).
32. M. Cedikova, M. Kripnerova, J. Dvorakova, P. Pitule, M. Grundmanova, V. Babuska, D. Mullerova, J. Kuncova, Mitochondria in White, Brown, and Beige Adipocytes. Stem Cells Int 2016, U.S. Pat. No. 6,067,349 (2016).
33. C. Y. Woo, J. E. Jang, S. E. Lee, E. H. Koh, K. U. Lee, Mitochondrial Dysfunction in Adipocytes as a Primary Cause of Adipose Tissue Inflammation. Diabetes Metab J 43, 247-256 (2019).
34. S. H. Ro, Y. Jang, J. Bae, I. M. Kim, C. Schaecher, Z. D. Shomo, Autophagy in Adipocyte Browning: Emerging Drug Target for Intervention in Obesity. Front Physiol 10, 22 (2019).
35. D. J. Espiritu, T. Mazzone, Oxidative stress regulates adipocyte apolipoprotein e and suppresses its expression in obesity. Diabetes 57, 2992-2998 (2008).
36. Z. H. Huang, D. Gu, T. Mazzone, Role of adipocyte-derived apoE in modulating adipocyte size, lipid metabolism, and gene expression in vivo. Am J Physiol Endocrinol Metab 296, E1110-1119 (2009).
37. F. Bonacina, D. Coe, G. Wang, M. P. Longhi, A. Baragetti, A. Moregola, K. Garlaschelli, P. Uboldi, F. Pellegatta, L. Grigore, L. Da Dalt, A. Annoni, S. Gregori, Q. Xiao, D. Caruso, N. Mitro, A. L. Catapano, F. M. Marelli-Berg, G. D. Norata, Myeloid apolipoprotein E controls dendritic cell antigen presentation and T cell activation. Nat Commun 9, 3083 (2018).
38. K. Li, D. Ching, F. S. Luk, R. L. Raffai, Apolipoprotein E enhances microRNA-146a in monocytes and macrophages to suppress nuclear factor-kappaB-driven inflammation and atherosclerosis. Circ Res 117, e1-e11 (2015).
39. N. Gaudreault, N. Kumar, J. M. Posada, K. B. Stephens, N. S. Reyes de Mochel, D. Eberle, V. R. Olivas, R. Y. Kim, M. J. Harms, S. Johnson, L. M. Messina, J. H. Rapp, R. L. Raffai, ApoE suppresses atherosclerosis by reducing lipid accumulation in circulating monocytes and the expression of inflammatory molecules on monocytes and vascular endothelium. Arterioscler Thromb Vasc Biol 32, 264-272 (2012).
40. G. Fruhbeck, V. Catalan, A. Rodriguez, B. Ramirez, S. Becerril, J. Salvador, I. Colina, J. Gomez-Ambrosi, Adiponectin-leptin Ratio is a Functional Biomarker of Adipose Tissue Inflammation. Nutrients 11, (2019).
41. M. A. Nguyen, D. Karunakaran, M. Geoffrion, H. S. Cheng, K. Tandoc, L. Perisic Matic, U. Hedin, L. Maegdefessel, J. E. Fish, K. J. Rayner, Extracellular Vesicles Secreted by Atherogenic Macrophages Transfer MicroRNA to Inhibit Cell Migration. Arterioscler Thromb Vasc Biol 38, 49-63 (2018).
42. A. Jaiswal, S. S. Reddy, M. Maurya, P. Maurya, M. K. Barthwal, MicroRNA-99a mimics inhibit M1 macrophage phenotype and adipose tissue inflammation by targeting TNFalpha. Cell Mol Immunol 16, 495-507 (2019).
43. P. Li, C. Fan, Y. Cai, S. Fang, Y. Zeng, Y. Zhang, X. Lin, H. Zhang, Y. Xuc, M. Guan, Transplantation of brown adipose tissue up-regulates miR-99a to ameliorate liver metabolic disorders in diabetic mice by targeting NOX4. Adipocyte 9, 57-67 (2020).
44. D. Ruckerl, S. J. Jenkins, N. N. Laqtom, I. J. Gallagher, T. E. Sutherland, S. Duncan, A. H. Buck, J. E. Allen, Induction of IL-4Ralpha-dependent microRNAs identifies PI3K/Akt signaling as essential for IL-4-driven murine macrophage proliferation in vivo. Blood 120, 2307-2316 (2012).
45. Y. Zhang, C. Li, H. Li, Y. Song, Y. Zhao, L. Zhai, H. Wang, R. Zhong, H. Tang, D. Zhu, miR-378 Activates the Pyruvate-PEP Futile Cycle and Enhances Lipolysis to Ameliorate Obesity in Mice. EBioMedicine 5, 93-104 (2016).
46. D. Pan, C. Mao, B. Quattrochi, R. H. Fricdline, L. J. Zhu, D. Y. Jung, J. K. Kim, B. Lewis, Y. X. Wang, MicroRNA-378 controls classical brown fat expansion to counteract obesity. Nat Commun 5, 4725 (2014).
47. J. Du, P. Zhang, J. Luo, L. Shen, S. Zhang, H. Gu, J. He, L. Wang, X. Zhao, M. Gan, L. Yang, L. Niu, Y. Zhao, Q. Tang, G. Tang, D. Jiang, Y. Jiang, M. Li, A. Jiang, L. Jin, J. Ma, S. Shuai, L. Bai, J. Wang, B. Zeng, Wu, X. Li, L. Zhu, Dietary betaine prevents obesity through gut microbiota-drived microRNA-378a family. Gut Microbes 13, 1-19 (2021).
48. M. C. Runtsch, M. C. Nelson, S. H. Lec, W. Voth, M. Alexander, R. Hu, J. Wallace, C. Petersen, V. Panic, C. J. Villanueva, K. J. Evason, K. M. Bauer, T. Mosbruger, S. Boudina, M. Bronner, J. L. Round, M. J. Drummond, R. M. O'Connell, Anti-inflammatory microRNA-146a protects mice from diet-induced metabolic disease. PLOS Genet 15, e1007970 (2019).
49. V. Usuelli, M. Ben Nasr, F. D'Addio, K. Liu, A. Vergani, B. El Essawy, J. Yang, E. Assi, M. Uchara, C. Rossi, A. Solini, A. Capobianco, E. Rigamonti, L. Potena, M. Venturini, M. Sabatino, L. Bottarelli, E. Ammirati, M. Frigerio, E. Castillo-Leon, A. Maestroni, C. Azzoni, C. Loretelli, A. Joe Seclam, A. K. Tai, I. Pastore, G. Becchi, D. Corradi, G. A. Visner, G. V. Zuccotti, N. B. Chau, R. Abdi, M. G. Pezzolesi, P. Fiorina, miR-21 antagonism reprograms macrophage metabolism and abrogates chronic allograft vasculopathy. Am J Transplant, (2021).
50. F. J. Sheedy, Turning 21: Induction of miR-21 as a Key Switch in the Inflammatory Response. Front Immunol 6, 19 (2015).
51. A. Canfran-Duque, N. Rotllan, X. Zhang, M. Fernandez-Fuertes, C. Ramirez-Hidalgo, E. Araldi, L. Daimiel, R. Busto, C. Fernandez-Hernando, Y. Suarez, Macrophage deficiency of miR-21 promotes apoptosis, plaque necrosis, and vascular inflammation during atherogenesis. EMBO Mol Med 9, 1244-1262 (2017).
52. H. Y. Ling, B. Hu, X. B. Hu, J. Zhong, S. D. Feng, L. Qin, G. Liu, G. B. Wen, D. F. Liao, MiRNA-21 reverses high glucose and high insulin induced insulin resistance in 3T3-L1 adipocytes through targeting phosphatase and tensin homologue. Exp Clin Endocrinol Diabetes 120, 553-559 (2012).
53. T. Han, Y. Lv, S. Wang, T. Hu, H. Hong, Z. Fu, PPARgamma overexpression regulates cholesterol metabolism in human L02 hepatocytes. J Pharmacol Sci 139, 1-8 (2019).
54. A. Klopotek, F. Hirche, K. Eder, PPAR gamma ligand troglitazone lowers cholesterol synthesis in HepG2 and Caco-2 cells via a reduced concentration of nuclear SREBP-2. Exp Biol Med (Maywood) 231, 1365-1372 (2006).
55. T. Yoshikawa, T. Ide, H. Shimano, N. Yahagi, M. Amemiya-Kudo, T. Matsuzaka, S. Yatoh, T. Kitamine, H. Okazaki, Y. Tamura, M. Sckiya, A. Takahashi, A. H. Hasty, R. Sato, H. Sone, J. Osuga, S. Ishibashi, N. Yamada, Cross-talk between peroxisome proliferator-activated receptor (PPAR) alpha and liver X receptor (LXR) in nutritional regulation of fatty acid metabolism. I. PPARs suppress sterol regulatory element binding protein-lc promoter through inhibition of LXR signaling. Mol Endocrinol 17, 1240-1254 (2003).
56. M. Ouimet, S. Koster, E. Sakowski, B. Ramkhelawon, C. van Solingen, S. Oldebeken, D. Karunakaran, C. Portal-Celhay, F. J. Sheedy, T. D. Ray, K. Cecchini, P. D. Zamore, K. J. Rayner, Y. L. Marcel, J. A. Philips, K. J. Moore, Mycobacterium tuberculosis induces the miR-33 locus to reprogram autophagy and host lipid metabolism. Nat Immunol 17, 677-686 (2016).
57. B. Krist, U. Florczyk, K. Pietraszek-Gremplewicz, A. Jozkowicz, J. Dulak, The Role of miR-378a in Metabolism, Angiogenesis, and Muscle Biology. Int J Endocrinol 2015, 281756 (2015).
58. I. Gerin, G. T. Bommer, C. S. McCoin, K. M. Sousa, V. Krishnan, O. A. MacDougald, Roles for miRNA-378/378* in adipocyte gene expression and lipogenesis. Am J Physiol Endocrinol Metab 299, E198-206 (2010).
59. J. L. Escary, H. A. Choy, K. Reue, M. C. Schotz, Hormone-sensitive lipase overexpression increases cholesteryl ester hydrolysis in macrophage foam cells. Arterioscler Thromb Vasc Biol 18, 991-998 (1998).
60. B. Radovic, E. Aflaki, D. Kratky, Adipose triglyceride lipase in immune response, inflammation, and atherosclerosis. Biol Chem 393, 1005-1011 (2012).
61. P. Jha, T. Claudel, A. Baghdasaryan, M. Mueller, E. Halilbasic, S. K. Das, A. Lass, R. Zimmermann, R. Zechner, G. Hoefler, M. Trauner, Role of adipose triglyceride lipase (PNPLA2) in protection from hepatic inflammation in mouse models of steatohepatitis and endotoxemia. Hepatology 59, 858-869 (2014).
62. E. Distel, T. J. Barrett, K. Chung, N. M. Girgis, S. Parathath, C. C. Essau, A. J. Murphy, K. J. Moore, E. A. Fisher, miR33 inhibition overcomes deleterious effects of diabetes mellitus on atherosclerosis plaque regression in mice. Circ Res 115, 759-769 (2014).
63. L. Yvan-Charvet, T. Pagler, E. M. Gautier, S. Avagyan, R. L. Siry, S. Han, C. L. Welch, N. Wang, G. J. Randolph, H. W. Snoeck, A. R. Tall, ATP-binding cassette transporters and HDL suppress hematopoietic stem cell proliferation. Science 328, 1689-1693 (2010).
64. A. R. Tall, P. Costet, N. Wang, Regulation and mechanisms of macrophage cholesterol efflux. J Clin Invest 110, 899-904 (2002).
65. X. Hui, P. Gu, J. Zhang, T. Nie, Y. Pan, D. Wu, T. Feng, C. Zhong, Y. Wang, K. S. Lam, A. Xu, Adiponectin Enhances Cold-Induced Browning of Subcutaneous Adipose Tissue via Promoting M2 Macrophage Proliferation. Cell Metab 22, 279-290 (2015).
66. R. L. Raffai, K. H. Weisgraber, Hypomorphic apolipoprotein E mice: a new model of conditional gene repair to examine apolipoprotein E-mediated metabolism. J Biol Chem 277, 11064-11068 (2002).
67. C. Thery, K. W. Witwer, E. Aikawa, M. J. Alcaraz, J. D. Anderson, R. Andriantsitohaina et al., Minimal information for studies of extracellular vesicles 2018 (MISEV2018): a position statement of the International Society for Extracellular Vesicles and update of the MISEV2014 guidelines. J Extracell Vesicles 7, 1535750 (2018).

1. A method of producing IL-4 exposed M2 macrophage exosomes comprising:
a) culturing macrophage or macrophage precursor cells in the presence of IL-4 in culture media thereby producing IL-4 exposed M2 macrophage exosomes; and
b) isolating the IL-4 exposed M2 macrophage exosomes from the culture media of step a);
wherein the IL-4 exposed M2 macrophage exosomes are enriched with miR-21, miR-99a, miR-146b and miR378a.

2. The method of claim 1, wherein the IL-4 is recombinant human IL-4 cytokine.

3. The method of claim 1, wherein the macrophage or macrophage precursor cells are cultured with IL-4 for at least 24 hours.

4. The method of claim 1, wherein about 6×10⁹ exosomes are secreted per million macrophage or macrophage precursor cells after 24 hours of culturing.

5. The method of claim 1, wherein the macrophage precursor cells are a human monocyte cell line.

6. The method of claim 1, wherein the macrophage or macrophage precursor cells are M2-like macrophage or macrophage precursor cells.

7. The method of claim 6, wherein the M2-like macrophage cells are from a THP-1 cell line.

8. The method of claim 1, wherein the isolating step comprises cushioned-density gradient ultracentrifugation.

9. The method of claim 1, wherein the isolated IL-4 exposed M2 macrophage exosomes are between 80-110 nm.

10. A method of reprogramming macrophages comprising exposing macrophages to IL-4 exposed M2 macrophage exosomes, wherein immune and/or metabolic properties are altered in the macrophages.

11.-74. (canceled)

75. A recombinant macrophage-derived exosome enriched with microRNA including miR-21, miR-99a, miR-146b and miR378a.

76.-81. (canceled)

82. The method of claim 1, further comprising differentiating macrophage precursor cells into macrophages prior to step a).

83. The method of claim 1, further comprising exposing the macrophage or macrophage precursor cells with phorbol 12-myristate 13-acetate (PMA) prior to step a).

84. The method of claim 1, wherein the macrophage or macrophage precursor cells are primary cells.

85. The method of claim 1, wherein the macrophage or macrophage precursor cells are bone marrow derived macrophages (BMDMs).

86. The recombinant macrophage-derived exosome of claim 75, wherein the recombinant macrophage-derived exosome is derived from bone marrow derived macrophages (BMDMs) or THP derived macrophages.

87. The recombinant macrophage-derived exosome of claim 75, wherein the miR-21, miR-99a, miR-146b and miR378a are enriched compared to a macrophage-derived exosome not exposed to IL-4.
